(19) United States
(12) Patent Application Publication
(10) Pub. No.: US 2021/0378263 A1
(43) Pub. Date: Dec. 9, 2021

(54) BIOREMEDIATION OF XENOBIOTICS IN THE HONEY BEE HIVE

(71) Applicant: UNIVERSITY OF FLORIDA RESEARCH FOUNDATION, Gainesville, FL (US)

(72) Inventors: Massimo MADDALONI, Gainesville, FL (US); David Wayne PASCUAL, Gainesville, FL (US); James ELLIS, High Springs, FL (US)

(21) Appl. No.: 17/287,717

(22) PCT Filed: Oct. 25, 2019
(86) PCT No.: PCT/US2019/058146

§ 371 Date: Apr. 22, 2021

Related U.S. Application Data
(60) Provisional application No. 62/750,396, filed on Oct. 25, 2018.

(51) Int. Cl. A23K 20/189; A61K 35/747; C12N 15/52; A23K 50/90

(52) U.S. Cl. CPC A23K 20/189; A61K 35/747; C12N 15/52; A23K 50/90; C12Y 401/01002; C12Y 301/01008; C12Y 301/08001; C12Y 308/01008; C12Y 301/01001

(57) ABSTRACT

Described herein are engineered cells, enzymes, methods of use, and bee bread incorporating engineered cells and enzymes as described herein. In certain aspects, described herein are a bacterium containing therein one or more stably-expressing expression vectors for exogenous expression of one or more recombinant carboxylesterase enzymes or oxalate decarboxylase enzymes, thereby providing the engineered cell an exogenous pathway for hydrolyzing ester bonds or removing a carboxyl group. Engineered cells and recombinant enzymes as described herein can be incorporated into bee bread to be fed to a member of the Apidae family of bees or of the Apis or Bombus genus. In additional aspects, such bacteria can also be selected and amplified from the milieu of the hive microorganisms and in some cases they can be molecularly bred to enhance their metabolic capabilities without genetic engineering.

### CROSS-REFERENCE TO RELATED APPLICATION

This application claims priority to U.S. Provisional application entitled “BIOREMEDIATION OF XENOBIOTICS IN THE HONEY BEE HIVE,” having Ser. No. 62/750,396, filed on Oct. 25, 2018, which is entirely incorporated herein by reference.

### SEQUENCE LISTING

This application contains a sequence listing filed in electronic form as an ASCII.txt file entitled “222107_2230_seq_listing_ST25.txt”, created on Oct. 25, 2019. The content of the sequence listing is incorporated herein in its entirety.

### BACKGROUND

Losses in honey bee (Apis mellifera) colonies are estimated to be ˜40% annually, warranting robust research to curb the economic and ecological damage. According to the 2017 USDA Annual Report, honey bees “ . . . add at least $15 billion to the value of U.S. agriculture annually through increased yields and superior-quality harvests.”

Losses are believed to be in part attributed to colony collapse disorder (CCD), an umbrella name defining a syndrome more than a specific disease, as well as to other “disappearing diseases”. If left unattended, this can have significant impact in sustaining current agricultural practices as well as having negative environmental consequences.

There is debate regarding the responsible etiological agent(s), be these chemical or biological in nature. There is a substantial agreement, however, that the causes of the losses are attributable to three large categories of stressors: 1) the spread of pathogens and pests fostered by anthropic commercial activities; 2) the lack of a constant supply of food source throughout the year; and 3) the widespread use of pesticides both in domestic and agricultural settings. Strategies to mitigate and/or reverse losses in honey bees and other pollinators are currently lacking.

Accordingly, there is a need to address the aforementioned deficiencies and inadequacies.

### SUMMARY

Described herein are engineered cells. In certain aspects, engineered cells can comprise a bacterium containing therein one or more stably-expressing expression vectors for exogenous expression of one or more recombinant carboxylesterase enzymes, thereby providing the engineered cell an exogenous pathway for hydrolyzing ester bonds. The bacterium can be a bacteria of the genus Escherichia, Lactobacillus, Fructobacillus, Gilliamella, Snodgrassella, Actinobacteria, Parasaccharibacter. Pseudomonas, Stenotrophomonas, Sphigobacterium, or Bacillus.

In certain embodiments, one or more stably-expressing vectors according to the present disclosure can comprise one or more coding sequences for a recombinant carboxylesterase or one or more coding sequences for a neonicotinoid and/or neonicotinoid metabolite degradation recombinant enzyme.

In certain embodiments, the recombinant carboxylesterase enzymes can be Methyl Parathion Hydrolase (MPH), Pyrethroid Hydrolase (PytH), E3 carboxylesterase, E3^{Trp251Leu} carboxylesterase, Deinococcus radiodurans phosphotriesterase, or Sulpholobus acidocaldarius phosphotriesterase. In certain aspects, recombinant carboxylesterase enzymes as described herein, such as Deinococcus radiodurans phosphotriesterase, or Sulpholobus acidocaldarius phosphotriesterase, are thermostable recombinant carboxylesterase enzymes. In certain embodiments, the recombinant carboxylesterase enzymes can comprise or be encoded by sequences comprising one or more of sequences 90% to 100% identical to SEQ ID NOs: 1-5.

Described herein is modified bee bread. In embodiments according to the present disclosure, modified bee bread as described herein can comprise isolated wild-type or synthetic bee bread, one or more purified recombinant carboxylesterases, one or more purified neonicotinoid and/or neonicotinoid metabolite degradation recombinant enzymes, one or more engineered cells, or any combination thereof. The one or more purified recombinant carboxylesterases, one or more purified neonicotinoid and/or neonicotinoid metabolite degradation recombinant enzymes, or both can be present in an amount effective to reduce the amount of active ingredient of one or more insecticides from a first level to a second level, wherein the second level is less than the first. The one or more engineered cells can be present in an amount effective to reduce the amount of active ingredient of one or more insecticides from a first level to a second level, wherein the second level is less than the first. The one or more insecticides can be one or more insecticides from the pyrethroid, synthetic pyrethroid, organophosphate, or neonicotinoid class of insecticides.

Described herein are methods of providing protection from insecticides and/or methods of providing a subject in need thereof protection from insecticides. Methods as described herein can comprise, in certain aspects, providing a bee bread (wild-type or synthetic); providing one or more purified recombinant carboxylesterases, one or more purified neonicotinoid and/or neonicotinoid metabolite degradation recombinant enzymes, one or more engineered cells, or any combination thereof; and introducing one or more purified recombinant carboxylesterases, one or more purified neonicotinoid and/or neonicotinoid metabolite degradation recombinant enzymes, one or more engineered cells, or any combination thereof into the bee bread. The one or more purified recombinant carboxylesterases, one or more purified neonicotinoid and/or neonicotinoid metabolite degradation recombinant enzymes, or both can be present in an amount effective to reduce the amount of active ingredient of one or more insecticides from a first level to a second level, wherein the second level is less than the first. The one or more engineered cells can be present in an amount effective to reduce the amount of active ingredient of one or more insecticides from a first level to a second level, wherein the second level is less than the first. The one or more insecticides can be one or more insecticides from the pyrethroid, synthetic pyrethroid, organophosphate, or neonicotinoid class of insecticides.

Also described herein are kits, comprising: one or more bacterial cells; and one or more expression vectors comprising one or more recombinant carboxylesterase coding sequences and/or one or more coding sequences for a neonicotinoid and/or neonicotinoid metabolite degradation recombinant enzyme.

In certain aspects, described herein is a method of creating an engineered cell, comprising: providing wild-type bee bread; isolating one or more bacteria from the wild-type bee bread; introducing one or more expression vectors comprising one or more recombinant carboxylesterase coding sequences and/or one or more coding sequences for a neonicotinoid and/or neonicotinoid metabolite degradation recombinant enzyme into the one or more bacteria; selecting one or more stably-expressing recombinant carboxylesterase bacteria after the one or more expression vectors are introduced; and isolating the stably-expressing recombinant carboxylesterase bacteria.

Described herein are methods of treating a subject for pesticide exposure, the methods comprising delivering a modified bee bread as described herein to a subject in need thereof. The modified bee bread can be delivered to the subject in an amount effective to reduce the active amount of one or more insecticides from a first level to a second level, wherein the second level is less than the first. The one or more insecticides are one or more insecticides from the pyrethroid, synthetic pyrethroid, organophosphate, or neonicotinoid class of insecticides.

In further aspects, described herein are engineered cells, comprising a bacterium containing therein one or more stably-expressing expression vectors for exogenous expression of one or more recombinant oxalate decarboxylase enzymes, thereby providing the engineered cell an exogenous pathway for removing carboxyl groups. The bacterium can be a bacteria of the genus Escherichia, Lactobacillus, Fructobacillus, Gilliamella, Snodgrassella, Actinobacteria, Parasaccharibacter. Pseudomonas, Stenotrophomonas, Sphigobacterium, or Bacillus. The one or more stably-expressing vectors can comprise one or more coding sequences for a recombinant oxalate decarboxylase. The oxalate decarboxylase can be a B. subtilis, Burkholderia pseudomallei, or Pantoea allii oxalate decarboxylase. In an embodiment, the oxalate decarboxylase is a B. subtilis oxalate decarboxylase. In an embodiment, the oxalate decarboxylase is a Burkholderia pseudomallei oxalate decarboxylase. In an embodiment, the oxalate decarboxylase is a Pantoea allii oxalate decarboxylase.

The one or more stably-expressing vectors can comprise one or more coding sequences with about 90% to about 100% sequence identify with SEQ ID NOs: 6-11. The one or more stably-expressing vectors can comprise one or more coding sequences with about 90% to about 100% sequence identify with SEQ ID NO: 6. The one or more stably-expressing vectors can comprise one or more coding sequences with about 90% to about 100% sequence identify with SEQ ID NO: 7. The one or more stably-expressing vectors can comprise one or more coding sequences with about 90% to about 100% sequence identify with SEQ ID NO: 8. The one or more stably-expressing vectors can comprise one or more coding sequences with about 90% to about 100% sequence identify with SEQ ID NO: 9. The one or more stably-expressing vectors can comprise one or more coding sequences with about 90% to about 100% sequence identify with SEQ ID NO: 10. The one or more stably-expressing vectors can comprise one or more coding sequences with about 90% to about 100% sequence identify with SEQ ID NO: 11.

In embodiments, described herein are modified bee breads, comprising bee bread; and one or more purified recombinant oxalate decarboxylases, one or more engineered cells, or both. In certain aspects, the bee bread can be wild-type bee bread. In certain aspects, the bee bread can be synthetic bee bread. The one or more purified recombinant oxalate decarboxlyases are B. subtilis, Burkholderia pseudomallei, or Pantoea allii oxalate decarboxylases. In an embodiment, the oxalate decarboxylase is a B. subtilis oxalate decarboxylase. In an embodiment, the oxalate decarboxylase is a Burkholderia pseudomallei oxalate decarboxylase. In an embodiment, the oxalate decarboxylase is a Pantoea allii oxalate decarboxylase.

In certain aspects, one or more purified recombinant oxalate decarboxylases are present in an amount effective to reduce the amount of active ingredient of one or more insecticides from a first level to a second level, wherein the second level is less than the first.

In certain aspects, the one or more engineered cells are present in an amount effective to reduce the amount of active ingredient of oxalic acid from a first level to a second level, wherein the second level is less than the first.

Described herein are additional embodiments of methods of providing protection from insecticides, comprising providing a bee bread; providing one or more purified recombinant oxalate decarboxylases, one or more engineered cells, or both; and introducing the one or more purified recombinant oxalate decarboxylases, one or more engineered cells, or both into the bee bread. Such a bee bread can be provided to a subject in need thereof, a plurality of subjects in need thereof, or a hive or a subject in need thereof. In certain aspects, the bee bread can be wild-type bee bread. In certain aspects, the bee bread can be synthetic bee bread. One or more purified recombinant oxalate decarboxlyases according to methods as described herein can be B. subtilis, Burkholderia pseudomallei, or Pantoea allii oxalate decarboxylases. In an embodiment, the oxalate decarboxylase is a B. subtilis oxalate decarboxylase. In an embodiment, the oxalate decarboxylase is a Burkholderia pseudomallei oxalate decarboxylase. In an embodiment, the oxalate decarboxylase is a Pantoea allii oxalate decarboxylase. The one or more oxalate carboxylesterases are present in an amount effective to reduce the amount of active ingredient of of one or more insecticides from a first level to a second level, wherein the second level is less than the first level. The one or more engineered cells are present in an amount effective to reduce the amount of active ingredient of oxalic acid from a first level to a second level, wherein the second level is less than the first level.

Described herein are additional embodiments of kits, comprising one or more bacterial cells and one or more expression vectors comprising one or more recombinant oxalate decarboxylase coding sequences. The one or more coding sequences have about 90% to about 100% sequence identify with SEQ ID NOs 6-11. The one or more stably-expressing vectors can comprise one or more coding sequences with about 90% to about 100% sequence identify with SEQ ID NO. 6. The one or more stably-expressing vectors can comprise one or more coding sequences with about 90% to about 100% sequence identify with SEQ ID NO. 7. The one or more stably-expressing vectors can comprise one or more coding sequences with about 90% to about 100% sequence identify with SEQ ID NO. 8. The one or more stably-expressing vectors can comprise one or more coding sequences with about 90% to about 100% sequence identify with SEQ ID NO 9. The one or more stably-expressing vectors can comprise one or more coding sequences with about 90% to about 100% sequence identify with SEQ ID NO. 10. The one or more stably-expressing vectors can comprise one or more coding sequences with about 90% to about 100% sequence identify with SEQ ID NO. 11.

Described herein are additional embodiments of methods of creating an engineered cell, comprising providing wild-type bee bread, isolating one or more bacteria from the wild-type bee bread, introducing one or more expression vectors comprising one or more recombinant oxalate decarboxylase coding sequences into the one or more bacteria, selecting one or more stably-expressing recombinant oxalate decarboxylase bacteria after the one or more expression vectors are introduced; and isolating the stably-expressing recombinant oxalate decarboxylase bacteria. Described herein are also cells created by this method.

Described herein are additional embodiments of methods of treating a subject for pesticide exposure, the method comprising delivering a modified bee bread as described herein to a subject in need thereof. The modified bee bread can be delivered to the subject in an amount effective to reduce the active amount of of one or more insecticides from a first level to a second level, wherein the second level is less than the first.

In further aspects, described herein are additional embodiments of engineered cells. In an embodiment, an engineered cells comprises a bacterium containing therein one or more stably-expressing expression vectors for exogenous expression of one or more recombinant 6-chloronicotinic acid degradation enzymes, thereby providing the engineered cell an exogenous pathway for degrading 6-chloronicotinic acid. In certain aspects, the bacterium can be a bacteria of the genus Escherichia, Lactobacillus, Fructobacillus, Gilliamella, Snodgrassella, Actinobacteria, Parasaccharibacter, Pseudomonas, Stenotrophomonas, Sphigobacterium, or Bacillus. The one or more stably-expressing vectors can comprise one or more coding sequences for a recombinant 6-chloronicotinic acid chlorohydrolase (cch2) or a cassette comprising a a recombinant 6-chloronicotinic acid chlorohydrolase (cch2) fused to a transporter. The recombinant 6-chloronicotinic acid chlorohydrolase can be a Bradyrhizobiaceae bacterium cch2. The one or more stably-expressing vectors can comprise one or more coding sequences encoding cch2 with about 90% to about 100% sequence identify with the cch2 sequence of SEQ ID NOs: 12-13.

In additional aspects, described herein are a modified bee bread, comprising bee bread; and one or more purified recombinant 6-chloronicotinic acid chlorohydrolases (cch2), one or more engineered cells, or both. The bee bread can be a wild-type bee bread. The bee bread can be a synthetic bee bread. The one or more purified recombinant 6-chloronicotinic acid chlorohydrolases can be a Bradyrhizobiaceae bacterium 6-chloronicotinic acid chlorohydrolase. The one or more purified recombinant 6-chloronicotinic acid chlorohydrolases can fused to a transporter on the n- or c-terminus.

The one or more purified recombinant 6-chloronicotinic acid chlorohydrolases can be present in an amount effective to reduce the amount of active ingredient of one or more insecticides from a first level to a second level, wherein the second level is less than the first.

The one or more engineered cells can be present in an amount effective to reduce the amount of 6-CNA from a first level to a second level, wherein the second level is less than the first.

Described herein are embodiments of methods of providing protection from insecticides, comprising providing a bee bread, providing one or more purified recombinant 6-chloronicotinic acid chlorohydrolases, one or more engineered cells, or both, and introducing the one or more purified recombinant 6-chloronicotinic acid chlorohydrolases, one or more engineered cells, or both into the bee bread. The bee bread can be a wild-type bee bread. The bee bread can be a synthetic bee bread. The one or more purified recombinant 6-chloronicotinic acid chlorohydrolases can be a Bradyrhizobiaceae bacterium 6-chloronicotinic acid chlorohydrolase. The one or more 6-chloronicotinic acid chlorohydrolases can be present in an amount effective to reduce the amount of active ingredient of of one or more insecticides from a first level to a second level, wherein the second level is less than the first level. The one or more engineered cells can be present in an amount effective to reduce the amount of 6-CNA from a first level to a second level, wherein the second level is less than the first level.

Described herein are further embodiments of kits, comprising one or more bacterial cells, and one or more expression vectors comprising one or more recombinant 6-chloronicotinic acid chlorohydrolase coding sequences. The one or more coding sequences can have about 90% to about 100% sequence identify with the 6-chloronicotinic acid chlorohydrolase sequence of SEQ ID NOs: 12-13. A coding sequence for a transporter can also be contained therein.

Further described herein are methods of creating an engineered cell, comprising providing wild-type bee bread, isolating one or more bacteria from the wild-type bee bread, introducing one or more expression vectors comprising one or more recombinant 6-chloronicotinic acid chlorohydrolase coding sequences into the one or more bacteria, selecting one or more stably-expressing recombinant 6-chloronicotinic acid chlorohydrolase bacteria after the one or more expression vectors are introduced, and isolating the stably-expressing recombinant 6-chloronicotinic acid chlorohydrolase bacteria. Described herein are engineered cells created by methods as described herein.

Described herein are additional embodiments of methods of treating a subject for pesticide exposure, the method comprising delivering a modified bee bread as described herein to a subject in need thereof, or a hive of a subject in need thereof.

According to embodiments of the present disclosure, the modified bee bread can be delivered to the subject in an amount effective to reduce the active amount of of one or more insecticides from a first level to a second level, wherein the second level is less than the first. The cell can further comprise a 6-HNA degradation operon. A cell with a functional 6-HNA degradation operon such as those described herein can also be added to bee bread as described herein. In an embodiment, the modified bee bread can further comprise one or more engineered cells expressing a 6-HNA degradation operon. In embodiments, the one or more engineered cells express a 6-HNA degradation operon or one or more genes of such according to the examples below. The one or more cells can comprise a 6-HNA degradation operon. Embodiments of methods can further comprise introducing one or more expression vectors encoding one or more recombinant gene products of a 6-HNA degradation operon. The one or more expression vectors encoding one or more recombinant gene products of a 6-HNA degradation operon comprise one or more recombinant nucleotide sequences encoding one or more or SEQ ID. NOs: 14-24.

### BRIEF DESCRIPTION OF THE DRAWINGS

Many aspects of the disclosed devices and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIG. 1 is an overview of aspects of the present disclosure.

FIGS. 2A-2D are photographs depicting expression of carboxylesterase in E. coli (FIG. 2A) and Gilliamella apicola (FIG. 2C). (FIG. 2B) and (FIG. 2D) show the empty vector control. Chromogenic substrate is X-caprylate (5-Bromo-4-chloro-3-indoxyl octanoate) overlaid onto colonies.

FIGS. 3A-3B are photographs demonstrating expression of carboxylesterase in Lactobacillus kunkeei (FIG. 3B) and Fructobacillus fructosus (FIG. 3A). The chromogenic substrate is 4-nitrophenyl acetate added to the bacterial cell suspension.

FIGS. 4A-4B represent flow cytometry data of S. alvi chromosomally transformed with two integrative vectors expressing the m-Cherry fluorescent protein.

FIGS. 5A-5B are confocal micrographs of guts of honey bees fed recombinant LAB bearing pGnMM70 expressing m-Cherry. This approach will enable us to define the best bacteria-plasmid combinations. This construct is silent in L. kunkeei cells (FIG. 5A) which regain fluorescence when extracted from the rectum and plated on MRS+erythromycin (FIG. 5B). Bacteria are shown in red (m-Cherry expression), gut cell nuclei are shown in blue (4%6-diamidino-2-phenylindole, DAPI) (see representative annotations).

FIG. 6 is a graph depicting 3-log growth of m-Cherry transformed F. fructosus and L. kunkeei in high-pollen simulated bee bread (50% commercial pollen, 20% fructose). Transformed bacterial colonies remained Eryth^{Res} m-Cherry⁺ even if no antibiotic was present in the bee bread. pH decreased from 4.5 to 3.5. At the end no colonies detected even without erythromycin. This example provides a proof that recombinant strains as described herein maintain metabolic capabilities in a milieu that is very similar to their target environment.

FIGS. 7A-7B are graphs depicting data from a 40xLC₅₀ cypermethrin challenge. Simulated bee bread was spiked with the highly toxic cypermethrin, fermented with a mix of selected bacteria and fed to the bees. Cypermethrin concentration was adjusted to deliver 40xLC₅₀ daily (assuming that each bee would take 25 ml of the simulated bee bread daily). Cypermethrin-spiked medium was not withdrawn at any time. FIG. 7A depicts a survival curve for the bacteria (percent survival), and FIG. 7B illustrates time to death (days). A 40xLC₅₀ challenge represents a concentration that is 5-9 log higher than that honey bees are likely to meet in the field.

FIG. 8A is Coomassie staining showing the purified recombinant naturally-occurring variant (Trp251Leu) of the E3 carboxylesterase from Lucilia cuprina, In this experiment the recombinant carboxylase was expressed as a fusion to Maltose Binding Protein (Construct pMALcarboxylaseFusions top map).

FIGS. 8B-8C illustrate data from a 40xLC₅₀ cypermethrin challenge. 20% fructose syrup was spiked with cypermethrin and bioremediated with the purified carboxylesterase. Cypermethrin concentration was adjusted to deliver 40xLD₅₀ daily (assuming that each bee would take 25 μl of the syrup daily). Each treatment consisted of 5 batches, each containing 10 bees. Cypermethrin-spiked syrup was not withdrawn at any time. Media consumption shows that bees kept feeding during the experiment. FIG. 8B illustrates a survival curve of bees without protein (−) (bottom; circles), with protein (+) (middle; squares), and control (top; triangles). FIG. 8C shows the amount of medium consumed without protein (−) and with protein (+).

FIG. 9 is a table that is representative of selective reaction monitoring/multiple reaction monitoring (SRM/MRM) transitions which can be used for quantitation and qualification according to compositions and methods of the present disclosure.

FIG. 10 is a survival curve of a 40xLD50 cypermethrin challenge delivered by fructose syrup as in the experiment of FIGS. 8B-8C. Black line (very top line with circles). Fructose syrup only. Red line (bottom line with squares). Fructose syrup+cypermethrin Green line (middle line with triangles). Fructose syrup+cypermethrin+protein. Blue line (second to the top line with upside down triangles). Fructose syrup made with AB-mineral medium supernatant of the consortium, spiked with cypermethrin at the beginning of the growth.

FIG. 11 is a diagram illustrating an embodiment of a molecular breeding procedure according to the present disclosure. Over the course of one year, bacteria, which can be derived from ripening bee bread, can be cycled through bentonic growth on rich media or AB mineral media and planctonic growth on AB mineral media. AB mineral media can contain the 4 insecticides (the neonicotinoid, imidacloprid; the neonicotinoid, clothianidin; the synthetic pyrethroid, cypermethrin; and the oraganophosphate, methylparathion) as the sole carbon source. After four cycles, bacteria can be transferred in planctonic AB minimal media and subcultured for about six months. At the end of this period, the surviving bacteria can be plated on rich media, selected for morphological differences and further characterized.

FIG. 12 The 50 pure culture bacteria described in the following FIG. 13 were spotted on a BHI (Brain heart infusion) Petri dish and overlaid with X-caprylate, a chromogenic substrate which detects carboxylesterase activities by turning blue. The activity was arbitrarily assigned to 5 groups: +++ robust blue color within 5 minutes; ++ robust blue color, + weak blue color within 1 hour; +/− very pale color, marginally detectable at one hour, and − no blue color detectable.

FIG. 13 is a table of clones of FIG. 12 showing results of 16S DNA genotyping of 50 single-colony clones isolated from a molecular breeding experiment. Using the specific AB-insecticides medium, colonies were isolated and marked below in color. Genomic DNA was isolated from each colony, and amplified with universal primers. The resulting DNA bands were Sanger-sequenced, and sequences were matched against existing data banks. The table shows the best match of the clones isolated from bee bread; however in most cases, the match was not 100%, suggesting that these isolates may represent novel subtypes.

FIG. 14 illustrates aspects of neonicotinoid breakdown, in particular into 6-chloronicotinic acid (6-CNA). 6-CNA can be recalcitrant to biodegradation due to the chlorine atom present. That said, 6-CNA can be funneled into a functional degradation pathway according to additional aspects of the present disclosure.

FIGS. 15A and 15B illustrate aspects of operons for nicotinic acid degradation according to the present disclosure.

FIG. 16 illustrates embodiments of expression vectors for neonicotinoid or nicotinic acid degradation according to the present disclosure.

FIG. 17 illustrates embodiments of expression vectors for neonicotinoid or nicotinic acid degradation according to the present disclosure. Regarding certain vectors of the present disclosure, pMB1 refers to cassettes for E. coli constructs, whereas the RK2 refers to cassettes already moved into a backbone with larger applications, including Pseudomonas.

FIG. 18 illustrates embodiments of carboxylesterase expression vectors according to the present disclosure.

FIGS. 19A-19B. Analysis of Fructobacillus genome. ApaI (GGGCCC) and BamHI (GGATCC) sites, used for cloning, are marked in bold. (FIG. 19A) Region surrounding the putative promoter element driving the expression on the fructokinase-glucose-6-phospate isomerase di-cistronic operon (bp 15637-15752). The lower line schematically depicts how this region was incorporated into the pGnMM37 expression vector. Shine-Dalgarno sequence is indicated in small letters and underlined. Putative TATA box is underlined. (FIG. 19B) Region surrounding a putative promoter element driving the expression of a 14 ribosomal protein gene cluster (bp 18008-18309). Shine-Dalgarno sequence is indicated in small letters and underlined. The lower line schematically depicts how this region was incorporated into the pGnMM38 expression vector.

FIG. 20 is a schematic representation of pGnMM56 and pGnMM64, constitutively expressing Bacillus subtilis (B. subtilis) oxalate decarboxylase under the control of the universal p23 and F. fructosus 18008-18309 promoter, respectively. Both constructs use a pIB184 backbone enhanced with a transcription terminator.

FIGS. 21A-21D are photographs showing oxalate degradation in Petri dishes. Fructobacillus fructosus was plated on MRS, fructose, Mn⁺⁺ plates, overlaid with a top agar containing calcium oxalate, as described in Materials and methods. Clockwise, starting top left: untransformed bacteria control (FIG. 21A), naked vector (FIG. 21B), pGnMM56 (FIG. 21C), and pGnmm64 (FIG. 21D). Halos of calcium oxalate depletion are evident only in recombinant strains expressing the Bacillus subtilis oxalate decarboxylase (bottom panels).

FIGS. 22A-22D are photographs showing oxalate degradation in Petri dishes. Lactobacillus kunkeei was plated on MRS, fructose, Mn⁺⁺ plates, overlaid with a top agar containing calcium oxalate, as described in Materials and methods. Clockwise, starting top left: untransformed bacteria control (FIG. 22A), naked vector (FIG. 22B), pGnMM56 (FIG. 22C), and pGnmm64 (FIG. 22D). Halos of calcium oxalate depletion are evident only in recombinant strains expressing the Bacillus subtilis oxalate decarboxylase (bottom panels).

FIG. 23 is a graph of oxalate titration in liquid cultures. Different strains were grown on MRS, fructose, Mn⁺⁺ and potassium oxalate was added as explained in Material & Methods. Supernatants were titrated with an oxalate assay kit from Sigma-Aldrich. Different concentrations of potassium oxalate were tested. Each bar represents two independent titrations. A small error was consistently detected across different experiments and warranted only two repetitions. For each treatment the amount of potassium oxalate in the supernatant of the non-recombinant controls was set to 100%. Bioremediation of potassium oxalate in the supernatants consistently reaches 95-100% across concentration between 1 to 20 mmoles l⁻¹ of potassium oxalate. 20 mmoles l⁻¹ is approximately 13 times the linear detection limit of the oxalate assay kit. No bioremediation can be observed at 100 mmoles l⁻¹ of potassium oxalate. However, at this concentration bacterial growth is severely stunted.

FIG. 24 Pseudomonas putida KT2440 engineered with a construct (pGnMM105) bearing the cassette borne on pGnMM83 (FIG. 17), which confers the capability to use 6-chloronicotinic acid as a sole carbon source, provided that a nicotinic acid degradation pathway (FIG. 14) is functional. Four clones are shown.

FIG. 25 shows expression of Pseudomonas Wbc-3 carboxylesterase.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of melittology, apiology, microbiology, molecular biology, toxicology, organic chemistry, analytical chemistry, and biochemistry.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### Definitions

Unless otherwise defined, all technical and scientific terms used herein have the same meaning as commonly understood by one of ordinary skill in the art of molecular biology, medicinal chemistry, and/or organic chemistry. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, a “subject” can be a member of the Apidae family of bees. In certain embodiments, a subject is a bee of the Apis or Bombus genus. A “subject in need thereof” can be a subject as described herein (a bee of the Apis or Bombus genus) that is subjected to, or at risk for, pesticide exposure. In certain embodiments, a “subject in need thereof” is a honey bee (Apis mellifera) that is subjected to, or at risk for, pesticide exposure.

As used herein, “pesticide” or “insecticide” can refer to a compound of the organophostphate, pyrethroid, or neonicotinoid class of insecticides.

As used herein, “control” is an alternative subject or sample used in an experiment for comparison purposes and included to minimize or distinguish the effect of variables other than an independent variable.

As used herein, “overexpressed” or “overexpression” refers to an increased expression level of an RNA or protein product encoded by a gene as compared to the level of expression of the RNA or protein product in a normal or control cell.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins.

As used herein, “nucleic acid” and “polynucleotide” generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide includes DNAs or RNAs as described above that contain one or more modified bases. Thus, DNAs or RNAs comprising unusual bases, such as inosine, or modified bases, such as tritylated bases, to name just two examples, are polynucleotides as the term is used herein. “Polynucleotide” and “nucleic acids” also includes PNAs (peptide nucleic acids), phosphorothioates, and other variants of the phosphate backbone of native nucleic acids. Natural nucleic acids have a phosphate backbone, artificial nucleic acids may contain other types of backbones, but contain the same bases. Thus, DNAs or RNAs with backbones modified for stability or for other reasons are “nucleic acids” or “polynucleotide” as that term is intended herein.

As used herein, “deoxyribonucleic acid (DNA)” and “ribonucleic acid (RNA)” generally refer to any polyribonucleotide or polydeoxribonucleotide, which may be unmodified RNA or DNA or modified RNA or DNA. RNA may be in the form of a tRNA (transfer RNA), snRNA (small nuclear RNA), rRNA (ribosomal RNA), mRNA (messenger RNA), anti-sense RNA, RNAi (RNA interference construct), siRNA (short interfering RNA), or ribozymes.

As used herein, “nucleic acid sequence” and “oligonucleotide” also encompasses a nucleic acid and polynucleotide as defined above.

As used herein, “DNA molecule” includes nucleic acids/polynucleotides that are made of DNA.

As used herein, “wild-type” is the typical form of an organism, variety, strain, gene, protein, or characteristic as it occurs in nature, as distinguished from mutant forms that may result from selective breeding or transformation with a transgene.

As used herein, “identity,” is a relationship between two or more polypeptide or polynucleotide sequences, as determined by comparing the sequences. In the art, “identity” also refers to the degree of sequence relatedness between polypeptide as determined by the match between strings of such sequences. “Identity” can be readily calculated by known methods, including, but not limited to, those described in Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch (J. Mol. Biol., 1970, 48: 443-453) algorithm (e.g., NBLAST, and XBLAST). The default parameters are used to determine the identity for the polypeptides or polynucleotides of the present disclosure.

As used herein, “heterologous” refers to compounds, molecules, nucleotide sequences (including genes), and polypeptide sequences (including peptides and proteins) that are different in both activity (function) and sequence or chemical structure. As used herein, “heterologous” can also refer to a gene or gene product that is from a different organism. For example, a human GTP cyclohydrolase or a synthase can be said to be heterologous when expressed in yeast.

As used herein, “homologue” refers to a polypeptide sequence that shares a threshold level of similarity and/or identity as determined by alignment of matching amino acids. Two or more polypeptides determined to be homologues are said to be homologues. Homology is a qualitative term that describes the relationship between polypeptide sequences that is based upon the quantitative similarity.

As used herein, “paralog” refers to a homologue produced via gene duplication of a gene. In other words, paralogs are homologues that result from divergent evolution from a common ancestral gene.

As used herein, “orthologues” refers to homologues produced by speciation followed by divergence of sequence but not activity in separate species. When speciation follows duplication and one homologue sorts with one species and the other copy sorts with the other species, subsequent divergence of the duplicated sequence is associated with one or the other species. Such species specific homologues are referred to herein as orthologues.

As used herein, “xenologs” are homologues resulting from horizontal gene transfer.

As used herein, “similarity” is a quantitative term that defines the degree of sequence match between two compared polypeptide sequences.

As used herein, “cell,” “cell line,” and “cell culture” include progeny. It is also understood that all progeny may not be precisely identical in DNA content, due to deliberate or inadvertent mutations. Variant progeny that have the same function or biological property, as screened for in the originally transformed cell, are included.

As used herein, “culturing” refers to maintaining cells under conditions in which they can proliferate and avoid senescence as a group of cells. “Culturing” can also include conditions in which the cells also or alternatively differentiate.

As used herein, “organism”, “host”, and “subject” refers to any living entity comprised of at least one cell. A living organism can be as simple as, for example, a single isolated eukaryotic cell or cultured cell or cell line, or as complex as a mammal, including a human being, and animals (e.g., arthropods, vertebrates, amphibians, fish, mammals, e.g., cats, dogs, horses, pigs, cows, sheep, rodents, rabbits, squirrels, bears, primates (e.g., chimpanzees, gorillas, and humans). “Subject” may also be a cell, a population of cells, a tissue, an organ, or an organism, preferably to human and constituents thereof.

As used herein, “gene” refers to a hereditary unit corresponding to a sequence of DNA that occupies a specific location on a chromosome and that contains the genetic instruction for a characteristic(s) or trait(s) in an organism. As used herein, “synthetic gene” can refer to a recombinant gene comprising one or more coding sequences for a protein of interest, or a synthetically purified protein that is not naturally occurring in its purified state.

As used herein, the term “recombinant” generally refers to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc.). Recombinant also refers to the polypeptide encoded by the recombinant nucleic acid. Non-naturally occurring nucleic acids or polypeptides include nucleic acids and polypeptides modified by man.

As used herein, “plasmid” as used herein refers to a non-chromosomal double-stranded DNA sequence including an intact “replicon” such that the plasmid is replicated in a host cell.

As used herein, the term “vector” or “expression vector” is used in reference to a vehicle used to introduce an exogenous nucleic acid sequence into a cell. A vector may include a DNA molecule, linear or circular (e.g. plasmids), which includes a segment encoding a polypeptide of interest operatively linked to additional segments that provide for its transcription and translation upon introduction into a host cell or host cell organelles. Such additional segments may include promoter and terminator sequences, and may also include one or more origins of replication, one or more selectable markers, an enhancer, a polyadenylation signal, etc. Expression vectors are generally derived from yeast or bacterial genomic or plasmid DNA, or viral DNA, or may contain elements of both. As one of skill in the art would understand, recombinant coding sequences as described herein, for example any of the nucleotide sequences of (or nucleotide sequences encoding peptide sequences of) SEQ ID NOs: 1-24, can be cloned into a host-appropriate expression vector (also referred to herein as a backbone, using molecular cloning methods as known in the art) that is suitable for driving expression of the desired coding sequence (for example any of SEQ ID NOs: 1-24, or recombinant sequences encoding the peptide sequences of any of the nucleotide sequences of SEQ ID NOs: 1-24) in a desired host under the control of appropriate genetic elements (promoters and terminators, which can readily be determined by the skilled artisan according to desired expression levels in said host). An example of such a suitable vector which can be used as a host expression vector to express sequences as described herein is plasmid pBBR1MCS-2, available from Addgene®, and coding sequences can be cloned into the vector as a HindIII-BamHI insert, for example. Other examples of suitable vectors include a pUC backbone cloning vector, or a pMAL vector (available from New England Biolabs®). Further examples include vectors for the transformation of Bacilli such as pNW33n (available from the Bacillus Genetic Stock Center, BGSC). Furthermore, it is noted that expression vectors can contain suitable constitutive or inducible promotors to drive expressing of coding sequences as described herein, examples of promoters being known in the art and described herein.

As used herein, “operatively linked” or “operatively coupled” indicates that the regulatory sequences useful for expression of the coding sequences of a nucleic acid are placed in the nucleic acid molecule in the appropriate positions relative to the coding sequence so as to effect expression of the coding sequence. This same definition can also be applied to the arrangement of coding sequences and transcription control elements (e.g. promoters, enhancers, and termination elements), and/or selectable markers in an expression vector.

As used herein, “cDNA” refers to a DNA sequence that is complementary to a RNA transcript in a cell. It is a man-made molecule. Typically, cDNA is made in vitro by an enzyme called reverse-transcriptase using RNA transcripts as templates.

As used herein, the term “transfection” refers to the introduction of an exogenous and/or recombinant nucleic acid sequence into the interior of a membrane enclosed space of a living cell, including introduction of the nucleic acid sequence into the cytosol of a cell as well as the interior space of a mitochondria, nucleus, or chloroplast. The nucleic acid may be in the form of naked DNA or RNA, it may be associated with various proteins or regulatory elements (e.g., a promoter and/or signal element), or the nucleic acid may be incorporated into a vector or a chromosome.

As used herein, “transformation” or “transformed” refers to the introduction of a nucleic acid (e.g., DNA or RNA) into cells in such a way as to allow expression of the coding portions of the introduced nucleic acid.

As used herein, “stable expression,” “stable incorporation,” “stable transfection” and the like refer to the integration of an exogenous gene into the genome of a host cell, which can allow for long term expression of the exogenous gene.

As used herein, “transient expression,” “transient transfection,” and the like refer to the introduction of an exogenous gene into a host cell that does not result in stable incorporation of the gene into the host cell.

As used herein “chemical” refers to any molecule, compound, particle, or other substance that can be a substrate for an enzyme in the enzymatic pathway described herein and/or a carboxylesterase enzyme or biochemical pathway. A “chemical” can also be used to refer to a metabolite of a carboxylic ester. As such, “chemical” can refer to nucleic acids, proteins, organic compounds, inorganic compounds, metabolites etc. “Chemical” can also refer to the product produced by the carboxylesterase action.

As used herein “biologically coupled” refers to the association of or interaction between two or more physically distinct molecules, groups of molecules compounds, organisms, or particles where the association is directly or indirectly mediated between the two or more physically distinct molecules, groups of molecules compounds, organisms or particles via a biologic molecule or compound. This can include direct binding between two biologic molecules and signal transduction pathways.

As used herein, “biological communication” refers to the communication between two or more molecules, compounds, or objects that is mediated by a biologic molecule or biologic interaction.

As used herein, “biologic molecule,” “biomolecule,” and the like refer to any molecule that is present in a living organism and includes without limitation, macromolecules (e.g. proteins, polysaccharides, lipids, and nucleic acids) as well as small molecules (e.g. metabolites and other products produced by a living organism).

As used herein, “regulation” refers to the control of gene or protein expression or function.

As used herein, “promoter” refers to the DNA sequence(s) that control or otherwise modify transcription of a gene and can include binding sites for transcription factors, RNA polymerases, and other biomolecules and substances (e.g. inorganic compounds) that can influence transcription of a gene by interaction with the promoter. Typically these sequences are located at the 5′ end of the sense strand of the gene, but can be located anywhere in the genome.

As used herein, “native” refers to the endogenous version of a molecule or compound relative to the host cell or population being described.

As used herein, “non-naturally occurring” refers to a non-native version of a molecule or compound or non-native expression or presence of a molecule or compound within a host cell or other composition. This can include where a native molecule or compound is influenced to be expressed or present at a different location within a host, at a non-native period of time within a host, or is otherwise in an altered environment, even when considered within the host. Non-limiting examples include where a protein that is expressed only in the nucleus of a cell is expressed in the cytoplasm of the cell or when a protein that is only normally expressed during the embryonic stage of development is expressed during the adult stage.

As used herein, “encode” refers to the biologic phenomena of transcribing DNA into an RNA that, in some cases, can be translated into a protein product. As such, when a protein is said herein to be encoded by a particular nucleotide sequence, it is to be understood that this refers to this biologic relationship between DNA and protein. It is well established that RNA can be translated into protein based on the triplet code where 3 nucleotides represent an amino acid. This term also includes the idea that DNA can be transcribed into RNA molecules with biologic functions, such as ribozymes and interfering RNA species. As such, when a RNA molecule is said to be encoded by a particular nucleotide sequence it is to be understood that this is referring to the transcriptional relationship between the DNA and RNA species in question. As such “encoding nucleotide” refers to herein as the nucleotide which can give rise through transcription, and in the case of proteins, translation a functional RNA or protein.

As used herein “codon optimized” or “codon optimization” refers to a codon modification or making modifications to the codons for amino acids in a polypeptide such that they reflect the codon usage bias of the cell type that the polypeptide is expressed in. Modifications to the codons can be made using techniques generally known in the art.

As used herein, “derived” or “derived from” means copied from or modified from a starting material to a sequence or composition that is different than the starting material (not exactly the same as but retaining characteristics of, in terms of sequence, structure, concentration, quantity, etc), while maintaining the desired biological activity (for example enzymatic activity, nutritional function, etc). For example, a sequence that is derived from a parent sequence maintains sequences necessary for encoding or expression of a nucleotide that confers a biological function (such as enzymatic activity), but is altered in a way that the overall sequence of the derived sequence is not exactly identical to the parent sequence.

### Discussion

Described herein are compositions and methods relating to pesticide degradation. Compositions and methods relating to pesticide degradation can comprise the exogenous expression of one or more recombinant carboxylesterase enzymes, oxalate decarboxylase enzymes, and/or one or more enzymes related to neonicotinoid (or neonicotinoid metabolite, such as 6-chloronicotonic acid, 6-CNA) degradation.

Compositions and methods as described herein serve as improvements upon existing technologies in the state of the art, in part, by application of enzymatic remediation strategies to the hive environment (for example the hive environment of a bee of the Apis or Bombus genus) and modification of the bee gut microbiota. Such application of remediation strategies to the hive environment in particular is a practical application that provides pollinator insects with defenses against commonly used pesticides, the effects of which on pollinator insects have been devastating to pollinator colonies residing in such hives.

In additional aspects, such bacteria can also be selected and amplified from the milieu of the hive microorganisms and in some cases they can be molecularly bred to enhance their metabolic capabilities without genetic engineering and reintroduced to the hive at a level or amount significantly greater than the natural-occurring level or amount.

Compositions and methods as described herein comprise recombinant carboxylesterase enzymes, oxalate decarboxylase enzymes, or recombinant neonicotinoid (or neonicotinoid metabolite) degrading enzymes. Recombinant carboxylesterase enzymes, oxalate decarboxylase enzymes, or neonicotinoid (or neonicotinoid metabolite) degrading enzymes can be expressed in engineered cells as described herein, can be purified and introduced into or otherwise mixed with bee bread (synthetic or wild-type), or both. Such engineered cells can also be added to the hive directly. In certain aspects, recombinant carboxylesterase enzymes, oxalate decarboxylase enzymes, or neonicotinoid (or neonicotinoid metabolite) degrading enzymes are present in the engineered cell[s], the bee bread, or both in an amount effective to reduce the active ingredient of an insectide (for example a pyrethroid, synthetic pyrethroid, organophosphate, or neonicotinoid) from a first level to a second level, where the second level is less than the first level.

Recombinant carboxylesterase enzymes as described herein are enzymes that catalyze the hydrolysis of carboxylic esters. In certain aspects, recombinant carboxylases as described herein are derived from insects. In certain aspects, recombinant carboxylases as described herein are derived from thermophilic bacteria.

In certain embodiments as described herein, a recombinant carboxylesterase can be Methyl Parathion Hydrolase (MPH), Pyrethroid Hydrolase (PytH), or E3 carboxylesterase, or derivatives there.

In an embodiment, a carboxylesterase as described herein is Methyl Parathion Hydrolase (MPH), an OP carboxylesterase, originally isolated from Plesiomonas sp. M6 (33), GenBank accession No DQ677027).

In an embodiment, a carboxylesterase as described herein is Pyrethroid Hydrolase (PytH) (GenBank accession No FJ688006), encoded by the PytH gene from Sphingobium sp. JZ-1.

In an embodiment, a carboxylesterase as described herein is a naturally-occurring variant (Trp251Leu) of the E3 carboxylesterase (wild-type allele GenBank: AAB67728) from the blowfly Lucilia cuprina.

In an embodiment, a carboxylesterase as described herein is Deinococcus radiodurans phosphotriesterase (Genbank accession #3GTF_A). In an embodiment, a carboxylesterase is Sulpholobus acidocaldarius phosphotriesterase (Genbank accession #WP_011278935).

Embodiments of nucleotide coding sequences of recombinant carboxylesterases as described herein are shown in the examples below, for example in Example 3 and Example 4 below. Recombinant carboxylesterases as described herein can be codon-optimized according to the host in which they are to be expressed. Furthermore, in certain aspects, recombinant carboxylesterases can be have coding sequences that retain sequence identify of about 90% to about 100% to embodiments of sequences as described herein, for example SEQ. ID. NOs 1-5.

Recombinant oxalate decarboxylase (ODC) enzymes as described herein are enzymes are manganese-containing, multimeric enzymes of the cupin protein superfamily. ODC is one of the three enzymes identified to decompose oxalic acid and oxalate, and within ODC catalysis, oxalate is split into formate and CO₂. In certain aspects, recombinant oxalate decarboxylases as described herein are derived from fungi and/or bacteria. In certain aspects, recombinant carboxylases as described herein are derived from thermophilic bacteria.

In certain embodiments as described herein, a recombinant oxalate decarboxylase can be B. subtilis, Burkholderia pseudomallei, or Pantoea allii oxalate decarboxylase, or derivatives thereof.

Embodiments of nucleotide coding sequences of recombinant oxalate decarboxylases as described herein are shown in the examples below, for example in Example 7 and 9 below. Recombinant oxalate decarboxylases as described herein can be codon-optimized according to the host in which they are to be expressed. Furthermore, in certain aspects, recombinant oxalate decarboxylases can be have coding sequences that retain sequence identify of about 90% to about 100% to embodiments of sequences as described herein, for example SEQ. ID. NOs 6-11.

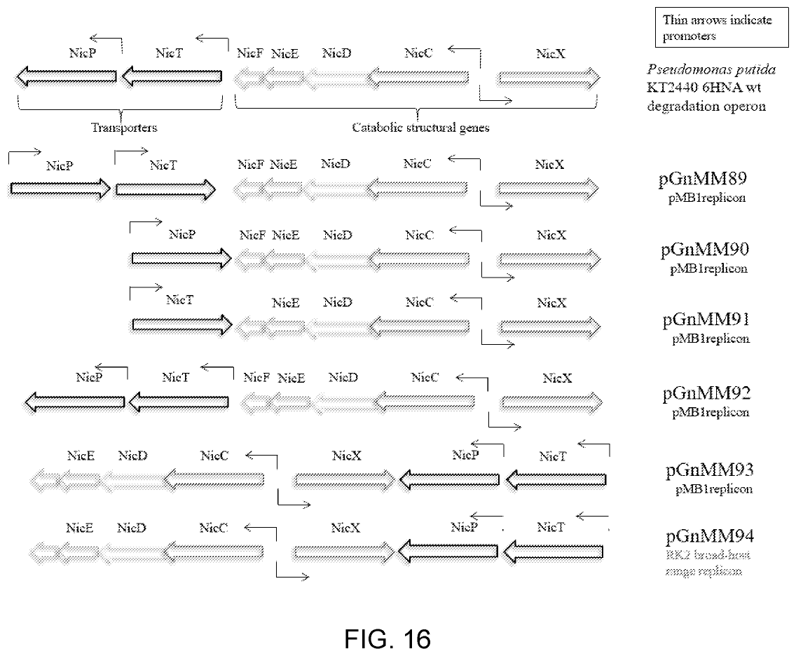

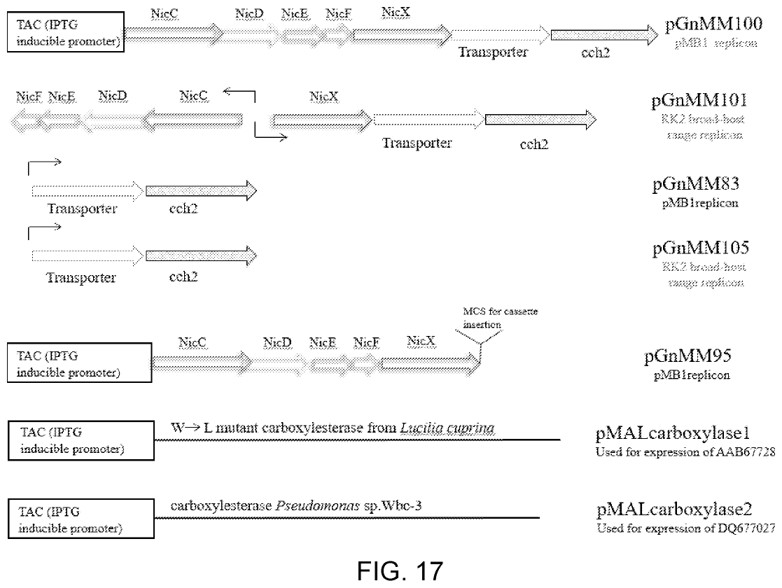

Examples of neonicotinoid (or neonicotinoid metabolite, for example 6-CNA) degrading enzymes are described, for example, in Example 5 and FIGS. 16-17. In certain embodiments, an expression vector expresses a combination of a plurality of these enzymes (for example at least 6-chloronicotinic acid chlorohydrolase, also known as cch2, coding sequences of which are known in the art). In certain aspects, neonicotinoid degradation can be facilitated by the expression of a recombinant cch2 enzyme (with or without transporter or other signal sequence), and combination with the expression (endogenous or exogenous) of a functional 6-HNA degradation pathway, for example, such as the one which is active in the Pseudo monas putida KT2440, and comprises nucleotide sequences encoding coding sequences of SEQ ID NOs: 14-24.

Described herein are expression vectors for the expression of recombinant carboxylesterases or neonicotinoid (or neonicotinoid metabolite) degrading enzymes as described herein. In certain embodiments, expression vectors as described herein are plasmid expression vectors comprising a coding sequence for one or more recombinant carboxylesterases or neonicotinoid (or neonicotinoid metabolite) degrading enzymes as described herein. Furthermore, in certain aspects, recombinant neonicotinoid (or neonicotinoid metabolite) degrading enzymes can be have coding sequences that retain sequence identify of about 90% to about 100% to embodiments of cch2 sequences as described herein, for example SEQ. ID. NOs 12-13.

In certain embodiments, coding sequences and/or expression vectors for the expression of recombinant carboxylesterases or neonicotinoid (or neonicotinoid metabolite) degrading enzymes can also comprise a coding sequence for a signal peptide for enzyme secretion. In an embodiment, a recombinant cch2 is fused with a sequence encoding a transport sequence (at either the 5′ or 3′ end of the encoding mRNA or N- or C-terminus of the resultant peptide). Such examples are known in the art.

Described herein are engineered cells. Engineered cells as described herein can be gram positive (+) or gram negative bacteria (−). Engineered cells as described herein can comprise expression vectors for the expression of recombinant carboxylases or neonicotinoid (or neonicotinoid metabolite) degrading enzymes as described herein. Engineered cells can express exogenous recombinant carboxylesterases (in other words, express recombinant carboxylesterases that are not expressed under wild-type normal physiological conditions), or can otherwise express recombinant carboxylesterases or neonicotinoid (or neonicotinoid metabolite) degrading enzymes at a level above normal wild-type physiological expression. Engineered cells can be engineered with expression vectors comprising one or more coding sequences for recombinant carboxylesterases or neonicotinoid (or neonicotinoid metabolite) degrading enzymes according to known methods of the art, for example transformation by electroporation and/or conjugation.

In certain aspects, engineered cells as described herein are derived from bacteria of the genus Lactobacillus. In an embodiment, an engineered cell as described herein can be a derivative of the species Lactobacillus kunkeei, L. lactis, L. kullabergensis, L. melliventris, L. kimbladii, L. helsinborgensis, L. mellis, L. sp. AcjLac18.

In certain aspects, engineered cells as described herein are derived from bacteria of the genus Fructobacillus. In an embodiment, an engineered cell as described herein can be a derivative of the species Fructobacillus fructosus.

In certain aspects, engineered cells as described herein are derived from bacteria of the genus Gilliamella. In an embodiment, an engineered cell as described herein can be a derivative of the species Gilliamella apicola.

In certain aspects, engineered cells as described herein are derived from bacteria of the genus Snodgrassella. In an embodiment, an engineered cell as described herein can be a derivative of the species Snodgrassella alvi.

In certain aspects, engineered cells as described herein are derived from bacteria of the genus Actinobacteria. In an embodiment, an engineered cell as described herein can be a derivative of the species Bifidobacterium asteroides or B. coryneforme.

In certain aspects, engineered cells as described herein are derived from bacteria of the genus Parasaccharibacter. In an embodiment, an engineered cell as described herein can be a derivative of the species Parasaccharibacter apium.

In certain aspects, engineered cells as described herein are derived from bacteria of the genus Bacillus. In an embodiment, an engineered cell as described herein can be a derivative of the Bacillus subtilis group (Bacillus subtilis, Bacillus amyloliquefaciens, Bacillus velezensis), Bacillus megaterium, or Bacillus licheniformis. In an embodiment expression of the aforementioned enzymes is intracellular, in another embodiment expression can be extracellular, in a further embodiment, the gene(s) of interest is expressed in the spores or on their surface.

In certain aspects, engineered cells as described herein are derived from bacteria of the genus Pseudomonas, Stenotrophomonas, and/or Sphigobacterium.

Also described herein are modified bee breads. Modified bee breads can comprise bee bread, pollen and carbohydrates, or pollen and carbohydrates; and one or more purified recombinant carboxylesterases or neonicotinoid (or neonicotinoid metabolite) degrading enzymes according to the present disclosure, one or more engineered cells according to the present disclosure, or both. The bee bread can be wild-type bee bread. The bee bread can be synthetic bee bread. The one or more purified recombinant carboxlyesterases can be Methyl Parathion Hydrolase (MPH), Pyrethroid Hydrolase (PytH), E3 carboxylesterase, E3^{Trp251Leu} carboxylesterase, Deinococcus radiodurans phosphotriesterase, or Sulpholobus acidocaldarius phosphotriesterase. The modified bee bread can contain one or more purified recombinant carboxylesterases or neonicotinoid (or neonicotinoid metabolite) degrading enzymes present in an amount effective to reduce the amount of active ingredient of one or more insecticides from a first level to a second level, wherein the second level is less than the first. The modified bee bread can comprise one or more engineered cells present in an amount effective to reduce the amount of active ingredient of one or more insecticides from a first level to a second level, wherein the second level is less than the first. The one or more insecticides are one or more insecticides from the pyrethroid, synthetic pyrethroid, organophosphate, or neonicotinoid class of insecticides.

Described herein are methods of conferring protection from insecticides to one or more subjects. Methods as described herein can comprise providing a bee bread; providing one or more purified recombinant carboxylesterases or neonicotinoid (or neonicotinoid metabolite) degrading enzymes according to the present disclosure, one or more engineered cells according to the present disclosure, or both; and introducing the one or more purified recombinant carboxylesterases or neonicotinoid (or neonicotinoid metabolite) degrading enzymes according to the present disclosure, one or more engineered cells according to the present disclosure, or both into the bee bread. The bee bread according to methods of the present disclosure can be wild-type bee bread. The bee bread according to methods of the present disclosure can be synthetic bee bread (comprising about 5% to about 55% pollen and about 5% to about 25% sugar, such as fructose). One or more purified recombinant carboxlyesterases according to the present disclosure can be Methyl Parathion Hydrolase (MPH), Pyrethroid Hydrolase (PytH), E3 carboxylesterase, E3^{Trp251Leu} carboxylesterase, Deinococcus radiodurans phosphotriesterase, or Sulpholobus acidocaldarius phosphotriesterase. One or more carboxylesterases or neonicotinoid (or neonicotinoid metabolite) degrading enzymes according to methods of the present disclosure can be present in an amount effective to reduce the amount of active ingredient of one or more insecticides from a first level to a second level, wherein the second level is less than the first level. One or more engineered cells according to methods of the present disclosure can be present in an amount effective to reduce the amount of active ingredient one or more insecticides from a first level to a second level, wherein the second level is less than the first level.

Also described herein are methods of creating an engineered cell. Methods of creating an engineered cell as described herein can comprise providing wild-type bee bread; isolating one or more bacteria from the wild-type bee bread; introducing one or more expression vectors comprising one or more recombinant carboxylesterase or neonicotinoid (or neonicotinoid metabolite) degrading enzyme coding sequences into the one or more bacteria; selecting one or more stably-expressing recombinant carboxylesterase or neonicotinoid (or neonicotinoid metabolite) degrading enzyme bacteria after the one or more expression vectors are introduced; and isolating the stably-expressing recombinant carboxylesterase or neonicotinoid (or neonicotinoid metabolite) degrading enzyme bacteria.

Further described herein are engineered cells created by any of the methods as described herein.

Also described herein are methods of treating a subject for pesticide exposure. Methods of treating a subject for pesticide exposure can comprise delivering a modified bee bread according to the present disclosure to a subject in need thereof. The modified bee bread can be delivered to the subject in an amount effective to reduce the active amount of one or more insecticides from a first level to a second level, wherein the second level is less than the first. The one or more insecticides according to methods of treatment as described herein can be one or more insecticides from the pyrethroid, synthetic pyrethroid, organophosphate class of insecticides, or neonicotinoid class of insecticides. A “subject in need thereof” according to methods of treatment of the present disclosure can be a subject as described herein (a bee of the Apis or Bombus genus) that is subjected to, or at risk for, pesticide exposure. In certain embodiments, a “subject in need thereof” is a honey bee that is subjected to, or at risk for, pesticide exposure.

Kits

Also provided herein are kits containing a cell or population thereof (homogeneous or heterogeneous) which are to express one or more recombinant carboxylases, oxalate decarboxylases, or neonicotinoid (or neonicotinoid metabolite) degrading enzymes as described herein, and/or one or more vectors configured to express one or more recombinant carboxylases, oxalate decarboxylases, or neonicotinoid (or neonicotinoid metabolite) degrading enzymes as described herein. In an embodiment, one or more vectors configured to express one or more recombinant carboxylases oxalate decarboxylases, or neonicotinoid (or neonicotinoid metabolite) degrading enzymes as described herein can comprise one or more of SEQ ID NOs: 1-24 (or, if the SEQ ID. referred to is a peptide sequence, a recombinant nucleotide sequence encoding the referenced peptide sequence). The kit can contain one or more substrates suitable for use with the engineered cells as described herein. The kit can further contain additional reagents, diluents, cell culture media, cell culture plates or other container, syringes, and other components (cells, vectors, transfection regents, etc.) that can be used with the a cell or population thereof and/or one or more vectors as described herein.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

Insect pollination is vital to terrestrial ecosystems and to crop production. First-line stakeholders include the farming industry, agri-food processors, and ultimately, the society at large. Although most calories humans gain from food are derived from wind-pollinated crop plants, it is estimated that about ¾ of cultivated plant species benefit from insect pollinators (77). The added value of pollination by honey bees alone is in the tens of billions of dollars worldwide (50, 23, 147). According to the 2017 USDA Annual Report, honey bees “add at least $15 billion to the value of U.S. agriculture annually through increased yields and superior-quality harvests” (1, 2). Wild pollinators (insects, birds, and some mammals), also substantially contribute to pollination, and in some cases, these may outperform honey bees (118, 113). Wild insect pollinators and honey bee colonies, both managed and feral, are currently experiencing high gross loss rates in North America and Europe (58). These losses depend on the type of operation, location, and year, and average between 20-30% annually (146, 3, 4). According to The Bee Informed Partnership, a non-profit organization connected to academic institutions, winter and summer combined losses currently range between 35-45% (6). The total cost of colony losses, the cost for their replacement and the loss of income combined, are likely to exceed hundreds of million annually (5).

The consensus is that honey bee colony losses are not linked to any single factor. Rather, honey bee decline can be due to the combined effect of several stressors that can be grouped into multiple categories (58): 1) malnutrition; 2) parasites and pathogens, especially the parasitic mite Varroa destructor and its associated pathogens; 3) queen quality and 4) sublethal exposure to pesticides, the focus of this proposal. Described herein are compositions, methods, and strategies to mitigate or overcome insecticide poisoning by exploiting a novel application of paratransgenesis.

As described herein, pollen contaminated by insecticides can be assimilated to a sludge in need of bioremediation, thus allowing the application of strategies and technologies developed over decades of environmental biotechnology. More specifically, by genetically engineering bee bread-adapted bacteria capable of degrading at least two classes of insecticides, organophosphates (OP) and synthetic pyrethroids (SP), individually or in combination, a safer environment for honey bees can be created. Furthermore applying the same genetic modifications to bacteria which are considered honey bee symbionts sensu stricto, a synergistic layer of protection can be bestowed upon honey bees. Compositions, methods, and strategies as described herein can entail the genetic manipulation of honey bee commensals to degrade OP and SP insecticides using insecticide-degrading carboxylesterases derived from soil and water-borne bacteria, (34, 71, 139). Compositions and methods as described herein can provide protection to honey bees against OP and SP insecticides.

Compositions and methods as described herein can involve the expression of a panel of pyrethroid and organophosphate degrading enzymes with broad-range activities in honey bees commensals and sensu stricto symbionts. In certain aspects, a single degradation enzyme can be expressed, in other aspects, more than one (ie a plurality) may be expressed.

Further described herein are bacteria, and their combinations, that can confer to honey bees protection against pesticides.

Last, described herein is the engineering of antibiotic resistance-free, food-grade genetically modified commensals that can stably express one or more enzymes needed for pesticide remediation.

Wild and managed pollinator declines are, in part, due to nutrition stress, pests, pathogens, queen quality, and exposure to agrochemicals. The occurrence and combination of these stressors vary in time and location (134, 51, 167), thus making it difficult to quantify the relative contribution each has to honey bee losses. While each of these stressors alone poses a significant economic impact, their combination is even more problematic (135, 84, 167).

Honey bees are intimately connected to their macro- and microbiota, similar to the relationship between mammals and their microbiomes. Upon closer examination, the honey bee colony's microbiome revealed similarities to those of mammals. Namely, the hive's microbiome provides both micro- and macro-nutrients; it stabilizes the mixture of pollen and nectar by converting these into bee bread (86, 110, 124, 52, 13, 85); and it maintains optimal insect health (14, 9, 49, 30, 15, 83). Similar to mammals, variations in the microbiome composition contribute to pathological conditions, although it is unclear if such changes are considered as the cause or an effect of Colony Collapse Disorder (CCD) (169). The relevance of the bee microbiome in maintaining adequate hive health has become a focus of intense research (7, 82).

A paratransgenic perspective of the honey bee microbiome. Paratransgenesis is an approach whereby members of the commensal community are genetically modified in lieu of the host. Currently, the honey bee microbiota is described in terms of a “core-microbiome,” which, according to studies based upon 16S rDNA sequencing, is composed by a set of nine niche-adapted bacterial species and clades of very closely related bacteria. (82). Several members of the proposed core microbiome are shared across honey bee subspecies and across continents, with some members being shared between corbiculate bees of the genera Apis and Bombus (149, 97, 12). Some investigators found only partial support for a “core microbiome” model (101, 12, 66, 90). An investigation of the metabolically active honey bee microbial community through the analysis of 16S rRNA proposes a more complex microbiome (99), but there is debate as to what approach best reflects the microbiome (127). The honey stomach harbors a different microflora than does the rest of the intestine (29); therefore, considering it a part of the gut or an external appendix influences the overall composition of the microbiome. For the purpose of the present disclosure, an exact distinction between the honey bees' commensals, straddling the line between the honey bee stomach and the hive food stores, and the symbionts sensu stricto is not needed.

The selection of paratransgenesis candidates. Regardless of the exact composition of the core microbiome, it may be impractical to propose a paratransgenesis strategy targeting all of its members. The modification of the entire microflora is also not necessary, in fact, the addition of one or very few microorganisms ameliorated Irritable Bowel Syndrome (IBS), diabetes, rheumatoid arthritis, and atopic dermatitis (100, 94, 128, 143, 160, 56). Disclosed herein are compositions and methods to enrich the honey bee microflora stratified with insecticide-degrading commensal bacteria to provide protection against insecticide-mediated poisoning. Honey bees can then be challenged with insecticides, and their tolerance can be compared to that of honey bees carrying unmodified microflora in order to evaluate the efficacy of compositions and methods as disclosed herein. It is believed that the honey bee stomach, also known as crop or foregut, and its associated microflora, can be relevant to the scope of this proposal. The current knowledge of the bee bread microbiology is based on a 50 year-old seminal work (110) that has recently been refined (12). Pollen contains its own microflora including lactic acid bacteria (LAB). Upon collection by honey bees, pollen is mixed with the regurgitated contents of the honey stomach, and begins the fermentation process that ultimately can contribute to the microbiological stability of the bee bread. Although the relative contribution of different bacteria is open to investigation, the microbiological transformation from pollen to bee bread needs a bacterial consortium to be fully palatable to honey bees (110). During this conversion process, the varied microflora of the pollen can progressively disappear while LAB rise in numbers and then fade away (part of this process is illustrated by the data of FIGS. 6A-6B). In fact, simulated bee bread fermented with recombinant LAB as described herein can remain microbiologically stable and palatable to honey bees for months. The honey stomach is the honeybees' nutritional interface with the environment, food stores, and other members of the colony including the queen and the larvae. Therefore, the associated microorganisms such as Lactobacillus kunkeei (L. kunkeei) and Fructobacillus fructosus (F. fructosus) (150, 12; 29, 93) can be deployed as a first-line defense against toxic xenobiotics. Besides being the gatekeeper of the gut, the honey stomach is the organ where the fermentation of bee bread begins; therefore the presence of insecticide-degrading bacteria in the honey stomach can be beneficial in that, ultimately, they can detoxify the food stores where insecticides accumulate. Moreover, some of the microbiota of the honey stomach, also represent a substantial fraction of young larvae microbiota (29, 30), thus potentially being capable of extending protection against polluting insecticides outside the worker bee caste.

The gut deep compartments, e.g., midgut, ileum, and rectum, are colonized by different bacteria than those that colonize the honey stomach and because microaerophilic, O₂-tolerant bacteria become predominant. The Gram-positive LAB, which inhabit the O₂-limited portions of the gut, are often described as two Operational Taxonomic Units (OTUs), named Firm-4 and Firm-5. Some assign species names to individual members of these two clades (107). For the very nature of this investigation, bacteria can be dealt with as single species as opposed to metadata categories. Accordingly, a binomial nomenclature is adopted herein, as detailed below. Two Gram-negative bacteria, Gilliamella apicola and Snodgrassella alvi, as well as a few species of Actinobacteria, mainly Bifidobacterium asteroides and B. coryneforme, are consistently found to dwell in the gut deep compartments. Members of each of the aforementioned groups have been isolated. While all the members of the honey bees' microflora may not have been isolated, a panel of honey bee commensals which are known to inhabit the gut have been isolated and investigated herein, (98, 150, 12, 114).

A novel approach to paratransgenesis. Paratransgenesis is a “Trojan horse” approach that was originally designed to control insect-borne diseases. The overall strategy entails the isolation of symbiotic microorganisms that are indigenous to the disease-transmitting insects, and subsequently genetically manipulating these to produce molecules that would hinder disease transmission. Genetically modified symbionts are then reintroduced into their host-vector, where they can deliver effector proteins or peptides. Such studies have shown promise (43, 46, 67). Genetically engineered Enterobacter cloacae expressing an insecticidal toxin from Photorhabdus luminescens has been shown to kill termites in lab experiments (165). More recently, genetically engineered yeast (Kluyveromyces lactis) expressing a ligand-Hecate fusion peptide was used to kill termites by eliminating their gut protozoa which assist the termite workers with the digestion of cellulose (137). It has been postulated, however, that paratransgenesis, when deployed to control insects, may negatively impact the fitness of the host, thus limiting or preventing the dissemination of the transformed symbionts into the targeted population (32). Instead, the concept of paratransgenesis as described herein is expanded as a preventative and intervention therapy to improve honey bees fitness by abating the amount of some insecticides that pollute the hive super-organism. This novel application for paratransgenesis has been speculated as a possible method to improve honey bee health (119, 93, 120, 17), but its application has yet to be tested.

The target insecticides. Described herein are compositions and methods to increase honey bee tolerance to organophosphate (OP) and synthetic pyrethroid (SP) insecticides by enabling selected members of the honey bee microflora to degrade these toxic xenobiotics enzymatically. OPs are esters of phosphoric acid, and they negatively impact almost every biological system they contact. They constitute the basis for some nerve agents developed for chemical warfare, as well as for some widely used insecticides (139). The lethality of OPs ultimately results from their interaction with the active site of acetylcholinesterase (AChE), which prevents breakdown of the neurotransmitter acetylcholine, thus causing continuous nerve impulses at these synapses. Because of their poisonous effects, OPs tend to be strictly regulated. SPs are neurotoxins that are chemical analogues of pyrethrins, originally detected in Chrysanthemum spp. SPs target the voltage-gated sodium channels and impede their inactivation, resulting in persistent sodium inflow and leaving the axonal membrane permanently depolarized, causing paralysis (183). Although, SPs are considered to be safer than other insecticides, the common and pervasive use of these compounds in a wide variety of fields, including in honey bee colonies for Varroa control, has resulted in widespread contamination of the environment posing an ecological concern. The results of many studies have revealed that SPs negatively affect non-target organisms such as fish and arthropods, including honey bees (182, 186,). Moreover, chronic exposure to SPs appears to have long-term effects on human health that are far more serious than previously thought (34). Pyrethroids differ from many other pesticides in that they contain one to three chiral centres. As a result, pyrethroids can have two to eight enantiomers. The chiral isomers of SP differ from each other in their biological properties, including insecticidal activity and their sensitivity to enzymatic hydrolysis (183). Such characteristics can be taken into consideration in the choice of the enzyme(s) responsible for their degradation, as detailed below. Two compounds, one from both insecticide classes, are used by beekeepers for the control of Varroa in honey bee colonies, coumaphos (OP) and fluvalinate (SP). Widespread resistance of Varroa to these compounds have been reported consistently (175, 178, and references therein) rising concerns about the sustainability of these approach. Consequently, high residues of both compounds have been found in honey bee colonies (102). A third class of insecticides, the neonicotinoids, is capable of severely impacting pollinator health (69), but to what extent is debated. However, the knowledge regarding their degradation pathways remains incomplete, even though it is known that neonicotinoids can undergo microbial bioremediation in soil and water (68). The lack of such knowledge is relevant because several neonicotinoid intermediate metabolites are more toxic and persistent than the original pesticide (103, 104 140).

Three groups have established the technology to perform honey bee paratransgenesis (93, 120,177). The genetic transformation of L. kunkeei and F. fructosus, has been established using both theta and rolling-circle replicons. Green fluorescent protein and m-Cherry were successfully expressed into both species. A 7.3 Kb operon from Lactococcus lactis, encoding the 5 genes of the Leloir pathway for lactose utilization, in F. fructosus (93) has been reconstructed and functionally expressed. In order to expand the microbial toolbox herein, commensal LAB encompassing the OTUs Firm4 and Firm5 have been isolated. (L. kullabergensis, L. melliventris, L. kimbladii, L. helsinborgensis, L. mellis, L. sp. AcjLac18. Parasaccharibacter apium (Genbank MH205731) and S. alvi pAJ198 (Genbank MH744154 have also been isolated.

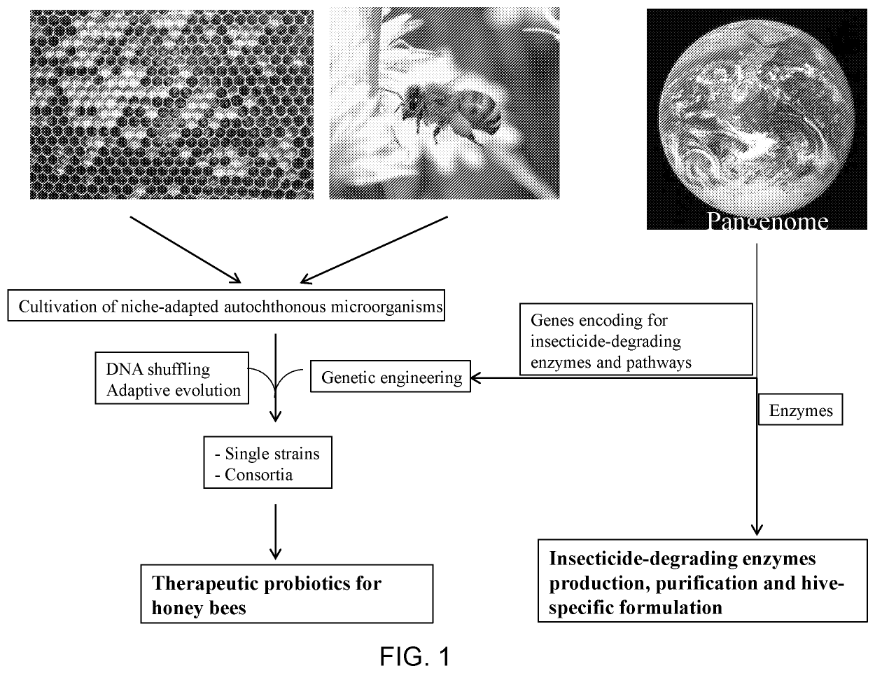

FIG. 1 is an overview of strategies for bioremediation of xenobiotics in the honey bee hive as discussed herein.

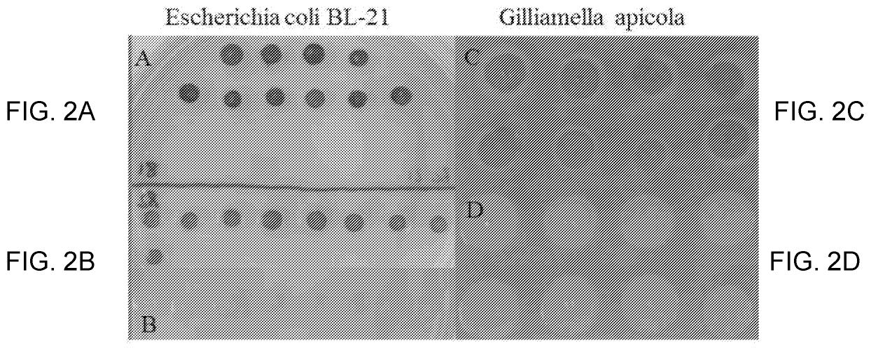

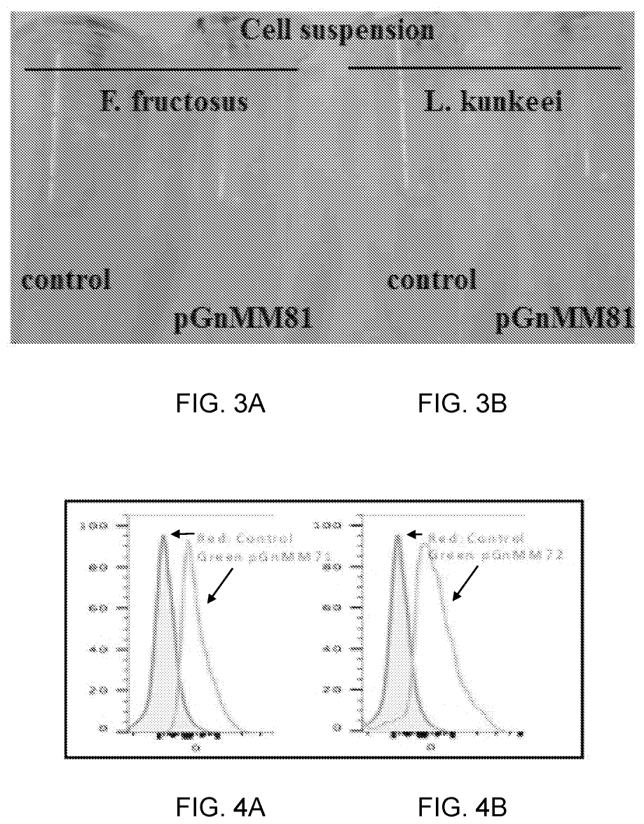

The expression of carboxylesterases in Gram− (FIGS. 2A-2D) and Gram+ (FIGS. 3A-3B) is shown herein.

FIGS. 2A and 2B are photographs depicting the expression of carboxylesterase in E. coli (FIG. 2A) and G. apicola (FIG. 2C). FIGS. 2B and 2D show the empty vector comtron. The chromogenic substrate X-caprylate is overlaid onto colonies.

FIGS. 3A-4B are photographs demonstrating expression of carboxylesterase in L. kunkeei (FIG. 3B) and F. fructosus (FIG. 3A). The chromogenic substrate is 4-nitrophenyl acetate added to the bacterial cell suspension.

Furthermore the capability to chromosomally engineer S. alvi, arguably, the most fastidious among honey bee commensals is shown in FIGS. 4A-4B. FIGS. 4A-4B represent flow cytometry data of S. alvi chromosomally transformed with two integrative vectors expressing m-Cherry.

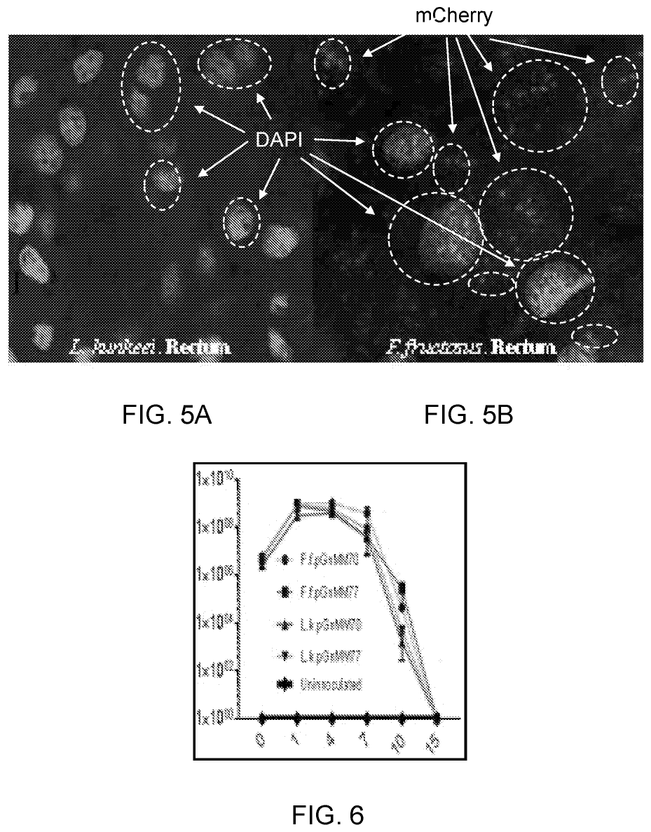

Confocal microscopy detected the insertion of bacteria into the honey bee gut and will for the evaluation of the gene-expression achieved with different constructs in vivo (FIGS. 5A-5B). FIGS. 5A-5B are confocal micrographs of guts of honey bees fed recombinant LAB bearing pGnMM70 expressing m-Cherry. This approach will enable to determine suitable bacteria-plasmid combinations. This construct is silent in L. kunkeei cells (FIG. 5A) which regain fluorescence when extracted from the rectum and plated on MRS+erythromycin (FIG. 5B). Bacteria are shown in red (m-Cherry expression), gut cell nuclei are shown in blue (4′,6-diamidino-2-phenylindole, DAPI) (see representative annotations).

In addition, it is shown that recombinant LABs expressing m-Cherry can grow 3 logs in high-pollen simulated bee bread (50% pollen/20% fructose) to very high-density, 109/ml, (FIG. 6). FIG. 6 is a graph depicting 3-log growth of m-Cherry transformed F. fructosus and L. kunkeei in high-pollen simulated bee bread. Transformed bacterial colonies remained Eryth^{Res} m-Cherry⁺ even if no antibiotic was present in the bee bread. pH decreased from 4.5 to 3.5. At the end no colonies detected even without erythromycin. Importantly, they maintained the recombinant plasmid because they tested Erythr^{Res} and m-Cherry+. At the end of the growth, no colonies could be detected even withdrawing erythromycin, thus confirming old observations suggesting that bee bread becomes virtually sterile (110). This observation is relevant to applications of compositions and methods as described herein because, even assuming that some decontaminating bacterial species or consortia may be harmful to honeybee when delivered outside the normal mechanisms of microflora transmission, the fermentation process will eventually clear the decontaminating microorganisms.

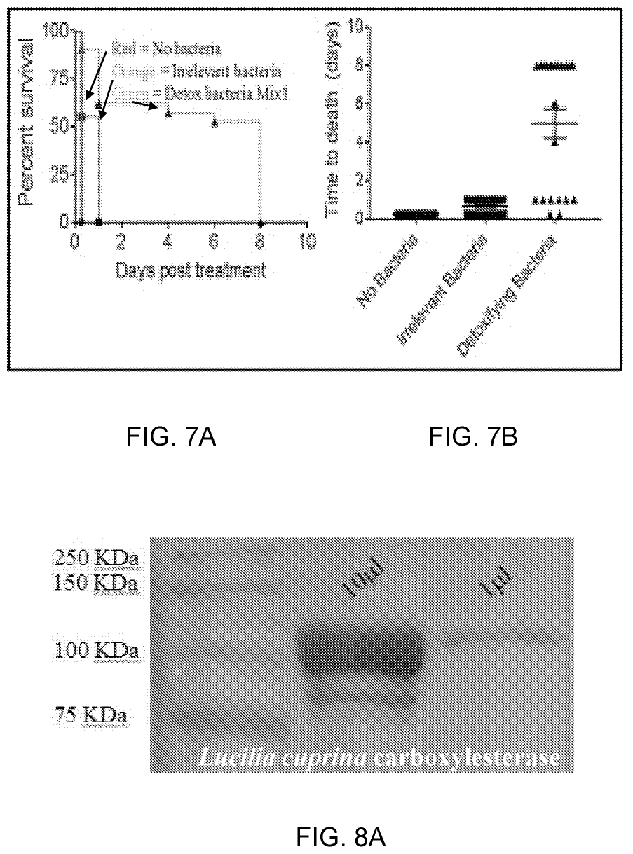

In order to prove the feasibility of an embodiment of a whole cell bioremediation strategy, a simulated bee bread was fermented with 1) no bacterial inoculum, 2) an inoculum of non-engineered bacteria, and 3) the corresponding mix of engineered bacteria. Mortality was then recorded and plotted (FIGS. 7A-7B). FIGS. 7A-7B are graphs depicting data from a 40xLC₅₀ cypermethrin challenge. Simulated bee bread was spiked with the highly toxic cypermethrin, fermented with a mix of selected bacteria and fed to the bees. Cypermethrin concentration was adjusted to deliver 40xLC₅₀ daily (assuming that each bee would take 25 ml of the simulated bee bread daily). Cypermethrin-spiked medium was not withdrawn at any time. FIG. 7A depicts a survival curve for the bacteria (percent survival), and FIG. 7B illustrates time to death (days). These data show that some of the recombinant bacteria can protect honey bees against a severe >40xLC50 persistent challenge with the highly toxic cypermethrin, exactly: 41.6xLD50 or 2.5 mg/bee/day (182), based on a projected 25 ml bee daily ingestion, delivered by simulated bee bread, thus being logarithmically higher that the sub-lethal concentrations expected in the field.

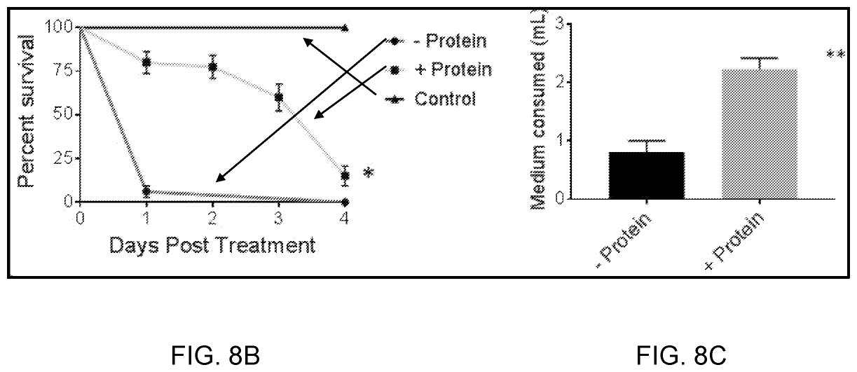

In order to demonstrate the feasibility of the cell-free enzymatic bioremediation, the carboxylesterase was recombinantly expressed in E. coli BL21 and purified (FIG. 8A is a Coomassie staining showing purified recombinant carboxylesterase). A 20% fructose syrup which was spiked with cypermethrin and remediated with the purified carboxylesterase (+protein) or left untreated (−protein). Syrups were fed to honey bees and mortality was recorded. A batch of bees was fed fructose syrup without any treatment (control) (FIGS. 8B-8C).

FIGS. 8B-8C illustrate data from a 40xLC₅₀ cypermethrin challenge. 20% fructose syrup was spiked with cypermethrin and bioremediated with the purified carboxylesterase. Cypermethrin concentration was adjusted to deliver 40xLD₅₀ daily (assuming that each bee would take 25 ml of the syrup daily). 10×5 bees were used in each batch. Cypermethrin-spiked syrup was not withdrawn at any time. Media consumption shows that bees kept feeding during the experiment. FIG. 8B illustrates a survival curve of bees without protein (−) (bottom; circles), with protein (+) (middle; squares), and control (top; triangles). FIG. 8C shows the amount of medium consumed without protein (−) and with protein (+).

An aspect of novelty resides in considering the hive stores as a substrate for whole cell biocatalysis and enzyme bioremediation. As an added layer of protection honey bee symbionts sensu stricto will also be targeted to increase protection.

The metabolic capabilities of microorganisms to degrade OPs and SPs are widespread in the environments where these compounds are present, and the horizontal transmission of DNA fragments encoding degrading enzymes is an occurrence in nature, with carboxylesterase resistance genes apparently travelling even across continents (125). While the possibility that insecticide-degrading resistance genes may spread from engineered honey bees' commensals exists, such horizontal gene transfers already do occur without consequences. An example of horizontal gene transfer is the spread of resistance genes to the antibiotics kanamycin and hygromycin, and herbicide-resistance genes transmitted between transgenic crops and soil microorganisms, both have been occurring for more than 20 years upon hundreds of million acres with no apparent detrimental effects (137, 126, 144). The requirement for antibiotic-free GMOs is not universally enforced because a substantial fraction of transgenic crops carry either kanamycin or hygromycin resistance genes. Nonetheless, modifications are disclosed herein that will minimally impact the overall gene-flow, because i) the end-product of this disclosure will be antibiotic-free and food-grade genetic modifications; ii) the genetic elements used for engineering will not bear transposable-like sequences nor conjugational functions, which are instrumental to gene-flow in nature; and iii) the honey bee commensals are niche-specific, therefore their interaction with other microorganisms is limited. Not only can the aspects of the present disclosure limit or prevent the crossing of toxic xenobiotics through honey bee gut epithelium, but it can also remove insecticides from bee bread and decrease the honey bee metabolic burden associated with detoxification.

As described herein, the beneficial effects provided by the commensals to the host super-organism, coupled with the selective pressure caused by the presence of insecticides in the environment, can lead to a stable interaction between host and its modified commensals. Should this not be the case, the recombinant probiotics can be reapplied to colonies.

Honey bees emerge from larval stage as virtually germ-free organisms (53, 54), and their microflora is reconstituted mostly by nurse bees though trophallaxis and coprophagy (114), thus providing a plausible mechanism for the vertical transmission of recombinant probiotics (109). The present example focuses on two major classes of insecticides, OPs and SPs, and enzymes for the degradation and inactivation of these two classes of compounds (although additional aspects of the present disclosure describe degradation of other classes, for examples neonicotinoids and metabolites thereof). Moreover their capability to confer protection against insecticides has been extensively investigated as referenced hereafter. As described herein, cytochrome P450 or glutathione-S-transferase detoxifying enzymes are not exploited (44, 125, 157, 57, 133). The rationale behind this choice was to avoid any interference with the RedOx balance of the bacterial intracellular milieu, which may diminish fitness. Also, it is unclear if the P450 heme prosthetic group is present in all honey bee commensals. Instead, the emphasis as described herein will be on carboxylesterases, a class of enzymes which hydrolyze the ester bonds present in OPs and virtually all SPs. While not all carboxyesterases can hydrolyze OPs and/or SPs (125), the enzymes as described herein were selected for this capacity.

Aspects of the present disclosure has additional advantages. First, it is designed to keep the metabolic burden of the recombinant bacteria to a minimum, because enzymatic hydrolysis of esters does not require energy. Secondly, the activity of carboxylesterases can be monitored in vitro by colorimetric assays using a wide variety of substrates, thus providing a convenient screening tool for the preliminary evaluation of engineered bacteria. Thirdly, carboxylesterases do not require co-factors other than divalent metal ions (especially Zn++ and Mg++), which are naturally abundant in bee pollen and available to bacteria in the honey bee gut as well as in bee bread. This consideration is particularly relevant given that the need for expensive co-factors is a major hurdle in the field deployment of whole-cell biocatalysts. There is a compelling body of indirect and direct evidence that certain carboxylesterases can impart resistance to OPs and SPs insecticides (121, 154, 142, 125, 159, 18, 152, 47). Also, resistance to some OPs can be achieved by transforming the embryo with carboxylesterases (112, 16).

It has also been shown that the carboxylesterases have a relaxed specificity for their substrates and some carboxylesterases can even cross-hydrolyze both OP and SP (63, 158, 37, 71, 162, 168, 88). The use of promiscuous enzymes with broad-range substrate specificities is an aspect of the present disclosure because it can enhance the metabolic versatility of each engineered probiotic. In this framework, the expression of three enzymes in honey bee commensals is proposed. The first enzyme is the Methyl Parathion Hydrolase (MPH), an OP carboxylesterase, originally isolated from Plesiomonas sp. M6 (33), GenBank accession No DQ677027). This enzyme was expressed in Pseudomonas putida and it proved capable of hydrolysing a wide range of OP including methyl parathion, fenitrothion and chlorpyriphos (168). Intriguingly, MPH from Plesiomonas sp. M6 shares over 98% amino acid identity with proteins identified in several phylogenetically unrelated bacteria (71). The OP-hydrolytic activity of Pseudomonas sp.Wbc-3 carboxylesterase (which is 100% identical to MPH isolated from Plesiomonas sp. M6) was independently confirmed (117). Another major advantage of MPHs is that they appear to have a tremendous potential of improvement by in vitro mutagenesis (162, 105). The second enzyme, an SP carboxylesterase (GenBank accession No FJ688006), is encoded by the PytH gene from Sphingobium sp. JZ-1. This SP esterase was shown to be active against a panel of SPs including permethrin, deltamethrin and cypermethrin. Moreover, it did not show isomer selectivity, and it was active only on p-nitrophenyl esters of short-chain fatty acids, strongly suggesting that it does not have lipase activity (151, 168). The third enzyme is a naturally-occurring variant (Trp251Leu) of the E3 carboxylesterase (wild-type allele GenBank: AAB67728) from the blowfly Lucilia cuprina. This variant is believed to be the basis of the well documented cross-resistance between permethrin (SP) and malathion (OP) in various species (62,63, 125). Interestingly, this E3Trp251Leu variant showed a much higher activity than the wild-type enzyme for both trans permethrin (10-fold) and the more insecticidal cis permethrin (135-fold), so reducing the trans/cis preference from 27:1 to only 2:1. This mutation also shows a 1000-fold increased activity in the hydrolysis of malathion. Interestingly, the same Trp251Leu mutation was found in a malathion-resistant population of Drosophila melanogaster (63) and a closely similar variant, Trp to Gly was found in an orthologue protein present in a field population of malathion resistant hymenopterous parasitoid (166).

Modified bacteria can be fed to emerging honey bees which will be subsequently challenged with insecticides. Modified bacteria can then confer honey bees potent and potentially complete protection against orally-ingested OPs and SPs insecticides. All the insecticides according to the present disclosure can be relevant to pollinator health because they constitute the active principle in currently used insecticide preparations. Namely, the organophosphates methyl parathion, malathion, and fenitrothion; as well as the synthetic pyrethroids permethrin, deltamethrin, and cypermethrin are employed in the present disclosure.

A novel aspect of the present disclosure is that it adapts paratransgenesis from an insect pest-control strategy to a protection tool for pollinators, thus extending the concept of “probiotic” to beneficial insects. Another novel aspect is the translation to the hive stores of established technologies for the bioremediation of environmental pollutants. The strategies discussed herein are safe for many reasons. First, the engineered probiotics can be devoid of any antibiotic resistance genes. Concerns about horizontal gene transmission are misplaced because the genes that we will introduce into honey bee commensals are already widely present in the environment, especially where insecticides accumulate in the soil or water. The natural gene-flow related to insecticide-degradation pathways is substantial among these microbial populations. The proposed work will extend the metabolic capabilities, from the soil and water microorganisms into honey bees commensals. Second, as an added layer of safety, none of the genes, nor the vectors, will bear sequences related to transposable elements or to conjugational episomes, both of which facilitate horizontal gene transfer observed in nature for MPH genes (71). Third, most honey bee commensals are niche-restricted which contains interactions with other microorganisms. The proposed research is long-term and self-sustainable because it establishes a technology that is readily adaptable for problems other than those pertaining to this proposal (e.g., other pesticides and fungicides classes), and it also bears an intrinsic stability. In fact, the host may need to retain the engineered probiotics to protect against insecticides forcing the bacteria to retain the genetic modifications to preserve their environmental niche.

Approach

Embodiments of aspects of the present disclosure are disclosed below

Expression of a Pyrethroid and Organophosphate Degrading Enzymes with Broad Range Activities in Honey Bees Commensals.

As described herein, the tools for genetic manipulate of a variety of honey bee commensals have been established. In addition, a panel of honey bee commensal LABs which inhabit the mid- and hind-gut and are microaerophilic/O2-tolerant have been collected. As described herein, genetic transformations can be extended to L. kullabergensis, L melliventris, L. kimbladii, L. mellis, L. sp. AcjLac18. All these LAB are sensitive to erythromycin and kanamycin. LABs can be grown in MRS-2% fructose medium under CO₂-enriched atmosphere at all times except during harvest, wash and electroporation. Transitory exposure to regular O₂ atmosphere inhibits replication, but does not impair cell viability for at least two days. Prior to transformation, two aliquots will be inoculated in MRS-2% fructose supplemented with 2M glycine, grown until OD600=1, harvested, washed with sterile distilled H₂O, and electroporated at high-voltage, 2-2.5 Kv (93). The DNA vector can be pIB184 or a derivative thereof. This vector carries an erythromycin resistance gene, and works flawlessly in a wide variety of LABs (93, 94). As described herein, Gram-negative Gilliamella apicola have been isolated and engineered by electroporation and Snodgrassella alvi by natural transformation (preliminary data). The latter was transformed chromosomally. A suitable vector for G. apicola and S. alvi is pBBR122, an improved commercially available (MoBiTec) non-mobilizable, non-conjugational version of pBBR1MCS2 (78). This vector has a broad host range carrying different antibiotic resistance cassettes.

Expression of Insecticide-Degrading Enzymes.

Recombinant genes and genetic elements as described herein can be synthesized by GenScript (Piscathaway, N.J.). This approach can be relatively inexpensive with maximal success. The recombinant genes can be designed for host-tailored expression and to feature all necessary regulatory sequences such as promoters, and transcription termination signals. All the recombinant genes can be synthesized with a suitable promoter and termination sequence, to readily allow for subcloning into the expression vector. The constructs can be transformed into their matching hosts, and the recombinant colonies can be tested in vitro for their capability to degrade in OP and/or SP using chromogenic substrates as a model system as detailed in Objective 1.3.2. The recombinant genes encoding for 1) Mph (GenBank: DQ677027), 2) PytH (GenBank: FJ688006), and 3) the mutated version of the E3 carboxylesterase E3Trp251Leu (wild-type allele GenBank: AAB67728) can be codon-optimized according to the final expression host. From our experience as evidenced data herein, overexpression of carboxylesterases is better achieved by secreting the enzyme outside the cytosol. The signal peptides for secretion can be selected by interrogating available resources as detailed above. Each signal peptide can be extended by a few amino acids beyond the cleavage site in order to preserve the signal peptidase requirements, as defined by a Y-score, a parameter that takes into account amino acid sequences and the geometry of the surroundings, and can be customized according to the final host. While suitable promoters for all the target bacteria have been identified, tools for expression can be optimized by defining the level of expression of the in vivo transcriptome. Transcriptomic data relative to honey bee commensals retrieved from honey bee guts can be generated at the University of Florida Interdisciplinary Center for Biotechnology Research (ICBR) and analyzed in house. The level of gene expression can be used as a proxy for promoter activity. The ideal promoter(s) can have a steady, robust but not excessive activity in vivo. The newly selected promoter can be confirmed by using a fluorescent marker and then used to express the carboxylesterases. These data will allow for a precise tuning of foreign gene expression in vivo in honey bee commensals. The vectors can be matched to the host. The synthetic DNA can be excised from the GenScript vector and ligated into the final vector according to standard molecular biology techniques. Sanger sequencing can be used to confirm DNA sequences. The expression of recombinant carboxylesterases can be preliminarily detected by standard immunological methods, e.g., Western blot. To detect the recombinant proteins, polyclonal antisera against the three target carboxylesterases can be produced in rabbits. The proteins can be expressed in E. coli as MBP fusions using the pMALp2 vector (New England Biolabs). Recombinant fusion proteins can be purified using a maltose affinity-column.

Chromogenic Detection of Insecticide-Degrading Activity.

The enzymatic activity of carboxylesterases can allow for the use of chromogenic substrates as a surrogate test to measure OP or/and SP degradation by the engineered bacteria. Bacterial colonies overlaid with 100 mM 5-bromo-4-chloro-3-indolyl caprylate (X-caprylate) turn blue if carboxylesterase activity is present (158, 89). This can provide a quick method to screen bacteria. The use of this approach is demonstrated herein. In another aspect, the colonies' carboxylesterase activity can be tested using a quantitative colorimetric assay which can rely on a series of chromogenic substrates, p-nitrophenyl esters, the hydrolysis of which can be quantitated by timing the formation of p-nitrophenol at 405 nm (62, 63, 158, 89). The esterase activity of the recombinant strains can be compared to their wild-type counterparts to assess how effectively the esterase activity is expressed. An alternative method is plating bacteria on an appropriate medium containing 100 mg/ml methyl parathion or 0.4 mM cypermethrin. Both of these compounds have poor solubility producing an opaque medium. Halos of degradation can become visible around colonies capable of hydrolyzing the esters into their alcohol and acid moieties (164, 151). The cumulative data obtained herein can be used to phase out strains with sub-par performance.

Analytical Detection of Insecticide-Degrading Activity.

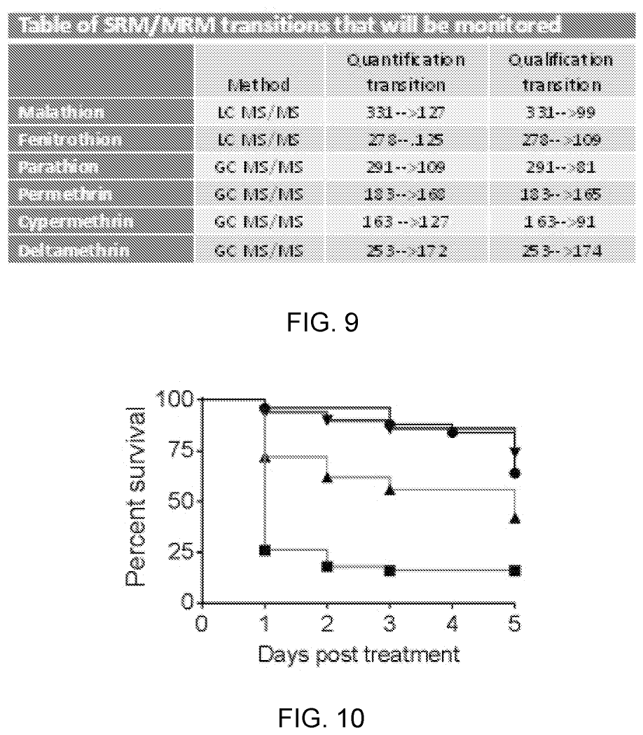

A quantitative pesticides degradation test by Liquid chromatography tandem Mass Spectrometry (LC MS/MS) and Gas Chromatography tandem Mass Spectrometry (GCMS/MS) can be performed. The strains can be grown in 50 ml liquid medium batches supplemented with 0.2 mM of the insecticides (OP: parathion, malathion, fenitrothion, and SP: permethrin, deltamethrin, cypermethrin). A 50 ml sample of the uninoculated media can be set aside as a base-line control. At days 1 post-inoculation, the bacteria can be removed from the spent media and the supernatants will be titrated. Percentages of degradation can be plotted before and after the bacterial growth. To extract the pesticides from media, citrate QuEChERS following the method of (155) can be employed. QuEChERS stands for quick, easy, cheap, efficient, rugged and safe and was first used by (11). Since then, the method has been optimized for pesticides in honey and honey bees (155, 102, 65). Standards for pyrethroids and organophosphates can be obtained from Chem Service (West Chester, Pa.). Deuterated internal standards for each class of pesticides can be obtained from Cambridge Isotope Laboratories (Andover, Mass.). For the GC MS/MS, trans-permethrin d5 can be used and for LC MS/MS, malathion d9 can be used. Pesticides can be extracted from the media supernatants using 10 ml of media and an equal volume of acetonitrile. Citrate QuEChERS salts (Agilent Technologies, anhydrous MgSO₄, NaCl, NaCitrate dehydrate, and disodium citrate sesquihydrate) and 200 μl of isotopically labeled standards at 1 μg/ml can be. The extract can be cleaned through 15 ml dispersive PSA/C18 tubes (containing anhydrous MgSO₄, PSA bonded silica and C18 bonded silica). The extract can be evaporated to 100 μl and then analyzed by LC MS/MS or GC MS/MS using methods developed by (155, 48). FIG. 9 shows an embodiment of an analysis that can be performed and the SRM/MRM ions that can be used for quantitation/qualification for each pesticide. The limit of detection and limit of quantitation can be determined by LC MS/MS on an ABI SCIEX QTRAP 6500 for malathion and fenitrothion (8) and by electron ionization (EI) GC MS/MS on an Agilent 7890/7000C GC MS/MS instrument for parathion, permethrin, cypermethrin and deltamethrin (Weist et al). All methods will be optimized for sensitivity. FIG. 9 lists the SRM/MRM transitions that can be used for quantitation and for qualifications. The results of the biodegradation test can be a relevant factor for the selection and every strain with poor performance will be phased out. Only one enzyme per insecticide class will be followed up in each strain. In any given strain, if the E3Trp251Leu, which has dual-activity on both OP and SP, outperforms the other enzymes, Lactic Acid Bacteria showing protective activity could probably be developed into GEM, Gram-positive enhanced matrix (21) which may open interesting future applications of the technology proposed herein.

Identification of the Best Protecting Probiotics.

### Whole-Cell Bioremediation of the Food Sources.

From a biotechnological perspective, the decontamination of honey bee food-sources can be assimilated into a bioremediation process to remove pollutants from the environment. Such an approach can adapt knowledge already gained from the bioremediation of contaminated soils and sludge. Data as shown herein demonstrate such, and the whole-cell biocatalyst strategy has proven extremely effective for a number of cases (179, 180, 181). Six insecticides will be alternatively tested (organophosphates methyl parathion, malathion, fenitrothion; and the synthetic pyrethroids permethrin, deltamethrin, cypermethrin).

At this point, the bacterial strains to be used can depend on the results above. Once completed, different strain-promoter combinations can offer a range of protection. Minimally, F. fructosus and L. kunkeei, each one expressing the three selected recombinant genes, can be used to ferment simulated bee bread (10% pollen+20% fructose). Other fructophilic bacteria have been collected, which may also be useful in the decontamination process. Their deployment can be evaluated.

The challenges can be delivered in simulated bee bread. The amount of simulated bee bread used daily by each honey bee is approximately 25 ml, therefore the dilutions of each insecticide in acetone can be calculated so that a 25 ml aliquot delivers the desired xLC₅₀, daily. The insecticide-spiked bee bread can then be inoculated with the native bacteria, as a negative control, or with their engineered counterpart. The difference in survival can then be measured and statistically analyzed.

Capped combs containing emerging adult honey bees can be obtained from several colonies at the University of Florida apiary (Gainesville, Fla., USA). Combs with emerging bees can be placed in an incubator under simulated hive conditions (33° C., 65% RH) to allow bees to emerge. Twenty-four hours later, newly emerged bees, which cannot fly and have minimal stinging capability, can be brushed from the frames into holding tubs and allowed to intermix to minimize variation in the experimental population. The bees then can be distributed into clean cages (10 or 20 bees/cage), returned to the incubator and fasted for three hours.

Two doses of bacteria, (106 and 104 CFUs ml-1) can be mixed with simulated pollen, which can be fed to honey bees. Survival rates between the control and the treatment can be measured. Oral LC₅₀ values for each insecticide can be extrapolated from (182, 187) or determined in house.

Based upon data as described herein, honey bees can be challenged daily with 10xLC₅₀ of insecticide, and can be scaled-up or -down as needed. Each insecticide can be tested individually. The Kaplan-Meier method can be applied to obtain the survival fractions following pesticide challenge in honey bees. Using the Mantel-Haenszel log rank test, the P-value for statistical differences between surviving pesticide challenges among treated groups compared to those dosed with the corresponding strains lacking the pesticide-degrading enzymes can be discerned at the 95% confidence interval. The strains in appropriate combinations can also be tested for their ability to protect against more than one insecticide and belonging to more than one class. Data suggest that relatively low numbers of honey bees may be needed to test the efficacy of the treatments. In fact, 50 honey bees in the treatment and control groups can yield desired results. The capability to incorporate hundreds of bees daily can be utilized in experiments even up to 9-10 months a year.

Cell-Free Enzymatic Bioremediation of the Food Sources.

The use of formulated enzymes rather than whole cell biocatalysts is a process known as enzymatic bioremediation, the feasibility of which has been shown in different settings (reviewed in 174, 185) and references therein). As described herein, bee pollen can be a suitable target for this kind of intervention as an alternative or complement to whole cell catalysis described above. As shown herein, the recombinant carboxylesterase, when expressed in E. coli and purified, can reduce the toxicity of 40xLC50 cypermethrin to honey bees when tested in a 20% fructose syrup model system (FIGS. 8A-8C). To further this approach all three carboxylesterases identified in the above section can be expressed and isolated. Optimal concentrations and enzyme-to-insecticide ratios can be identified in the same fructose syrup model. Subsequently, fresh bee pollen, which is 20-35% water, thus perfectly adequate for enzymatic bioremediation (182), can be spiked with insecticide (OP or SP) by spraying. A portion of the spiked bee pollen can serve as a control, whereas another can be enzymatically bioremediated by spraying a solution of the recombinant protein. Bees can be allowed to feed, and the survival of treatment VS control can be measured.

Symbiont-Mediated Bioremediation.

A maximum of 16 species/gene combination (6 LAB, G. apicola and S. alvi), each one expressing an OP recombinant gene, a SP recombinant gene or, the synthetic/purified E3 Trp251Leu carboxylesterase, which can degrade both OP and SP, can be tested. The LC₅₀ delivered in 25 ml of feed for each insecticide can serve as a benchmark to test the efficacy of each recombinant commensal which can be fed to emerging honey bees at 105 CFUs ml-1 in 125 g/I of sucrose syrup. This dose is derived from available data showing that probiotics administered at 104-105 CFUs ml-1 in feed have a remarkable benefit (15, 30, 150,171, 172, 173, 181). Data demonstrating the beneficial effects of selected commensals for honey bees can allow dosing the syrup with the recombinant strains, and compare results obtained with syrup containing the isogenic, negative control strains lacking the enzymatic activity. Bacteria can be grown according to their required selective media and antibiotic, and then can be added to the syrup at the appropriate concentrations when at mid-to-late log phase is reached in order to preserve their metabolic activity. Next, the capacity of each strain can be tested for conferring protection against 1xLC₅₀ of the cognate insecticide. It is worth at this point to recapitulate that eight different strains, each carrying either two (possibly, 1) recombinant genes (1 OP degrading, 1 SP degrading or, alternative, the E3Trp251Leu only) can be assessed in this bioassay. At this stage, only four insecticides (2 OP methyl parathion, malathion; and 2 SP permethrin, deltamethrin) can be evaluated singularly. Strains expressing Methyl Parathion Hydrolase (MPH) can be tested against OPs, and the strains expressing PytH can be tested against SPs. Strains expressing the naturally-occurring variant E3 (Trp²⁵¹Leu) can be tested against combinations of OPs and SPs. For controls, insecticide-laced bee bread can contain equal CFUs of the naked vector strains. The mortality of bees fed protective (modified) commensals can be plotted against the mortality of bees fed bee bread containing vector controls, and the results can be evaluated over the course of 10 days. Survival rates can be determined using Kaplan-Meier test. All the following numbers may need +/− adjustments depending on the differences between the treatments and the controls. This approach can entail 64 batches (8 strains×1-2 genes×for 4 insecticides) of 3×20 honey bees each, total 3480 honey bees. The 8 control batches (each control strain carrying the naked DNA vector) on 4 insecticides will add 2000 honey bees, totaling 5480 bees. Should robust protection against 1xLC₅₀ be achieved, then subsequent challenges can be performed using higher challenge doses, e.g., 5xLC₅₀-20xLC₅₀. It may not be necessary to test doses >20xLC50 since these levels of exposure would be expected to be rare catastrophic events and thus not worth addressing. In contrast to previous test, the challenge against 5-20xLC₅₀ can be performed against four insecticides (2 OPs+2 SPs) mixed together. This approach can be performed by mixing together 106 CFUs ml-1 of bee bread and each of the strains which conferred protection against at least 10xLC₅₀. The test can have six replica (6×30 bees each). The controls can be the same strains carrying the naked vector. If protection is achieved, the experiment can be repeated with 1/10 of CFUs.

Engineering Antibiotic Resistance-Free, Food-Grade Genetically Modified Commensals that Stably Express the Enzymes Needed for Pesticide Catabolism and Confer Protection for Pesticide-Exposed Honey Bees.

The generation of this kind of strains is an established, two-step process which is summarized in (55, 79, 108). As described herein, this strategy is adapted by inserting the carboxylesterase(s) and the m-Cherry (optionally) as a bi-cistronic minioperon.

### Chromosomal Transformation.

At this stage, there can be around 5-7 strains. The suicide vector pCVD442 and its permissive host E. coli strain S17-1, which have an over 20 years record of successful chromosomal insertions in a very broad range of hosts (40, 111, 170), can be used in this project. The overall scheme of the recombinant suicide vectors is shown in (FIGS. 3A and 3B). All the components can be customized to the final host, e.g. the promoter, the codon usage for the open reading frames (ORFs), Shine-Dalgarno, the terminator and the target sequences. Insertion sites can be selected utilizing the information gained as described above. Rare-cutter restriction enzymes (i.e. NotI, PmeI, SwaI) can be included outside the target sequences to allow modular excision of the fragment from the synthetic construct prior to cloning into the suicide vector. Recombinant suicide vectors can be introduced into their target host by electroporation as detailed above. The first step, chromosomal integration, can be tested by selection on plates containing the antibiotic and double checked, by sequencing the regions flanking the inserted site. One or more positive colonies can be grown in liquid medium in absence of antibiotic selection for about 100 generations. At the end of this growth, single colonies can be replica-plated with or without selection. The colonies sensitive to the antibiotic can be tested for GFP expression by shining a portable UV light in a dark room and confirmed by a cell sorter, for example a Sony SH800. Furthermore they can be confirmed for insecticide degradation by reiterating the procedure described above. The strains showing degrading activity can then be tested for honey bee protection as described above.

### Testing Vertical Transmission.

Plasm id-engineered bacteria can be used to test if the engineered microbiota can be passed down to newly emerging honey bees. Optional fluorescent markers (for example, GFP or m-Cherry) can be used for tracking the bacterial flow between honey bees and for checking the stability of the genetic modifications over time in vivo. A procedure as described above can be adapted for this purpose. Color-marked honey bees reconstituted with recombinant microflora, and their fecal matter, can be intermixed with unmarked newly emerged honey bees not having the recombinant microflora, thus allowing for the determination of if those with the recombinant microflora trophallactically pass the microflora to other bees. Nurse bees containing the recombinant microflora can be color-labelled to distinguish them from emerging honey bees not having the microflora. The presence of recombinant bacteria in the newly emerged honey bees can be detected by flow-cytometry analysis. Should it be determined that the recombinant microflora can be vertically transmitted, depending on the resources and time available at the time, experiments can be repeated with insecticide-degrading plasmid-engineered microbiota and the newly emerged honey bees can be challenged with 1xLC₅₀ of the suitable insecticide.

### Example 2

Honey bees (Apis mellifera) are key pollinators for a vast array of crops, with a global added value in the tens of billions of dollars for the US economy alone. One of the major stressors leading to the current honey bee colony losses is the sub-lethal exposure to pesticides. To address this problem, compositions and methods are described herein to mitigate and/or overcome insecticide poisoning by exploiting a novel application of paratransgenesis. As described herein, hive-related bacteria, engineered to degrade OPs and SPs insecticides efficiently, can confer potent protection against insecticide poisoning, thus complementing and synergizing with the honey bees' own defensive mechanisms. Pollen contaminated with insecticides can be assimilated to a sludge in need of bioremediation, thus allowing the potential to tap into strategies and technologies developed over decades of environmental biotechnology. This green technology is sometimes referred to as “bioaugmentation”. More specifically, as described herein, genetically engineering bee bread-adapted bacteria, as well as honey bee symbionts sensu stricto, enabling them to degrade two classes of insecticides, organophosphates (OP) and synthetic pyrethroids (SP). Compositions, methods, and strategies as described herein can lead to a safer environment for honey bees. This approach to bioremediation is referred to as “whole cell biocatalysis,” whereby the enzymatic reaction is catalyzed/operated by cells as opposed to purified enzymes. Furthermore, the purified carboxylestrases, recombinantly produced and appropriately applied to the hive food stores, can enhance the protection. This latter approach to bioremediation is referred to as “enzymatic bioremediation”.

Methods and strategies as described herein can entail the genetic manipulation of honey bee commensals to degrade OP and SP insecticides using insecticide-degrading carboxylesterases derived from soil and water-borne bacteria (34, 71, 139). Last, bacteria isolated from fresh bee bread can be molecularly bred to efficiently degrade insecticides adding an even deeper layer of protection. The long-term goal of this proposal is to protect honey bees against OP and SP insecticides.

### The Proposal.

Compositions and methods as described herein can utilize four materials:

1) The honey bee Lactic Acid Bacteria (LAB) engineered to express the carboxylesterase.

2) An E. coli strain engineered to express the carboxylesterase.

3) The purified carboxylesterase.

4) A consortium of bacteria derived from bee bread, subjected to in vitro evolution by genome shuffling and selected for over one year for being capable of using various insecticides as a sole carbon source. In actuality, 4 consortia are described herein, each one capable of growing on a different insecticide (the neonicotinoid, imidacloprid; the neonicotinoid, clothianidin; the synthetic pyrethroid, cypermethrin; and the orhanophosphate, methylparathion) as a sole carbon source.

The presented data were generated by challenging honey bees with the synthetic pyrethroid cypermethrin (which is highly toxic to honey bees), bioremediated with different combinations of the bacteria described above.

Whole-Cell Remediation:

FIGS. 7A-7B are graphs illustrating data from a 40xLC₅₀ cypermethrin challenge. Simulated bee bread was spiked with the highly toxic cypermethrin, fermented with a mix of bacteria and fed to the honey bees. This mixture of bacteria contains carboxylesterase-producing LAB, carboxylesterase producing E. coli, and bacteria form the consortium selected on cypermethrin (see below). Cypermethrin concentration was adjusted to deliver 40xLC₅₀ daily (assuming that each honey bee would take 25 ml, daily). FIG. 7A shows survival data. FIG. 7B shows the time to death. Cypermethrin-spiked medium was not withdrawn at any time.

Enzymatic Biomediation:

FIGS. 8B-8C are graphs illustrating data from a 40xLC₅₀ cypermethrin challenge. Cypermethrin challenge delivered by fructose syrup. 20% fructose syrup was spiked with cypermethrin and bioremediated with the purified carboxylesterase. Cypermethrin concentration was adjusted to deliver 40xLD₅₀ daily (assuming that each bee would take 25 ml of the syrup daily). 10×5 bees were used in each batch. Cypermethrin-spiked syrup was not withdrawn at any time. Media consumption shows that bees kept feeding during the experiment. FIG. 8B illustrates a survival curve of bees without protein (−) (bottom; circles), with protein (+) (middle; squares), and control (top; triangles). FIG. 8C shows the amount of medium consumed without protein (−) and with protein (+).

FIG. 10 is a survival curve of a 40xLD50 cypermethrin challenge delivered by fructose syrup as in the experiment of FIGS. 8B-8C. Black line (very top line with circles). Fructose syrup only. Red line (bottom line with squares). Fructose syrup+cypermethrin Green line (middle line with triangles). Fructose syrup+cypermethrin+protein. Blue line (second to the top line with upside down triangles). Fructose syrup made with AB-mineral medium supernatant of the consortium, spiked with cypermethrin at the beginning of the growth.

Conclusions

A) The data provide strong support to the overall strategy. There remains considerable debate about what concentration of insecticide should be considered “field concentration.” It is estimated that the challenge delivered herein in an amount of cypermethrin that is logarithmically (at least 10-fold, but likely, 100- to 1000-fold) higher than the average exposure in the field. It is reasonable to surmise that, the honey bees can be conferred with strong protection against any reasonable intoxication that they may be subjected to in the field according to compositions and methods as described herein.

B) The experimental approach involving the simulated bee pollen bioremediated with “whole cell biocatalysts”, (whereby the chemical reaction is catalyzed by whole cells) is feasible. Further embodiments of this approach can be undertaken, including genetic manipulations for enzyme secretion (to facilitate improved contact between the enzymes and the insecticides), and the selection of engineered bacteria.

C) Suitable results were also obtained when the challenge was delivered via a homogeneous fructose solution (syrup). This medium can easily be fed to honey bees, which use their ligula (tongue) to neatly retrieve it from the feeding device without causing spillage into the cups nor onto their own heads. As a result, the difference between the treatments and the controls can more readily be detectable and reproducible (FIGS. 8B-8C, FIG. 10). Moreover, the bioremediation with the purified enzyme, devoid of any component of the cellular milieu, provides evidence that the presence of the enzyme is instrumental for detoxification.

D) The bacterial consortium selected on cypermethrin as a sole carbon source (described hereafter with detail) is effective in bioremediation (FIG. 10, blue line with upside down triangles). In this respect, both the entire consortia as a whole and, possibly, the individual species can provide the metabolic capability to metabolize the insecticides in the food stores. Although some of these species may be pathogenic to honey bees, the natural fermentation process virtually sterilizes bee bread, thus ultimately abolishing their pathogenic effect (Pain, J., Maugenet, J. (1966) Recherches biochimiques et physiologiques sur le pollen emmagasine' par les abeilles. Ann, Abeille 9, 209-236. Confirmed by data herein—see FIG. 12). In this respect, another intervention strategy can involve the delivery of bacterial consortium or some of its species to fresh bee bread, with the understanding that these bacteria will be cleared during the fermentation process.

E) An aspect of this proposal resides in considering the hive stores as a substrate for whole cell biocatalysis and enzymatic bioremediation, which was defined above. This approach can enable the removal of pollutants from the environment. This enzymatic bioremediation can provide support for the overall idea that carboxylesterases can bioremediate food stores as a result of the enzyme's remarkable capacity for decontamination. On the other hand, the whole cell biocatalysis platform may provide a less labor-intensive, less expensive, and more sustainable protection in that engineered wild-type bacteria can self-support once they have been introduced in the system.

### Molecular Breeding and Selection of Consortia

Described herein is an approach that can generate microbial communities and/or single microbial species that, while being adapted to the hive food stores, can degrade, besides the insecticides described above, recalcitrant insecticides including, but not limited to, neonocotinoids. Compositions, methods, and strategies as described herein can enable novel applications of the knowledge developed for the removal of pollutants from the environment. As described herein, pollen/bee bread-adapted bacteria can remove the same compounds from the honey bee food stores.

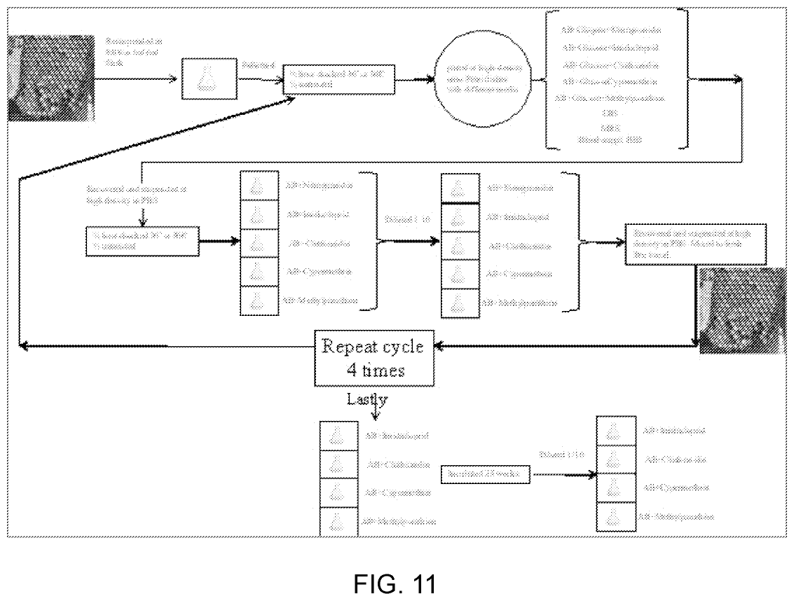

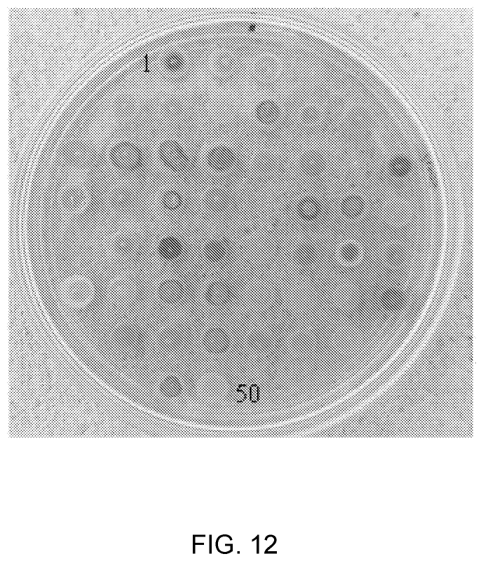

As described in the present example, native bacteria can be employed, naturally present in bee bread. From freshly isolated bee bread collected at the University of Florida apiary, bacteria can be grown on, alternatively, rich media and mineral media where the sole carbon source was the insecticide. Nitroguanidin was also used because that this compound may improve the capability of bacteria to use the nitrogen present within the neonicotinoid molecules as a nitrogen source. Incidentally, attempts to use the insecticides as a sole nitrogen source (instead of carbon) failed because diazotrophs were selected, bacteria that can fix atmospheric nitrogen. The bacteria underwent cycles of bentonic and planctonic growth to foster the exchange genetic materials amongst the pan-genome, while minimizing the selection of bacterial mats, where the degradation is operated by groups of bacterial species living in tight contact mutually assisting in the degradation process. The overall scheme of the selection process is presented in (FIG. 11).

FIG. 11 is a diagram illustrating an embodiment of a molecular breeding procedure according to the present disclosure. Over the course of one year, bacteria, which can be derived from ripening bee bread, can be cycled through bentonic growth on rich media or AB mineral media and planctonic growth on AB mineral media. AB mineral media can contain the 4 insecticides (imidacloprid, clothianidin, cypermethrin, and methylparathion) as the sole carbon source. After four cycles, bacteria can be transferred in planctonic AB minimal media and subcultured for about six months. At the end of this period, the surviving bacteria can be plated on rich media, selected for morphological differences and further characterized.

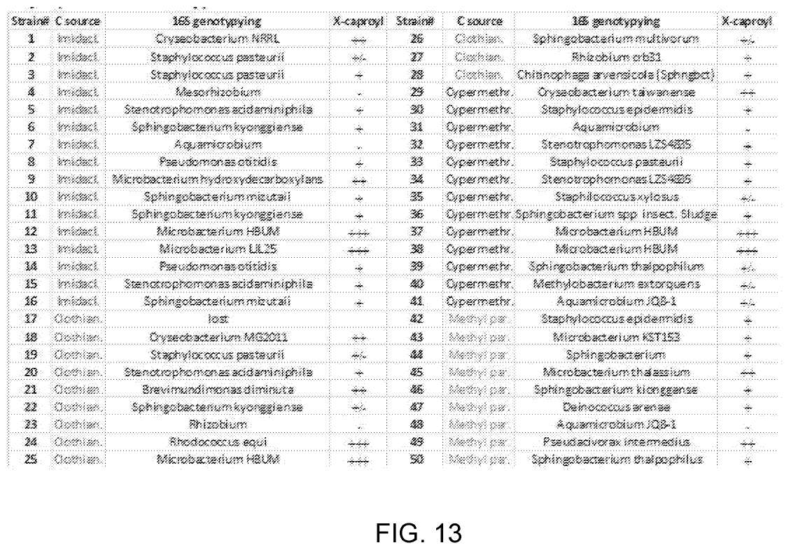

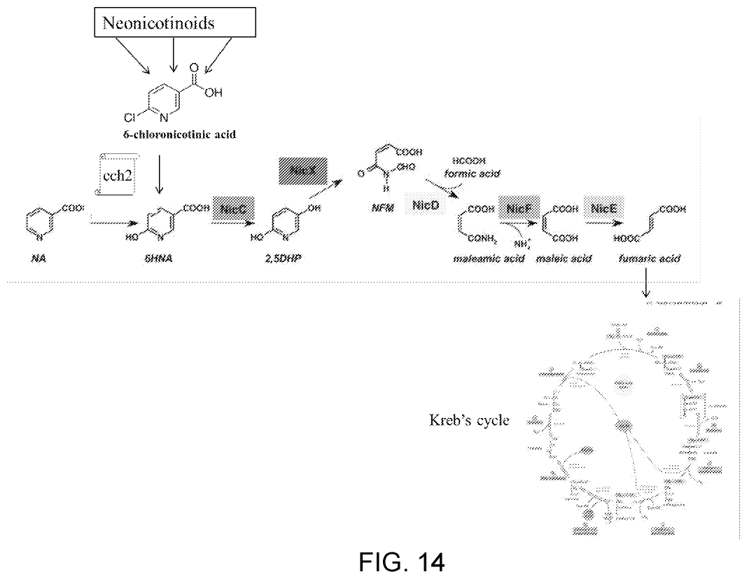

The consortia have partially been characterized by plating the bacteria to single colony on different media. The consortia as described herein in certain aspects may be composed also by species that cannot be cultured. The rationale for focusing on using culturable bacteria is that from a biotechnological perspective, bacteria that cannot be grown to a pure culture have a reduced practical utility. Colonies with different morphology were selected, and further analyzed. The carboxylesterase activity of 50 clones has been crudely assessed by overlaying X-caproylate (see Example 1 above and FIG. 54). The presence of carboxylesterase(s) is encouraging because a major step in the deactivation of synthetic pyrethroids and organophosphate insecticides (but not nicotinoids) is the dissolution of their ester bonds. The same 50 clones were genotyped via 16S DNA, and many of them have close relatives in the databases which are known for their insecticide-degrading capabilities, thus validating our overall strategy (FIG. 13).

FIG. 12 The 50 pure culture bacteria described in the following FIG. 13 were spotted on a BHI (Brain heart infusion) Petri dish and overlaid with X-caproyl, a chromogenic substrate which detects carboxylesterase activities by turning blue. The activity was arbitrarily assigned to 5 groups: +++ robust blue color within 5 minutes; ++ robust blue color, + weak blue color within 1 hour; +/− very pale color, marginally detectable at one hour, and − no blue color detectable.

FIG. 13 is a table of clones of FIG. 11 showing results of 16S DNA genotyping of 50 single-colony clones isolated from a molecular breeding experiment. Using the specific AB-insecticides medium, colonies were isolated and marked below in color. Genomic DNA was isolated from each colony, and amplified with universal primers. The resulting DNA bands were Sanger-sequenced, and sequences were matched against existing data banks. The table shows the best match of the clones isolated from bee bread; however in most cases, the match was not 100%, suggesting that these isolates may represent novel subtypes.

### REFERENCES FOR EXAMPLE 1 AND 2

(1) https://www.ars.usda.gov/oc/br/ccd/index/(2)
(2) http://usda.mannlib.cornell.edu/usda/current/CostPoll/CostPoll-12-22-2016.pdf
(3) http://www.usda.gov/documents/ReportHoneyBeeHealth.pdf
(4) http://www.europarl.europa.eu/sides/getDoc.do?type=REPORT&reference=A7-2011-0359&language=EN
(5) http://www.cost.eu/COST_Actions/fa/FA0803
(6) http://articles.extension.org/pages/58013/honey-bee-winter-loss-survey
(7) Alberoni D, Gaggia F, Baffoni L, Di Gioia D. Beneficial microorganisms for honey bees: problems and progresses. Appl Microbiol Biotechnol. 2016 November; 100(22):9469-9482. Epub 2016 Oct. 8.
(8) Alder L, Greulich K, Kempe G, Vieth B. Residue analysis of 500 high priority pesticides: better by GC-MS or LC-MS/MS? Mass spectrometry reviews. 2006 Nov. 1; 25(6):838-65.
(9) Alippi A M, Reynaldi F J. Inhibition of the growth of Paenibacillus larvae, the causal agent of American foulbrood of honeybees, by selected strains of aerobic spore-forming bacteria isolated from apiarian sources. J Invertebr Pathol. 2006 March; 91(3): 141-6.
(10) Aljedani D M, Almehmadi R M. Effects of some insecticides on longevity of the foragers honey bee worker of local honey bee race Apis mellifera jemenatica. Electron Physician. 2016 Jan. 15; 8(1):1843-9.
(11) Anastassiades, M., Lehotay, S. J., Stajnbaher, D., and Schenck, F. J., Fast and easy multiresidue method employing acetonitrile extraction/partitioning and “dispersive solid-phase extraction” for the determination of pesticide residues in produce. Journal of Aoac International, 86(2): 412-431 (2003).
(12) Anderson, K. E., Sheehan, T. H., Mott, B. M., Maes, P., Snyder, L., Schwan, M. R., Walton, A., Jones, B. M., Corby-Harris, V. (2013) Microbial ecology of the hive and pollination landscape: bacterial associates from floral nectar, the alimentary tract and stored food of honey bees (Apis mellifera). PLoS One. 8:e83125.
(13) Anderson K E, Carroll M J, Sheehan T, Lanan M C, Mott B M, Maes P, Corby-Harris V. Hive-stored pollen of honey bees: many lines of evidence are consistent with pollen preservation, not nutrient conversion. Mol Ecol. 2014 December; 23(23):5904-17.
(14) Audisio M C, Terzolo H R, Apella M C. Bacteriocin from honeybee beebread Enterococcus avium, active against Listeria monocytogenes. Appl Environ Microbiol. 2005 June; 71(6):3373-5.
(15) Audisio M C. Gram-Positive Bacteria with Probiotic Potential for the Apis mellifera L. Honey Bee: The Experience in the Northwest of Argentina. Probiotics Antimicrob Proteins. 2017 March; 9(1):22-31.
(16) Benedict M Q, Scott J A, Cockburn A F. High-level expression of the bacterial opd gene in Drosophila melanogaster: improved inducible insecticide resistance. Insect Mol Biol. 1994 November; 3(4):247-52.
(17) Berasategui A, Shukla S, Salem H, Kaltenpoth M. Potential applications of insect symbionts in biotechnology. Appl Microbiol Biotechnol. 2016 February; 100(4):1567-77.
(18) Birner-Gruenberger R, Bickmeyer I, Lange J, Hehlert P, Hermetter A, Kollroser M, Rechberger G N, Kühnlein R P. Functional fat body proteomics and gene targeting reveal in vivo functions of Drosophila melanogaster α-Esterase-7. Insect Biochem Mol Biol. 2012 March; 42(3):220-9.
(19) Biswas, I., Jha, J. K., Fromm, N. (2008) Shuttle expression plasmids for genetic studies in Streptococcus mutans. Microbiology 154, 2275-82.
(20) Bloch, G. J. (2010) The social clock of the honeybee. Biol Rhythms 25, 307-17.
(21) Bosma T, Kanninga R, Neef J, Audouy S A, van Roosmalen M L, Steen A, Buist G, Kok J, Kuipers O P, Robillard G, Leenhouts K. Novel surface display system for proteins on non-genetically modified gram-positive bacteria. Appl Environ Microbiol. 2006 January; 72(1):880-9.
(22) Buffie C G, Bucci V, Stein R R, McKenney P T, Ling L, Gobourne A, No D, Liu H, Kinnebrew M, Viale A, Littmann E, van den Brink M R, Jenq R R, Taur Y, Sander C, Cross J R, Toussaint N C, Xavier J B, Pamer E G. Precision microbiome reconstitution restores bile acid mediated resistance to Clostridium difficile. Nature. 2015 Jan. 8; 517(7533):205-8.
(23) Calderone, N. W. (2012) Insect pollinated crops, insect pollinators and US agriculture: trend analysis of aggregate data for the period 1992-2009. PLoS One. 2012; 7(5).
(24) Campbell J W, Cabrera A R, Stanley-Stahr C, Ellis J D. An Evaluation of the Honey Bee (Hymenoptera: Apidae) Safety Profile of a New Systemic Insecticide, Flupyradifurone, Under Field Conditions in Florida. J Econ Entomol. 2016 October; 109(5):1967-72.
(25) Cheng D, Guo Z, Riegler M, Xi Z, Liang G, Xu Y. Gut symbiont enhances insecticide resistance in a significant pest, the oriental fruit fly Bactrocera dorsalis (Hendel). Microbiome. 2017 Feb. 1; 5(1):13.
(26) Christen V, Fent K. Exposure of honey bees (Apis mellifera) to different classes of insecticides exhibit distinct molecular effect patterns at concentrations that mimic environmental contamination. Environ Pollut. 2017 July; 226:48-59.
(27) Coppin C W, Jackson C J, Sutherland T, Hart P J, Devonshire A L, Russell R J, Oakeshott J G. Testing the evolvability of an insect carboxylesterase for the detoxification of synthetic pyrethroid insecticides. Insect Biochem Mol Biol. 2012 May; 42(5):343-52
(28) Corby-Harris, V., Maes, P., Anderson, K. E. (2014) The Bacterial Communities Associated with Honey Bee (Apis mellifera) Foragers. PLoS One. 9:e95056.
(29) Corby-Harris V, Snyder L A, Schwan M R, Maes P, McFrederick Q S, Anderson K E. Origin and effect of Alpha 2.2 Acetobacteraceae in honey bee larvae and description of Parasaccharibacter apium gen. nov., sp. nov. Appl Environ Microbiol. 2014 December; 80(24):7460-72.
(30) Corby-Harris V, Snyder L, Meador C A, Naldo R, Mott B, Anderson K E. Parasaccharibacter apium, gen. nov., sp. nov., Improves Honey Bee (Hymenoptera: Apidae) Resistance to Nosema. J Econ Entomol. 2016 April; 109(2):537-43.
(31) Cornman, R. S., Tarpy, D. R., Chen, Y., Jeffreys, L., Lopez, D., Pettis, J. S., vanEngelsdorp, D., Evans, J. D. (2012) Pathogen webs in collapsing honey bee colonies. PLoS One 7:e43562.
(32) Coutinho-Abreu, I. V., Zhu, K. Y., Ramalho-Ortigao, M. (2010) Transgenesis and paratransgenesis to control insect-borne diseases: current status and future challenges. Parasitol Int 59, 1-8.
(33) Cui Z, Li S, Fu G. Isolation of Methyl Parathion-Degrading Strain M6 and Cloning of the Methyl Parathion Hydrolase Gene Appl. Environ. Microbiol. October 2001 67:4922-4925;
(34) Cycoń M, Piotrowska-Seget Z. Pyrethroid-Degrading Microorganisms and Their Potential for the Bioremediation of Contaminated Soils: Front Microbiol. 2016 Sep. 15; 7:1463. eCollection 2016.
(35) Dai, P., Jia, H., Jack, C., Geng, L., Liu, F., Hou, C., Ellis, J. D. 2016. Bt CryThe toxin does not impact the survival and pollen consumption of Chinese honey bees, Apis cerana cerana (Hymenoptera, Apidae). Journal of Economic Entomology 109(6): 2259-2263.
(36) Dai P, Jack C J, Mortensen A N, Ellis J D. Acute toxicity of five pesticides to Apis mellifera larvae reared in vitro. 2017. Pest Manag Sci.
(37) Daumann L J, McCarthy B Y, Hadler K S, Murray T P, Gahan L R, Larrabee J A, 011 is DL, Schenk G. Promiscuity comes at a price: catalytic versatility vs efficiency in different metal ion derivatives of the potential bioremediator GpdQ. Biochim Biophys Acta. 2013 January; 1834(1):425-32.
(38) Desneux N, Decourtye A, Delpuech J M. The sublethal effects of pesticides on beneficial arthropods. Annu Rev Entomol. 2007; 52:81-106.
(39) Dewar A M. The adverse impact of the neonicotinoid seed treatment ban on crop protection in oilseed rape in the United Kingdom. Pest Manag Sci. 2016 Dec. 26. doi: 10.1002/ps.4511. [Epub ahead of print].
(40) Donnenberg M S, Kaper J B. Construction of an eae deletion mutant of enteropathogenic Escherichia coli by using a positive-selection suicide vector. Infect Immun. 1991 December; 59(12):4310-7.
(41) Douglas G L, Klaenhammer T R. Directed chromosomal integration and expression of the reporter gene gusA3 in Lactobacillus acidophilus NCFM. Appl Environ Microbiol. 2011 October; 77(20): 7365-71.
(42) du Rand E E, Smit S, Beukes M, Apostolides Z, Pirk C W, Nicolson S W. Detoxification mechanisms of honey bees (Apis mellifera) resulting in tolerance of dietary nicotine. Sci Rep. 2015 Jul. 2; 5:11779.
(43) Durvasula, R. V., Gumbs, A., Panackal, A., Kruglov, O., Aksoy, S., Merrifield, R. B., Richards, F. F., Beard, C. B. (1997) Prevention of insect-borne disease: an approach using transgenic symbiotic bacteria. Proc Natl Acad Sci USA 94, 3274-8.
(44) Eaton D L1. Biotransformation enzyme polymorphism and pesticide susceptibility. Neurotoxicology. 2000 February-April; 21(1-2): 101-11.
(45) Engel P, Stepanauskas R, Moran N A. Hidden diversity in honey bee gut symbionts detected by single-cell genomics. PLoS Genet. 2014 Sep. 11; 10(9):e1004596.
(46) Fang, W., Vega-Rodriguez, J., Ghosh, A. K., Jacobs-Lorena, M., Kang, A., St Leger, R. J. (2011) Development of transgenic fungi that kill human malaria parasites in mosquitoes. Science. 331, 1074-7.
(47) Faucon F, Gaude T, Dusfour I, Navratil V, Corbel V, Juntarajumnong W, Girod R, Poupardin R, Boyer F, Reynaud S, David J P. In the hunt for genomic markers of metabolic resistance to pyrethroids in the mosquito Aedes aegypti: An integrated next-generation sequencing approach. PLoS Negl Trop Dis. 2017 Apr. 5; 11(4):e0005526.
(48) Feo, M. L., Eljarrat, E., and Barcelo, D., Performance of gas chromatography/tandem mass spectrometry in the analysis of pyrethroid insecticides in environmental and food samples. Rapid Commun Mass Spectrom, 25(7): 869-76 (2011).
(49) Forsgren, E., Olofsson, T. C., Vásquez, A., Fries, I. (2010) Novel lactic acid bacteria inhibiting Paenibacillus larvae in honey bee larvae Apidologie. 41, 99-108.
(50) Gallai, N., Salles, J-M., Settele, J., Vaissiére, B. E. (2009) Economic valuation of the vulnerability of world agriculture confronted with pollinator decline. Ecological Economics 68, 810-821.
(51) Gill R J, Ramos-Rodriguez O, Raine N E. Combined pesticide exposure severely affects individual and colony-level traits in bees. Nature. 2012 Nov. 1; 491(7422):105-8.
(52) Gilliam, M. (1979). Microbiology of pollen and bee bread: the genus Bacillus. Apidologie 10, 269-274.
(53) Gilliam M, Prest D B. Microbiology of feces of the larval honey-bee, Apis-mellifera. J lnvertebr Pathol. 1987; 49: 70-75.
(54) Gilliam M. 1971. Microbial sterility of the intestinal content of the immature honey bee, Apis mellifera. Ann. Entomol. Soc. Am. 63:315-316.
(55) Goh Y J, Azcárate-Peril M A, O'Flaherty S, Durmaz E, Valence F, Jardin J, Lortal S, Klaenhammer T R. Development and application of a upp-based counterselective gene replacement system for the study of the S-layer protein SIpX of Lactobacillus acidophilus NCFM. Appl Environ Microbiol. 2009 May; 75(10):3093-105.
(56) Gómez-Gallego C, Junnila J, Männikkö S, Hämeenoja P, Valtonen E, Salminen S, Beasley S. A canine-specific probiotic product in treating acute or intermittent diarrhea in dogs: A double-blind placebo-controlled efficacy study. Vet Microbiol. 2016 Dec. 25; 197:122-128.
(57) Gong Y, Diao Q. Current knowledge of detoxification mechanisms of xenobiotic in honey bees. Ecotoxicology. 2017 January; 26(1):1-12.
(58) Goulson, D., Nicholls, E., Botias, C., Rotheray, E. L. (2015) Bee declines driven by combined stress from parasites, pesticides, and lack of flowers. Science. 347, 1255957.
(59) Gregorc, A., Ellis, J. D. 2011. Cell death localization in situ in laboratory reared honey bee (Apis mellifera L.) larvae treated with pesticides. Pesticide Biochemistry and Physiology 99: 200-207.
(60) Gregorc, A., Evans, J. D., Scharf, M., Ellis, J. D. 2012. Gene expression in honey bee (Apis mellifera) larvae exposed to pesticides and Varroa mites (Varroa destructor). Journal of Insect Physiology 58: 1042-1049.
(61) Gupta S, Allen-Vercoe E, Petrof E O. Fecal microbiota transplantation: in perspective. Therap Adv Gastroenterol. 2016 March; 9(2):229-39.
(62) Heidari R, Devonshire A L, Campbell B E, Bell K L, Dorrian S J, Oakeshott J G, Russell R J. Hydrolysis of organophosphorus insecticides by in vitro modified carboxylesterase E3 from Lucilia cuprina. Insect Biochem Mol Biol. 2004 April; 34(4):353-63.
(63) Heidari R, Devonshire A L, Campbell B E, Dorrian S J, Oakeshott J G, Russell R J. Hydrolysis of pyrethroids by carboxylesterases from Lucilia cuprina and Drosophila melanogaster with active sites modified by in vitro mutagenesis. Insect Biochem Mol Biol. 2005 June; 35(6):597-609. Epub 2005 Mar. 31.
(64) Hesketh H, Lahive E, Horton A A, Robinson A G, Svendsen C, Rortais A, Dome J L, Baas J, Spurgeon D J, Heard M S. Extending standard testing period in honeybees to predict lifespan impacts of pesticides and heavy metals using dynamic energy budget modelling. Sci Rep. 2016 Dec. 20; 6:37655.
(65) Hladik, M. L., Smalling, K. L., and Kuivila, K. M., Methods of analysis-Determination of pyrethroid insecticides in water and sediment using gas chromatography/mass spectrometry. U. G. Survey, Editor 2009.
(66) Hroncova Z, Havlik J, Killer J, Doskocil I, Tyl J, Kamler M, Titera D, Hakl J, Mrazek J, Bunesova V, Rada V. Variation in honey bee gut microbial diversity affected by ontogenetic stage, age and geographic location. PLoS One. 2015 Mar. 13; 10(3):e0118707.
(67) Hurwitz, I., Fieck, A., Read, A., Hillesland, H., Klein, N., Kang, A., Durvasula, R. (2011) Paratransgenic control of vector borne diseases. Int J Biol Sci 7, 1334-44. Review. (68) Hussain S1,2, Hartley C J3, Shettigar M2, Pandey G2. Bacterial biodegradation of neonicotinoid pesticides in soil and water systems. FEMS Microbiol Lett. 2016 December; 363(23). pii: fnw252.
(69) Ihara M, Buckingham S D, Matsuda K, Sattelle D B. Modes of action, resistance and toxicity of insecticides targeting nicotinic acetylcholine receptors. Curr Med Chem. 2017 Feb. 6. doi:10.2174/0929867324666170206142019. [Epub ahead of print].
(70) Itoh, H., Aita, M., Nagayama, A., Meng, X. Y., Kamagata, Y., Navarro, R., Hori, T., Ohgiya, S., Kikuchi, Y. (2014) Evidence of Environmental and Vertical Transmission of Burkholderia Symbionts in the Oriental Chinch Bug, Cavelerius saccharivorus (Heteroptera: Blissidae) Journal Appl Environ. Microbiol 80, 5974-5983.
(71) Iyer R, Iken B, Damania A. A comparison of organophosphate degradation genes and bioremediation applications. Environ Microbiol Rep. 2013 December; 5(6):787-98.
(72) Jia, H., Dai, P., Geng, L., Jack, C. J., Li, Y., Wu, Y., Diao, Q., Ellis, J. D. 2017. No effect of Bt Crylle toxin on bacterial diversity in the midgut of the Chinese honey bee, Apis cerana cerana (Hymenoptera, Apidae). Scientific Reports 7: 41688.
(73) Johnson R M, Wen Z, Schuler M A, Berenbaum M R. Mediation of pyrethroid insecticide toxicity to honey bees (Hymenoptera: Apidae) by cytochrome P450 monooxygenases. J Econ Entomol. 2006 August; 99(4):1046-50.
(74) Johnson R M, Ellis M D, Mullin C A, Frazier M. Pesticides and honey bee toxicity in the United States. (2012) In: Honey Bee Colony Health: Challenges and Sustainable Solutions (Contemporary Topics in Entomology) Diana Sammataro (Editor), Jay A. Yoder (Editor).
(75) Kikuchi Y, Hayatsu M, Hosokawa T, Nagayama A, Tago K, Fukatsu T. Symbiont-mediated insecticide resistance. Proc Natl Acad Sci USA. 2012 May 29; 109(22):8618-22.
(76) Klein A M, Steffan-Dewenter I, Tscharntke T. Fruit set of highland coffee increases with the diversity of pollinating bees. Proc Biol Sci. 2003 May 7; 270(1518):955-61.
(77) Klein S, Cabirol A, Devaud J M, Barron A B, Lihoreau M. Why Bees Are So Vulnerable to Environmental Stressors. Trends Ecol Evol. 2017 April; 32(4):268-278.
(78) Kovach M E1, Elzer P H, Hill D S, Robertson G T, Farris M A, Roop R M 2nd, Peterson K M. Four new derivatives of the broad-host-range cloning vector pBBR1MCS, carrying different antibioticresistance cassettes. Gene. 1995 Dec. 1; 166(1):175-6.
(79) Kristich C J, Manias D A, Dunny G M. Development of a method for markerless genetic exchange in Enterococcus faecalis and its use in construction of a srtA mutant. Appl Environ Microbiol. 2005 October; 71(10):5837-49.
(80) Kwong W K, Moran N A. Cultivation and characterization of the gut symbionts of honey bees and bumble bees: description of Snodgrassella alvi gen. nov., sp. nov., a member of the family Neisseriaceae of the Betaproteobacteria, and Gilliamella apicola gen. nov., sp. nov., a member of Orbaceae fam. nov., Orbales ord. nov., a sister taxon to the order ‘Enterobacteriales’ of the Gammaproteobacteria. Int J Syst Evol Microbiol. 2013 June; 63(Pt 6):2008-18.
(81) Kwong W K, Engel P, Koch H, Moran N A. Genomics and host specialization of honey bee and bumble bee gut symbionts. Proc Natl Acad Sci USA. 2014 Aug. 5; 111(31):11509-14.
(82) Kwong W K, Moran N A. Gut microbial communities of social bees. Nat Rev Microbiol. 2016 June; 14(6):374-84.
(83) Kwong W K, Mancenido A L, Moran N A. Immune system stimulation by the native gut microbiota of honey bees. R Soc Open Sci. 2017 Feb. 8; 4(2):170003.
(84) Le Conte Y, Brunet J L, McDonnel C, Dussaubat C, Alaux C., Interactions between risk factors in honey bees. (2012) In: Honey Bee Colony Health: Challenges and Sustainable Solutions (Contemporary Topics in Entomology) Diana Sammataro (Editor), Jay A. Yoder (Editor).
(85) Lee, F. J., Rusch, D. B., Stewart, F. J., Mattila H R, Newton, I. L. (2015) Saccharide breakdown and fermentation by the honey bee gut microbiome. Environ Microbiol 17, 796-815.
(86) Len Foote H., (1957) Possible use of microorganisms in Synthetic Bee Bread Production. Amer. Bee J 97, 476-477.
(87) Li H, Cheng F, Wei Y, Lydy M J, You J. Global occurrence of pyrethroid insecticides in sediment and the associated toxicological effects on benthic invertebrates: An overview. J Hazard Mater. 2017 Feb. 15; 324(Pt B):258-271.
(88) Li Y, Liu J, Lu M, Ma Z, Cai C, Wang Y, Zhang X. Bacterial Expression and Kinetic Analysis of Carboxylesterase 001D from Helicoverpa armigera. Int J Mol Sci. 2016 Apr. 2; 17(4):493.
(89) Liu X, Liang M, Liu Y, Fan X. Directed evolution and secretory expression of a pyrethroidhydrolyzing esterase with enhanced catalytic activity and thermostability. Microb Cell Fact. 2017 May 11; 16(1):81.
(90) Ludvigsen J, Rangberg A, Avershina E, Sekelja M, Kreibich C, Amdam G, Rudi K. Shifts in the Midgut/Pyloric Microbiota Composition within a Honey Bee Apiary throughout a Season. Microbes Environ. 2015; 30(3):235-44.
(91) Maddaloni M., Forlani F., Balmas V., Donini G., Stasse L., Corazza L., Motto M., Increased tolerance to the fungal pathogen Rhizoctonia solani in transgenic tobacco expressing the maize ribosome inactivating protein b-32. 1997 Transgenic Research 6: 393-402.
(92) Maddaloni M, Staats H F, Mierzejewska D, Hoyt T, Robinson A, Callis G, Kozaki S, Kiyono H, McGhee J R, Fujihashi K, Pascual D W. Mucosal vaccine targeting improves onset of mucosal and systemic immunity to botulinum neurotoxin A. J Immunol. 2006 Oct. 15; 177(8): 5524-32.
(93) Maddaloni M, Hoffman C, Pascual D W. 2014 Paratransgenesis feasibility in the honeybee (Apis mellifera) using Fructobacillus fructosus commensal. J Appl Microbiol 117, 1572-84.
(94) Maddaloni M, Kochetkova I, Jun S, Callis G, Thornburg T, Pascual D W. (2015) Milk-based nutraceutical for treating autoimmune arthritis via the stimulation of IL-10- and TGF-β-producing CD39+ regulatory T cells. PLoS One 10:e0117825.
(95) Maddaloni M., D. W. Pascual. Isolation of oxalotrophic bacteria associated with varroa (Varroa destructor) mites. 2015 Letters in Applied Microbiology. Lett Appl Microbiol. 2015 61(5):411-417.
(96) Maddaloni, M., Nguyen, C. Q., Pascual, D. W. Expression of heterologous oxalate decarboxylase in honey bee (Apis mellifera) commensals Fructobacillus fructosus and Lactobacillus kunkeei. (submitted)
(97) Martinson V G, Danforth B N, Minckley R L, Rueppell O, Tingek S, Moran N A. A simple and distinctive microbiota associated with honey bees and bumble bees. Mol Ecol. 2011 February; 20(3):619-28
(98) Martinson V G, Moy J, Moran N A. Establishment of characteristic gut bacteria during development of the honeybee worker. Appl Environ Microbiol. 2012 April; 78(8):2830-40.
(99) Mattila H R, Rios D, Walker-Sperling V E, Roeselers G, Newton I L. Characterization of the active microbiotas associated with honey bees reveals healthier and broader communities when colonies are genetically diverse. PLoS One. 2012; 7(3):e32962.
(100) Meijer B J, Dieleman L A. Probiotics in the treatment of human inflammatory bowel diseases: update 2011. J Clin Gastroenterol. 2011 November; 45 Suppl:S139-44.
(101) Mohr K I, Tebbe C C. Diversity and phylotype consistency of bacteria in the guts of three bee species (Apoidea) at an oilseed rape field. Environ Microbiol. 2006 February; 8(2):258-72.
(102) Mullin, C. A., Frazier, M., Frazier, J. L., Ashcraft, S., Simonds, R., vanEngelsdorp, D., and Pettis, J. S., High Levels of Miticides and Agrochemicals in North American Apiaries: Implications for Honey Bee Health. Plos One, 5(3) (2010).
(103) Nauen R, Tietjen K, Wagner K and Elbert A, Efficacy of plant metabolites of imidacloprid against Myzus persicae and Aphisgossypii (Homoptera: Aphididae). Pestic Sci 52:53-57 (1998)
(104) Nauen R, Reckmann U, Armborst S, Stupp H P a nd Elbert A, Whitefly-active metabolites of imidacloprid: biological efficacy and translocation in cotton plants. Pestic Sci 55:265-271 (1999).
(105) Ng T K, Gahan L R, Schenk G, 011 is DL. Altering the substrate specificity of methyl parathion hydrolase with directed evolution. Arch Biochem Biophys. 2015 May 1; 573:59-68.
(106) Ngai M, McDowell M A. The search for novel insecticide targets in the post-genomics era, with a specific focus on G-protein coupled receptors. Mem Inst Oswaldo Cruz. 2017 Jan. 1; 112(1):1-7.
(107) Olofsson T C, Alsterfjord M, Nilson B, Butler E, Vásquez A. Lactobacillus apinorum sp. nov., Lactobacillus mellifer sp. nov., Lactobacillus mellis sp. nov., Lactobacillus melliventris sp. nov., Lactobacillus kimbladii sp. nov., Lactobacillus helsingborgensis sp. nov. and Lactobacillus kullabergensis sp. nov., isolated from the honey stomach of the honeybee Apis mellifera. Int J Syst Evol Microbiol. 2014 September; 64(Pt 9):3109-19.
(108) O'Flaherty S, Klaenhammer T R. Multivalent Chromosomal Expression of the Clostridium botulinum Serotype A Neurotoxin Heavy-Chain Antigen and the Bacillus anthracis Protective Antigen in Lactobacillus acidophilus. Appl Environ Microbiol. 2016 Sep. 30; 82(20):6091-6101.
(109) Ohbayashi T, Takeshita K, Kitagawa W, Nikoh N, Koga R, Meng X Y, Tago K, Hori T, Hayatsu M, Asano K, Kamagata Y, Lee B L, Fukatsu T, Kikuchi Y. Insect's intestinal organ for symbiont sorting. Proc Natl Acad Sci USA. 2015 Sep. 15; 112(37):E5179-88.
(110) Pain, J., Maugenet, J. (1966) Recherches biochimiques et physiologiques sur le pollen emmagasine' par les abeilles. Ann, Abeille 9, 209-236.
(111) Philippe N, Alcaraz J P, Coursange E, Geiselmann J, Schneider D. Improvement of pCVD442, a suicide plasmid for gene allele exchange in bacteria. Plasmid. 2004 May; 51(3):246-55.
(112) Phillips J P, Xin J H, Kirby K, Milne C P Jr, Krell P, Wild J R. Transfer and expression of an organophosphate insecticide-degrading gene from Pseudomonas in Drosophila melanogaster. Proc Natl Acad Sci USA. 1990 October; 87(20):8155-9.
(113) Potts S G, Imperatriz-Fonseca V, Ngo H T, Aizen M A, Biesmeijer J C, Breeze T D, Dicks L V, Garibaldi L A, Hill R, Settele J, Vanbergen A J. Safeguarding pollinators and their values to human well-being. Nature. 2016 Dec. 8; 540(7632):220-229
(114) Powell J E, Martinson V G, Urban-Mead K, Moran N A. Routes of Acquisition of the Gut Microbiota of the Honey Bee Apis mellifera. Appl Environ Microbiol. 2014 December; 80(23): 7378-87.
(115) Prakash, O., Shouche, Y., Jangid, K., Kostka, J. E. (2013) Microbial cultivation and the role of microbial resource centers in the omics era. Appl Microbiol Biotechnol. 97, 51-62.
(116) Pritchett M A, Zhang J K, Metcalf W W. Development of a markerless genetic exchange method for Methanosarcina acetivorans C2A and its use in construction of new genetic tools for methanogenic archaea. Appl Environ Microbiol. 2004 March; 70(3):1425-33.
(117) Purg M, Pabis A, Baier F, Tokuriki N, Jackson C, Kamerlin S C. Probing the mechanisms for the selectivity and promiscuity of methyl parathion hydrolase. Philos Trans A Math Phys Eng Sci. 2016 Nov. 13; 374 (2080). pii: 20160150.
(118) Rader R, Bartomeus I, Garibaldi L A, Garratt M P, Howlett B G, Winfree R, Cunningham S A, Mayfield M M, Arthur A D, Andersson G K, Bommarco R, Brittain C, Carvalheiro L G, Chacoff N P, Entling M H, Foully B, Freitas B M, Gemmill-Herren B, Ghazoul J, Griffin S R, Gross C L, Herbertsson L, Herzog F, Hipólito J, Jaggar S, Jauker F, Klein A M, Kleijn D, Krishnan S, Lemos C Q, Lindström S A, Mandelik Y, Monteiro V M, Nelson W, Nilsson L, Pattemore D E, Pereira Nde O, Pisanty G, Potts S G, Reemer M, Rundlöf M, Sheffield C S, Scheper J, Schüepp C, Smith H G, Stanley D A, Stout J C, Szentgyörgyi H, Taki H, Vergara C H, Viana B F, Woyciechowski M. Non-bee insects are important contributors to global crop pollination. Proc Natl Acad Sci USA. 2016 Jan. 5; 113(1):146-51.
(119) Rangberg A, Diep D B, Rudi K, Amdam G V. Paratransgenesis: an approach to improve colony health and molecular insight in honey bees (Apis mellifera)? Integr Comp Biol. 2012 July; 52(1):89-99.
(120) Rangberg A, Mathiesen G, Amdam G V, Diep D B. The paratransgenic potential of Lactobacillus kunkeei in the honey bee Apis mellifera. Benef Microbes. 2015; 6(4):513-23.
(121) Ranson H, Claudianos C, Ortelli F, Abgrall C, Hemingway J, Sharakhova M V, Unger M F, Collins F H, Feyereisen R. Evolution of supergene families associated with insecticide resistance. Science. 2002 Oct. 4; 298(5591):179-81.
(122) Retschnig G, Williams G R, Odemer R, Boltin J, Di Poto C, Mehmann M M, Retschnig P, Winiger P, Rosenkranz P, Neumann P Effects, but no interactions, of ubiquitous pesticide and parasite stressors on honey bee (Apis mellifera) lifespan and behaviour in a colony environment. Environ Microbiol. 2015 November; 17(11):4322-31.
(123) Ried J L, Collmer A. An nptI-sacB-sacR cartridge for constructing directed, unmarked mutations in gram-negative bacteria by marker exchange-eviction mutagenesis. Gene. 1987; 57(2-3):239-46.
(124) Ruiz-Argueso, T., Rodriguez-Navarro, A. (1975) Microbiology of ripening honey Appl Microbiol 30, 893-6.
(125) Russell R J, Scott C, Jackson C J, Pandey R, Pandey G, Taylor M C, Coppin C W, Liu J W, Oakeshott J G. The evolution of new enzyme function: lessons from xenobiotic metabolizing bacteria versus insecticide-resistant insects. Evol Appl. 2011 March; 4(2):225-48.
(126) Ryffel G U. Transgene flow: facts, speculations and possible countermeasures. GM Crops Food. 2014; 5(4):249-58.
(127) Sabree Z L, Hansen A K, Moran N A. Independent studies using deep sequencing resolve the same set of core bacterial species dominating gut communities of honey bees. PLoS One. 2012; 7(7):e41250.
(128) Saez-Lara M J, Gomez-Llorente C, Plaza-Diaz J, Gil A. The role of probiotic lactic acid bacteria and bifidobacteria in the prevention and treatment of inflammatory bowel disease and other related diseases: a systematic review of randomized human clinical trials. Biomed Res Int. 2015; 2015:505878.
(129) Sanchez-Bayo F, Goka K. Pesticide residues and bees—a risk assessment. PLoS One. 2014 Apr. 9; 9(4):e94482.
(130) Sánchez-Bayo F, Goulson D, Pennacchio F, Nazzi F, Goka K, Desneux N. Are bee diseases linked to pesticides? Environ Int. 2016 April-May; 89-90:7-11.
(131) Schmehl, D. R., Tome, H. V. V., Mortensen, A. N., Martins, G. F., Ellis, J. D. 2016. Improved protocol for the in vitro rearing of Apis mellifera workers. Journal of Apicultural Research 55(2): 113-129.
(132) Schmuck R1, Stadler T, Schmidt H W. Field relevance of a synergistic effect observed in the laboratory between an EBI fungicide and a chloronicotinyl insecticide in the honeybee (Apis mellifera L, Hymenoptera). Pest Manag Sci. 2003 March; 59(3):279-86.
(133) Scott J G. Evolution of resistance to pyrethroid insecticides in Musca domestica. Pest Manag Sci. 2017 April; 73(4):716-722.
(134) Sethi A, Delatte J, Foil L, Husseneder C. Protozoacidal Trojan-Horse: use of a ligand-lytic peptide for selective destruction of symbiotic protozoa within termite guts. PLoS One. 2014 Sep. 8; 9(9):e106199.
(135) Sih A, Bell A M, Kerby J L. Two stressors are far deadlier than one. Trends Ecol Evol. 2004 June; 19(6):274-6.
(136) Simon M C, Strassburger K, Nowotny B, Kolb H, Nowotny P, Burkart V, Zivehe F, Hwang J H, Stehle P, Pacini G, Hartmann B, Holst J J, MacKenzie C, Bindels L B, Martinez I, Walter J, Henrich B, Schloot N C, Roden M. Intake of Lactobacillus reuteri improves incretin and insulin secretion in glucose-tolerant humans: a proof of concept. Diabetes Care. 2015 October; 38(10): 1827-34.
(137) Simpson D J, Fry J C, Rogers H J, Day M J. Transformation of Acinetobacter baylyi in non-sterile soil using recombinant plant nuclear DNA. Environ Biosafety Res. 2007 January-June; 6(1-2):101-12.
(138) Singh B K. Organophosphorus-degrading bacteria: ecology and industrial applications. Nat Rev Microbiol. 2009 February; 7(2):156-64.
(139) Singh B, Kaur J, Singh K. Microbial degradation of an organophosphate pesticide, malathion. Crit Rev Microbiol. 2014 May; 40(2):146-54.
(140) Suchail S, De Sousa G, Rahmani R, Belzunces L P. In vivo distribution and metabolisation of 14Cimidacloprid in different compartments of Apis mellifera L. Pest Manag Sci. 2004 November; 60(11):1056-62.
(141) Tago, K., Okubo, T., Itoh, H., Kikuchi, Y., Hori, T., Sato, Y., Nagayama, A., Hayashi, K., Ikeda, S., Hayatsu, M. (2015) Insecticide-degrading burkholderia symbionts of the stinkbug naturally occupy various environments of sugarcane fields in a southeast island of Japan. Microbes Environ. 30, 29-36.
(142) Teese M G, Campbell P M, Scott C, Gordon K H, Southon A, Hovan D, Robin C, Russell R J, Oakeshott J G Gene identification and proteomic analysis of the esterases of the cotton bollworm, Helicoverpa armigera. Insect Biochem Mol Biol. 2010 January; 40(1):1-16.
(143) Toiviainen A, Jalasvuori H, Lahti E, Gursoy U, Salminen S, Fontana M, Flannagan S, Eckert G, Kokaras A, Paster B, Söderling E. Impact of orally administered lozenges with Lactobacillus rhamnosus GG and Bifidobacterium animalis subsp. lactis BB-12 on the number of salivary mutans streptococci, amount of plaque, gingival inflammation and the oral microbiome in healthy adults. Clin Oral Investig. 2015 January; 19(1):77-83.
(144) Turrini A, Sbrana C, Giovannetti M. Belowground environmental effects of transgenic crops: a soil microbial perspective. Res Microbiol. 2015 April; 166(3):121-31.
(145) Ueki T, Inouye S, Inouye M. Positive-negative KG cassettes for construction of multi-gene deletions using a single drug marker. Gene. 1996 Dec. 12; 183(1-2):153-7.
(146) van Engelsdorp, D., Hayes, J. Jr, Underwood, R. M., Pettis, J. (2008) A survey of honey bee colony losses in the U.S., fall 2007 to spring. PLoS One 3:e4071.
(147) van Engelsdorp, D. and Meixner, M. D. (2010). A historical review of managed honey bee populations in Europe and the United States and the factors that may affect them. J lnvertebr Pathol 103, S80-95.
(148) vanEngelsdorp, D., Tarpy, D. R., Lengerich, E. J., Pettis, J. S. (2013). Idiopathic brood disease syndrome and queen events as precursors of colony mortality in migratory beekeeping operations in the eastern United States. Prev Vet Med. 108, 225-33.
(149) Vásquez A, Olofsson T C, Sammataro D. A scientific note on the lactic acid bacterial flora in honeybees in the USA—A comparison with bees from Sweden Apidologie 40 (2009) 26-28.
(150) Vásquez, A., Forsgren, E, Frie, s I., Paxton, R. J., Flaberg, E., Szekely, L., Olofsson, T. C. (2012) Symbionts as major modulators of insect health: lactic acid bacteria and honeybees PLoS One 7:e33188.
(151) Wang B Z, Guo P, Hang B J, Li L, He J, Li S P. Cloning of a novel pyrethroid-hydrolyzing carboxylesterase gene from Sphingobium sp. strain JZ-1 and characterization of the gene product. Appl Environ Microbiol. 2009 September; 75(17):5496-500.
(152) Wang L L, Huang Y, Lu X P, Jiang X Z, Smagghe G, Feng Z J, Yuan G R, Wei D, Wang J J. Overexpression of two α-esterase genes mediates metabolic resistance to malathion in the oriental fruit fly, Bactrocera dorsalis (Hendel). Insect Mol Biol. 2015 August; 24(4):467-79.
(153) Werren J H. Symbionts provide pesticide detoxification. Proc Natl Acad Sci USA. 2012 May 29; 109(22):8364-5.
(154) Weston D P, Jackson C J. Use of engineered enzymes to identify organophosphate and pyrethroidrelated toxicity in toxicity identification evaluations. Environ Sci Technol. 2009 Jul. 15; 43(14):5514-20.
(155) Wiest, L., Bulete, A., Giroud, B., Fratta, C., Amic, S., Lambert, O., Pouliquen, H., and Arnaudguilhem, C., Multi-residue analysis of 80 environmental contaminants in honeys, honeybees and pollens by one extraction procedure followed by liquid and gas chromatography coupled with mass spectrometric detection. J Chromatogr A, 1218(34): 5743-56 (2011).
(156) Wollman E. On raising sterile flies. Contribution to the knowledge of the role of microbes in the digestive tract. Ann Inst Pasteur (Paris) 1911; 25:79-88.
(157) Wu K, Hoy M A. The Glutathione-S-Transferase, Cytochrome P450 and Carboxyl/Cholinesterase Gene Superfamilies in Predatory Mite Metaseiulus occidentalis. PLoS One. 2016 Jul. 28; 11(7):e0160009.
(158) Wu P C, Liu Y H, Wang Z Y, Zhang X Y, Li H, Liang W Q, Luo N, Hu J M, Lu J Q, Luan T G, Cao L X. Molecular cloning, purification, and biochemical characterization of a novel pyrethroid-hydrolyzing esterase from Klebsiella sp. strain ZD112. J Agric Food Chem. 2006 Feb. 8; 54(3):836-42.
(159) Wu S, Yang Y, Yuan G, Campbell P M, Teese M G, Russell R J, Oakeshott J G, Wu Y. Overexpressed esterases in a fenvalerate resistant strain of the cotton bollworm, Helicoverpa armigera. Insect Biochem Mol Biol. 2011 January; 41(1):14-21.
(160) Wu Y J, Wu W F, Hung C W, Ku M S, Liao P F, Sun H L, Lu K H, Sheu J N, Lue K H. Evaluation of efficacy and safety of Lactobacillus rhamnosus in children aged 4-48 months with atopic dermatitis: An 8-week, double-blind, randomized, placebo-controlled study. J Microbiol Immunol Infect. 2015 Nov. 27. pii: S1684-1182(15)00898-1.
(161) Xi J, Pan Y, Bi R, Gao X, Chen X, Peng T, Zhang M, Zhang H, Hu X, Shang Q. Elevated expression of esterase and cytochrome P450 are related with lambda-cyhalothrin resistance and lead to cross resistance in Aphis glycines Matsumura. Pestic Biochem Physiol. 2015 February; 118:77-81.
(162) Xie J, Zhao Y, Zhang H, Liu Z, Lu Z. Improving methyl parathion hydrolase to enhance its chlorpyrifos-hydrolysing efficiency. Lett Appl Microbiol. 2014 January; 58(1):53-9.
(163) Yang X, Becker T, Walters N, Pascual D W. Deletion of znuA virulence factor attenuates Brucella abortus and confers protection against wild-type challenge. Infect Immun. 2006 July; 74(7):3874-9.
(164) Zhang R, Cui Z, Zhang X, Jiang J, Gu J D, Li S. Cloning of the organophosphorus pesticide hydrolase gene clusters of seven degradative bacteria isolated from a methyl parathion contaminated site and evidence of their horizontal gene transfer. Biodegradation. 2006 October; 17(5):465-72.
(165) Zhao R, Han R, Qiu X, Yan X, Cao L, Liu X. Cloning and heterologous expression of insecticidal protein-encoding genes from Photorhabdus luminescens TT01 in Enterobacter cloacae for termite control. Appl Environ Microbiol. 2008 December; 74(23):7219-26.
(166) Zhu Y. C. A. K. Dowdy, J. E. Baker Detection of single-base substitution in an esterase gene and its linkage to malathion resistance in the parasitoid Anisopteromalus calandrae (Hymenoptera: Pteromalidae) Pestic. Sci., 55 (1999), pp. 398-404.
(167) Zhu Y C, Yao J, Adamczyk J, Luttrell R. Synergistic toxicity and physiological impact of imidacloprid alone and binary mixtures with seven representative pesticides on honey bee (Apis mellifera). PLoS One. 2017 May 3; 12(5):e0176837.
(168) Zuo Z, Gong T, Che Y, Liu R, Xu P, Jiang H, Qiao C, Song C, Yang C. Engineering Pseudomonas putida KT2440 for simultaneous degradation of organophosphates and pyrethroids and its application in bioremediation of soil. Biodegradation. 2015 June; 26(3):223-33.
(169) Cox-Foster D L, Conlan S, Holmes E C, Palacios G, Evans J D, Moran N A, Quan P L, Briese T, Hornig M, Geiser D M, Martinson V, vanEngelsdorp D, Kalkstein A L, Drysdale A, Hui J, Zhai J, Cui L, Hutchison S K, Simons J F, Egholm M, Pettis J S, Lipkin W I. A metagenomic survey of microbes in honey bee colony collapse disorder. Science. 2007 Oct. 12; 318(5848):283-7.
(170) Yang X, Becker T, Walters N, Pascual D W. Deletion of znuA virulence factor attenuates Brucella abortus and confers protection against wild-type challenge. Infect Immun. 2006 July; 74(7):3874-9.
(171) Al-Ghamdi A, Ali Khan K, Javed Ansari M, Almasaudi S B, Al-Kahtani S. Effect of gut bacterial isolates from Apis mellifera jemenitica on Paenibacillus larvae infected bee larvae. Saudi J Biol Sci. 2018 February; 25(2):383-387.
(172) Arredondo D, Castelli L, Porrini M P, Garrido P M, Eguaras M J, Zunino P, Antünez K. Lactobacillus kunkeei strains decreased the infection by honey bee pathogens Paenibacillus larvae and Nosema ceranae. Benef Microbes. 2018 Feb. 27; 9(2):279-290.
(173) Audisio M C, Benitez-Ahrendts M R. Lactobacillus johnsonii CRL1647, isolated from Apis mellifera L. bee-gut, exhibited a beneficial effect on honeybee colonies. Benef Microbes. 2011 March; 2(1):29-34.
(174) Eibes G, Arca-Ramos A, Feijoo G, Lema J M, Moreira M T. Enzymatic technologies for remediation of hydrophobic organic pollutants in soil. Appl Microbiol Biotechnol. 2015 November; 99(21):8815-29.
(175) Evans J D, Cook S C. Genetics and physiology of Varroa mites. Curr Opin Insect Sci. 2018 April; 26:130-135.
(176) Kadisch M, Willrodt C, Hillen M, Bühler B, Schmid A. Maximizing the stability of metabolic engineering-derived whole-cell biocatalysts. Biotechnol J. 2017 August; 12(8).
(177) Leonard S P, Perutka J, Powell J E, Geng P, Richhart D D, Byrom M, Kar S, Davies B W, Ellington A D, Moran N A, Barrick J E. Genetic Engineering of Bee Gut Microbiome Bacteria with a Toolkit for Modular Assembly of Broad-Host-Range Plasmids. ACS Synth Biol. 2018 May 18; 7(5):1279-1290.
(178) Martin S J. Acaricide (prethroid) resistance in Varroa destructor. Bee world 2004 85(4): 67-69.
(179) Polakovič M, Švitel J, Bučko M, Filip J, Neděla V, Ansorge-Schumacher M B, Gemeiner P. Progress in biocatalysis with immobilized viable whole cells: systems development, reaction engineering and applications. Biotechnol Lett. 2017 May; 39(5):667-683.
(180) Rayu S, Karpouzas D G, Singh B K. Emerging technologies in bioremediation: constraints and opportunities. Biodegradation. 2012 November; 23(6):917-26.
(181) Sabaté D C, Cruz M S, Benitez-Ahrendts M R, Audisio M C. Beneficial Effects of Bacillus subtilis subsp. subtilis Mori2, a Honey-Associated Strain, on Honeybee Colony Performance. Probiotics Antimicrob Proteins. 2012 March; 4(1):39-46.
(182) Sanchez-Bayo F, Goka K. Pesticide residues and bees—a risk assessment. PLoS One. 2014 Apr. 9; 9(4):e94482.
(183) Soderlund D M, Clark J M, Sheets L P, Mullin L S, Piccirillo V J, Sargent D, Stevens J T, Weiner M L. Mechanisms of pyrethroid neurotoxicity: implications for cumulative risk assessment. Toxicology. 2002 Feb. 1; 171(1):3-59.
(184) Spök A, Arvanitakis G, McClung G. Status of microbial based cleaning products in statutory regulations and ecolabelling in Europe, the USA, and Canada. Food Chem Toxicol. 2018 June; 116(Pt A):10-19.
(185) Stadlmair L F, Letzel T, Drewes J E, Grassmann J. Enzymes in removal of pharmaceuticals from wastewater: A critical review of challenges, applications and screening methods for their selection. Chemosphere. 2018 August; 205:649-661.
(186) Tang W, Wang D, Wang J, Wu Z, Li L, Huang M, Xu S, Yan D. Pyrethroid pesticide residues in the global environment: An overview. Chemosphere. 2018 January; 191:990-1007.
(187) Tomlin C D S (2009) The e-Pesticide Manual. In: Tomlin C D S, editor. 12 ed. Surrey, U.K.: British Crop Protection Council.
(188) Microbiome Therapeutics and Diagnostics Market, 2017-2030. https://www.businesswire.com/news/home/20170829005566/en/Microbiome-Therapeutics-Diagnostics-Market-2017-2030—Research.

### Example 3

Embodiments of coding sequences of recombinant carboxylesterases as described herein are listed below.

DQ677027.1 Stenotrophomonas sp. YC-1 methyl
parathion hydrolase (mpd) gene
SEQ ID NO: 1
CTCTAGAGATTGAATTCATATGCCCCTGAAGAACCGCTTGCTGGCCCGCC
TGTCCTGTGTTGCGGCCGTGGTGGCCGCCACGGCCGCCGTTGCACCGTTG
ACGCTGGTGTCCACCGCCCACGCCGCCGCACCGCAGGTGCGCACCTCGGC
CCCCGGCTACTACCGGATGCTGCTGGGCGACTTCGAAATCACCGCGCTGT
CGGACGGCACGGTGGCGCTGCCGGTCGACAAGCGGCTGAACCAGCCGGCC
CCGAAGACGCAGAGCGCGCTGGCCAAGTCCTTCCAGAAAGCGCCGCTCGA
AACCTCGGTCACCGGTTACCTCGTCAACACCGGCTCCAAGCTGGTGCTGG
TGGACACCGGCGCGGCCGGCCTGTTCGGCCCCACCCTGGGCCGGCTGGCG
GCCAACCTCAAGGCCGCAGGCTATCAGCCCGAGCAGGTCGACGAGATCTA
CATCACCCACATGCACCCCGACCACGTGGGCGGCTTGATGGTGGGTGAGC
AACTGGCGTTCCCGAACGCGGTGGTGCGTGCGGACCAGAAAGAAGCCGAT
TTCTGGCTCAGCCAGACCAACCTCGACAAGGCCCCGGACGACGAGAGCAA
AGGCTTCTTCAAAGGCGCCATGGCCTCGCTGAACCCCTATGTGAAGGCCG
GCAAGTTCAAGCCTTTCTCGGGGAACACCGACCTGGTGCCCGGCATCAAA
GCGCTGGCCAGCCACGGCCACACCCCGGGCCACACCACCTACGTGGTCGA
AAGCCAGGGGCAAAAGCTCGCCCTGCTCGGCGACCTGATACTCGTCGCCG
CGGTGCAGTTCGACGACCCCAGCGTCACGACCCAGCTCGACAGCGACAGC
AAGTCCGTCGCGGTGGAGCGCAAGAAGGCCTTCGCGGATGCCGCCAAGGG
CGGCTACCTGATCGCGGCGTCCCACCTGTCGTTCCCCGGCATCGGCCACA
TCCGCGCCGAAGGCAAGGGCTACCGTTTCGTGCCGGTGAACTACTCGGTC
GTCAACCCCAAGTGACTCGAGAATCAATCGTCGAC
FJ688006.1 Sphindobium wenxiniae strain JZ-1
pyrethoid hydrolase (pytH) gene
SEQ ID NO: 2
TGCGGCCTTATATCGGCGGCGGCCTGAACTGGACGATCATCTTCAAGACC
GAAGACGGCGTGCTGAAGGATTTCCGCACCAGCAATACCGTCGGTCCTGC
GATCACGGGCGGCATCGATGTGCCGCTCAACGAGCGGATCGGTGTGTTCG
GCGCGGCCAGCAAGGTCTGGACCAGCACCAACGCCCGCTTCCGGCTCGGC
ACGCCGAACGGCTTCGTGCCGGGTACGGCGCGCGTCCAGCTCAATCCCCT
CATCGCGCAGGTCGGCCTGCAATATCGCTTCTGATCCCCCCGGCTCGAAC
AGGAAGACATGTCATGACCGTCACCGATATCATCCTGATCCACGGCGCCT
TGAACCGCGGCGCCTGCTATGACGCGGTCGTCCCGCTTCTCGAAGCGCGC
GGCTACCGCGTCCATGCGCCCGACCTGACCGGCCATACGCCCGGCGATGG
CGGCCATTTGTCGGTCGTCGACATGGAGCATTATACCCGCCCAGTCGCTG
ACATCCTGGCACGGGCCGAGGGGCAGTCGATCCTTCTGGGGCACAGCTTG
GGCGGTGCATCCATCTCGTGGCTGGCGCAGCACCATCCCGACAAGGTGGC
CGGGCTGATCTACCTGACCGCGGTCCTCACCGCGCCCGGTATAACGCCGG
AAACCTTCGTCCTGCCCGGCGAGCCCAACCGGGGCACGCCGCACGCGCTG
GACCTGATCCAGCCGGTCGACGAGGGACGTGGGCTACAGGCGGATTTCTC
GCGACTGGAACGGCTCCGCGAAGTTTTCATGGGCGATTATCCCGGCGAGG
GAATGCCGCCTGCCGAACAGTTCATCCAGACCCAGTCGACCGTGCCCTTT
GGCACGCCCAATCCGATGGAGGGGCGCGCGCTGGAAATCCCACGCCTCTA
TATCGAGGCGCTGGACGATGTCGTGATTCCGATCGCCGTGCAGCGTCAGA
TGCAGAAGGAGTTCCCCGGTCCGGTCGCGGTCGTGTCGCTGCCGGCCAGC
CATGCGCCCTATTACTCGATGCCCGAACGGCTGGCCGAGGCGATCGCCGA
TTTCGCCGATGCCCCGGCCGAGTATCGCCAGACGGCGACGAAGGCTGGGC
CTGATCGACCAGCTGGAGCGGACGGGGGTCGAGCCGACCGAGCTGATCTA
CCGTGACGGGCTGTCCGGTGATCGCCTGGCGGCCCATGCGGTGCGCAAGG
GCGGTGCCTATCGCGATCGGTTCGGCGCGCCCTATCTCGGCATCCACCGT
GCCGATCTGCAGACGACGCTGGGCCGCGCGGTCGGGGCGGAGCATATTCA
TCTGGCGCACCGGCTGGAGCGGATCGAGACGGTGGGCGAACGGGTGGCGC
TCCACTTCGTGGGACGCGATCCCGTCGAGGCCGATATCGTCGTGGGTGCC
GACGGTGTTCGCTCCATCACCCGGCAATGGGTGGGCGGGCAGCCGCTGCG
TTATTCCGGGACGAGCGCGTTTCGCGGCATCGTGCCGGTCGAGCGGCTGG
ACGCTTTGCCCGATCCCCAGGCGATTCAGTTCTGGATCGGCCAGGACGCC
CATCTGCTGC
AAB67728.1 E3 [Lucilia cuprina]
SEQ ID NO: 3
MNFNVSLMEKLKWKIKCIENKFLNYRLTTNETVVAETEYGKVKGVKRLTV
YDDSYYSFEGIPYAQPPVGELRFKAPQRPTPWDGVRDCCNHKDKSVQVDF
ITGKVCGSEDCLYLSVYTNNLNPETKRPVLVYIHGGGFIIGENHRDMYGP
DYFIKKDVVLINIQYRLGALGFLSLNSEDLNVPGNAGLKDQVMALRWIKN
NCANFGGNPDNITVFGESAGAASTHYMMLTEQTRGLFHRGILMSGNAICP
WANTQCQHRAFTLAKLAGYKGEDNDKDVLEFLMKAKPQDLIKLEEKVLTL
EERTNKVMFPFGPTVEPYQTADCVLPKHPREMVKTAWGNSIPTMMGNTSY
EGLFFTSILKQMPMLVKELETCVNFVPSELADAERTAPETLEMGAKIKKA
HVTGETPTADNFMDLCSHIYFWFPMHRLLQLRFNHTSGTPVYLYRFDFDS
EDLINPYRIMRSGRGVKGVSHADELTYFFWNQLAKRMPKESREYKTIERM
TGIWIQFATTGNPYSNEIEGMENVSWDPIKKSDEVYKCLNISDELKMIDV
PEMDKIKQWESMFEKHRDLF

### Example 4

Described in the present example is the decontamination of the wax for recycling in the production of foundation (the sheets of wax which are inserted into the frames to invite honeybees to draw new honeycomb). Pesticides accumulate in beeswax of the honeycombs, which have to be discarded after, usually, three years. While most impurities such as propolis, feces and exuviae form larvae, can be removed (by melting the spent honeycombs in presence of water), pesticides contamination cannot. Accordingly, the honeycomb wax has to be discarded and it cannot be recycled to produce new. It is not desirable for beekeepers to trash old combs not only because the new foundation has a substantial costs, but also because it can contain pathogens (some of which are sporigenic Paenibacilli that cannot be eliminated unless the wax is autoclaved). Moreover commercial foundation, unless the fabled very, very expensive organic batches, is known to carry pesticides of its own. Beeswax melts between 62-64° C., therefore “regular” carboxylesterases (such as those described above) may not be suitable. On the contrary, carboxylesterases from thermophilic bacteria (for example SEQ. ID NOs. 4 and 5 below) can withstand and have optimum catalytic activities at higher temperatures (around 65-75° C. and remain active up to 90° C.) than carboxylesterases derived from other sources, which would make them ideal for this purposes. In embodiments, these enzymes can be immobilized on nonwoven textile-like matrices which can act as sieves to retain impurities (as mentioned above) and decontaminate the beeswax at the same time. Organophosphate carboxylesterases (phospotriesterases) received great attention because organophoshates, besides being insecticides, are warfare nerve agents.

### REFERENCES

1) Hawwa R, Aikens J, Turner R J, Santarsiero B D, Mesecar A D. Structural basis for thermostability revealed through the identification and characterization of a highly thermostable phosphotriesterase-like lactonase from Geobacillus stearothermophilus. Arch Biochem Biophys. 2009 Aug. 15; 488(2):109-20. doi: 10.1016/j.abb.2009.06.005. Epub 2009 Jul. 16.
2) Hawwa R, Larsen S D, Ratia K, Mesecar A D. Structure-based and random mutagenesis approaches increase the organophosphate-degrading activity of a phosphotriesterase homologue from Deinococcus radiodurans. J Mol Biol. 2009 Oct. 16; 393(1):36-57
3) Kallnik V, Bunescu A, Sayer C, Bräsen C, Wohlgemuth R, Littlechild J, Siebers B. Characterization of a phosphotriesterase-like lactonase from the hyperthermoacidophilic crenarchaeon Vulcanisaeta moutnovskia. J Biotechnol. 2014 Nov. 20; 190:11-7.
4) Porzio E, Merone L, Mandrich L, Rossi M, Manco G. A new phosphotriesterase from Sulfolobus acidocaldarius and its comparison with the homologue from Sulfolobus solfataricus. Biochimie. 2007 May; 89(5):625-36.
5) Merone L, Mandrich L, Porzio E, Rossi M, Müller S, Reiter G, Worek F, Manco G. Improving the promiscuous nerve agent hydrolase activity of a thermostable archaeal lactonase. Bioresour Technol. 2010 December; 101(23):9204-12.
6) Jacquet P, Hiblot J, Daudé D, Bergonzi C, Gotthard G, Armstrong N, Chabrière E, Elias M. Rational engineering of a native hyperthermostable lactonase into a broad spectrum phosphotriesterase. Sci Rep. 2017 Dec. 1; 7(1):16745.
7) Blatchford P A1, Scott C, French N, Rehm B H. Immobilization of organophosphohydrolase OpdA from Agrobacterium radiobacter by overproduction at the surface of polyester inclusions inside engineered Escherichia coli. Biotechnol Bioeng. 2012 May; 109(5):1101-8.
8) Gao Y1, Truong Y B, Cacioli P, Butler P, Kyratzis I L. Bioremediation of pesticide contaminated water using an organophosphate degrading enzyme immobilized on nonwoven polyester textiles. Enzyme Microb Technol. 2014 Jan. 10; 54:38-44.

SEQ ID NO 4: 3GTF_A
Deinococcus radiodurans phosphotriesterase,
Genbank accession# 3GTF_A. The amino acid
sequence “hhhhhhgdap ggah” of the sequence
listing below is derived from the expression
vector, and does not belong to the protein itself)
hhhhhhgdapggahmtaqtvtgavaaaqlgatlphehvifgypgyagdvt
lgpfdhaaalasctetarallargiqtvvdatpngcgrnpaflrevseat
glqilcatgfyyegggattyfkfrasIgdaeseiyemmrtevtegiagtg
iragvixlassrdaitpyeqlffraaarvqretgvpiithtqegqqgpqq
aelltslgadparimighmdgntdpayhretlrhgvsiafdriglqgmlg
tptdaerlsvlttllgegyadrlllshdsiwhwlgrppaipeaalpavkd
whplhisddilpdlrrrgiteeqvgqmtvgnparlfg
SEQ ID NO 5: WP_011278935
Sulpholobus acidocaldarius phosphotriesterase,
Genbank accession# WP_011278935.
mtkiplvgkgeispgemgftlihehlrvfsepvryqwphlynedeelkna
vnevktimsygvktivdptvmglgrdirfsekvvketginviaatglyty
tdlpfffngrsleeiaellihdikkgiqgtnnragfikvaadepgitrdv
erairaaaiaqketnvpiithsnahngtgleqqrilmeegvdpgrvligh
lgdtdnvdyikkiadkgsfvgldrygldlflpidkrnevllklikdgyld
rimvsqdycctidwgiakpeykpklapkwsmsliftdvipsikragvtde
qlhvifvknparlfs

### Example 5

Neonicotinoids are recalcitrant to biodegradation and, among them, imidacloprid and clothianidin are particularly persistent. The degradation pathways of neonicotinoids are not completely understood. At least some of them eventually are transformed into 6-chloronicotinic acid (6-CNA) which can be transformed into 6-hydroxinicotinic acid (6-HNA) by the cch2 gene product from Bradyrhizobiaceae 6G-6C strain (Ref. Shettigar 2012). In turn, 6-HNA can be funneled into the Kreb's cycle through different routes (Jimenez 2008, Shettigar 2012). In Pseudomonas putida KT2440 the pathway of 6-HNA degradation has been thoroughly dissected (Jimenez 2008).

Described herein is a neonicotinoid detoxifying strategy of assembling several parts into devices and combining them into a cassette-in, cassette-out flexible genetic system where the catabolic genes converting 6-CNA into 6-HNA and then into fumaric acid, which is an intermediate of the Kreb's cycle, can be paired into the same plasmid with appropriate transporters (FIG. 14). Genetic system(s) as described herein can confer to engineered bacteria the capability to metabolize at least some neonicotinoids.

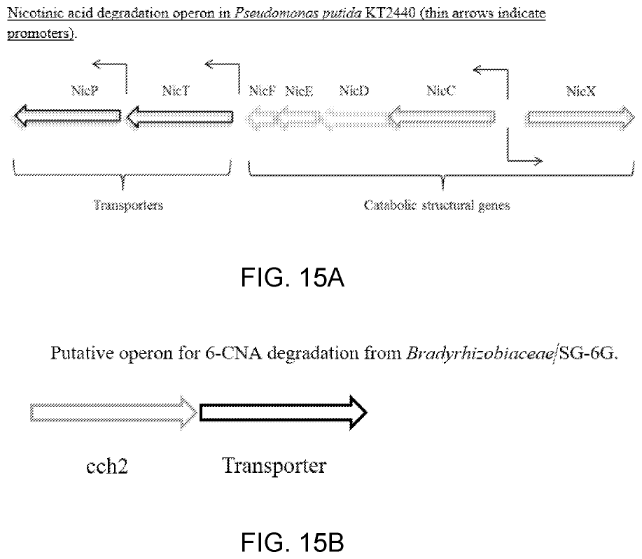

Described herein are genetic systems which, when introduced into a novel host, have the potential to confer the capability of degrading 6-CNA, thus creating a metabolically advantageous outlet for the mineralization of some intermediates of neonicotinoids catabolism. The P. putida 6-HNA operon was cloned by PCR using the genomic DNA as a template. The cch2 gene and its associated transporter were derived from a synthetic gene (FIGS. 15A and 15B).

Several constructs were made bearing different configurations of the above described genetic parts and transformed into E. coli BL-21 testing for the ability to grow on 6-HNA as a sole carbon source. Despite the extremely slow growth which cannot be measured by the increase in the OD600, the E. coli BL-21 cells, transformed with system were still alive, and capable of generating CFUs after 6 weeks incubation, whereas the control cells were not. This observation suggests that these constructs provide E. coli BL-21 limited, but sizeable, capability to use 6-HN to support metabolism.

The constructs and aspects thereof are shown in (FIG. 16 and FIG. 17). pGnMM89, 90, 91, 92, 93 express different combinations of the Pseudomonas putida KT2440 6-HNA operon under the control of the original promoters. pGnMM94 is identical to pGnMM93 except that the backbone carries a broad-range replicon for expression in a vast number of bacteria, including Pseudomonas otitidis (see population selected on cypermethrin). pGnMM95 expresses the Pseudomonas putida KT2440 catabolic genes under the inducible promoter. It has been designed to accept a BamHI-HindIII transporter(s) cassette including the Pseudomonas putida KT2440 transporters or the Bradyrhizobiaceae SG-6G cch2-transporter bi-cistronic unit.

### Example 6

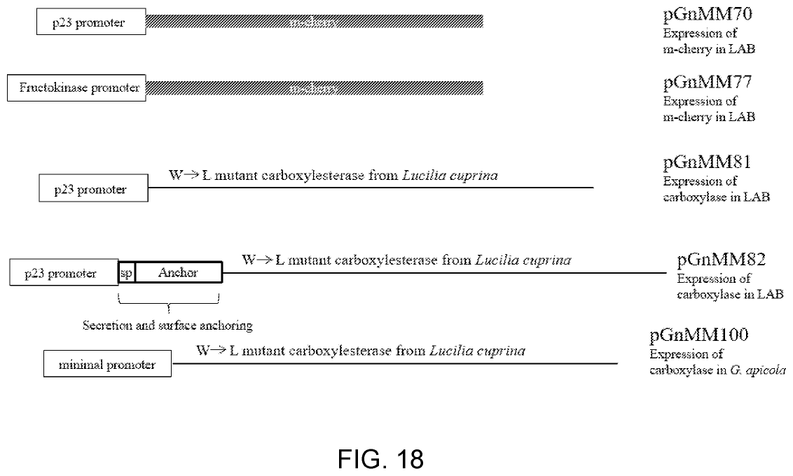

FIG. 18 illustrates embodiments of carboxylesterase expression vectors according to the present disclosure.

### Example 7

### Abstract

Oxalic acid, legally or brevi manu, is widely used to control phoretic Varroa destructor mites, a major drive of current honey bees' colony losses. Previous data suggests that varroa may become at least partially resistant to oxalic acid by associating with oxalotrophic bacteria, which use oxalates as a sole carbon source. Despite the efficacy against the varroa mites, oxalic acid is somewhat detrimental also to honey bees. In this composite scenario it is possible that honey bee commensals could be engineered to degrade oxalic acid. From a human probiotic strain of Bacillus subtilis, a gene encoding for oxalate decarboxylase was cloned, an enzyme that degrades both oxalates and oxalic acid to formate and CO₂, and was expressed in Lactobacillus kunkeei and Fructobacillus fructosus. Data show that these recombinant strains robustly degrade calcium oxalate. These strains will assist for a better understanding of the complex dynamics of oxalic acid inside the hive, and they may allow for precision interventions to shelter honey bees from the side-effects of miticides treatments. More importantly, this investigation provides a proof-of concept for the bioremediation of more toxic organic compounds inside the hive.

### Introduction

The current economic added-value of honey bees as crops pollinators, it is estimated in the tens of billions of dollars worldwide, thus exceeding by two logs (Gallai et al. 2009; Calderone 2012) the value of honey, wax, propolis, and bee-venom combined (van Engelsdorp and Meixner 2010). In the past 20 years a steady, dramatic decline of honey bees in has occurred. These losses depend on the type of operation, location, and year, and average 20-30% annually (van Engelsdorp D, 2008). According to (The Bee Informed Partnership), a non-profit organization connected to academic institutions, winter and summer combined losses currently range between 35-45%. The total cost of colony losses, the cost for their replacement and the loss of income combined, are likely to exceed hundreds of million annually. Scientific and regulatory institutions agree on these estimates (USDA, European Parliament).

Losses are in part attributed to the so-called colony collapse disorder (CCD), an umbrella name which defines a syndrome more than a specific disease, as well as to other ‘disappearing diseases’ (Cornman et al. 2012; van Engelsdorp et al. 2013). The causes of CCD and related diseases are poorly understood. Three stressors play major role in colony losses. 1) lack of a steady food supply throughout the entire year, 2) transfer of pathogens and parasites across the globe, 3) widespread use of pesticides. (Genersh 2010; Genersh and Aubert 2010; Evans and Schwartz 2011; Cornman et al. 2012; Becher 2013; Derecka et al. 2013). (Goulson et al. 2015 and references therein).

There is also a substantial agreement that the hemophagous honey bee mite Varroa destructor is a major drive of colony losses (Rosenkranz et al. 2009) and, while breeding aimed at improving honey bee resistance to varroasis is showing good potential and promising result (Moritz 1994; DeGuzman et al. 2005; Rinderer et al. 2014; Lattorf et al. 2015, Sabahi et al. 2017), mite control by beekeepers mostly relies on the use of chemicals, whose regulation is subjected to substantial differences worldwide and over the course of time. Over the last decades, the most noted synthetic acaricides against V. destructor are the organophosphate coumaphos, the pyrethroids tau-fluvalinate, as well as the formamidine amitraz against which V. destructor eventually developed resistance (Rosenkranz et al. 2009; Kanga et al. 2010). Some essential oils are reported to be effective against V. destructor (DeGrandi-Hoffman et al. 2012; Umpierrez et al. 2013; Damiani et al. 2014). A third category of miticides is represented by organic acids, lactic, formic and oxalic, which are empirically believed to carry minimal danger of resistance (Rosenkranz et al. 2009. and references therein). The use of organic acids against varroa dawned in the mid-80s (Klepsch et al. 1984,) and their use preceded, and in some cases subsided and/or disregarded regulations by government agencies. In certain regions, including Europe, when the honey supers, the combs where the honey is stored, separated from the nest, are not deployed, oxalic acid is legal to use and it is remarkably effective against varroa. Despite the efficacy against the varroa mites, oxalic acid is also detrimental to honey bees (Aliano et al. 2006; Aliano and Ellis 2009) possibly by interfering with the microbiome composition (Diaz et al. 2019). Accordingly, it is recommended that no more than one treatment is applied per generation, which in the active season averages about 45 days. It has been previously shown that varroa associates with oxalotrophic bacteria (Maddaloni and Pascual 2015), an observation that may explain why beekeepers lament that in certain instances they need to increase the frequency of oxalic acid treatments. In this complex scenario the ability to modulate the fate of oxalic acid in the hive through the microbiota offers novel approaches to refine its use for the control of phoretic varroa. Oxalic acid is a highly oxidized molecule that combines with various cations to form an array of salts. Among them, calcium oxalate has received much attention as 75% of all kidney stones are composed primarily of calcium oxalate (Robijn et al. 2011). A probiotic Lactobacillus plantarum has been engineered to degrade oxalate (Anbazhagan et al 2013,) and successfully used in a rat model of hyperoxaluria (Sasikumar et al 2014). In the present example the degradation of oxalate can be mediated by oxalate decarboxylase (OXDC, EC 4.1.1.2), an enzyme that catalyzes the oxidization of both oxalate and oxalic acid to formate and CO₂, both being volatile compounds (Tanner et al. 2001, Makela et al. 2010). It is reasoned that the cupin domain-containing oxalate decarboxylase from Bacillus subtilis can be functionally expressed intracellularly in honeybee commensals Fructobacillus fructosus and Lactobacillus kunkeei and that the recombinant enzyme would ultimately interact with the substrate, without the need for providing a specific ectopic transporter. The present example highlights the ample potential of enhanced honey bee commensals for the bioremediation of organic molecules in the hive.

### Materials and Methods.

### Microbiological and Molecular Techniques.

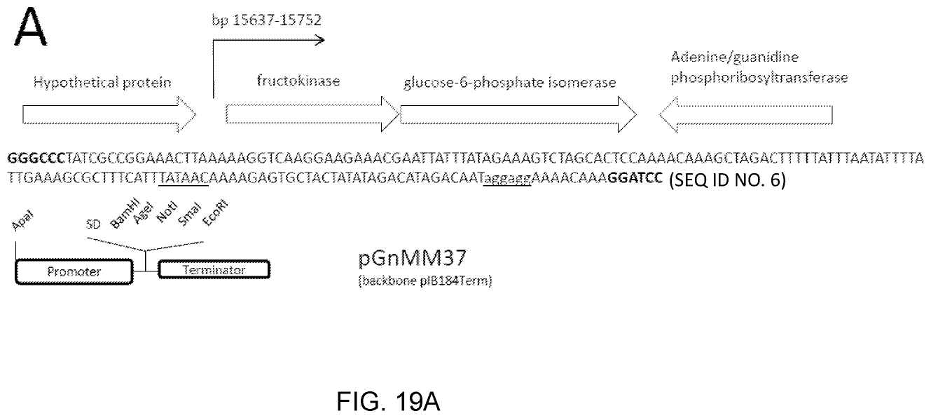

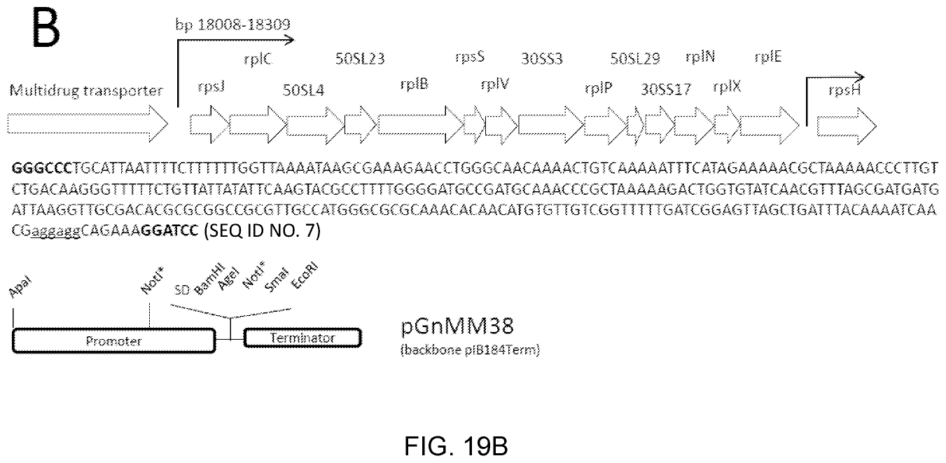

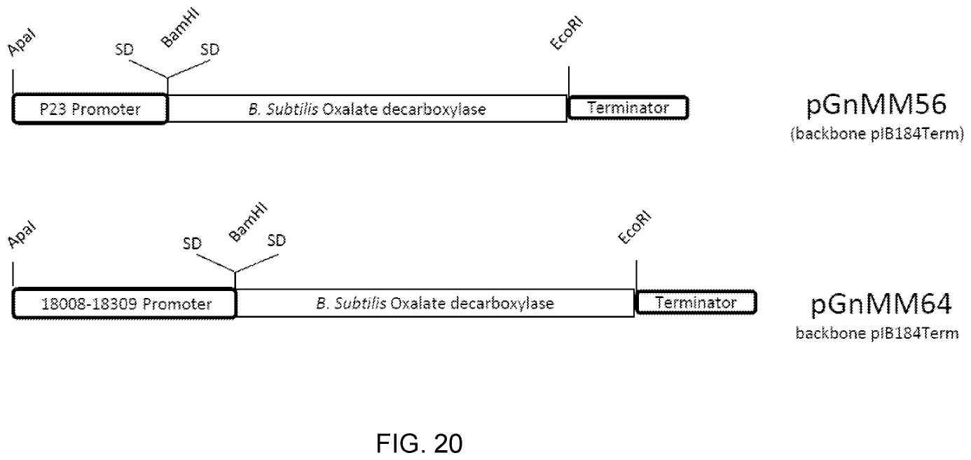

Microbiological techniques, vectors and deoxyribonucleic acid (DNA) manipulation procedure are detailed in (Maddaloni et al. 2014). Restriction enzymes, alkaline phosphatase, Phusion® Taq polymerase, and DNA ligase were purchased from New England Biolabs (NEB, Ipswich, Mass.) and used according to manufacturer's instructions. DNA sequencing was performed at the Sanger Sequencing Core Laboratory of the Interdisciplinary Center for Biotechnology Research, University of Florida. Synthetic DNA sequences were purchase from Genscript, Piscataway, N.J. To clone the oxalate decarboxylase, B. subtilis genomic DNA from a human probiotic supplement served as a template. The genomic DNA was amplified by a pair of primers, LAB258 GGATCCGTAAGGAGGAAACATTTCATG and LAB259 GAATTCGCAAGTCTTTTATTTACTGC, containing 5′ BamHI and 3′ EcoRI restriction sites. Amplification was performed for 35 cycles, extension time 20 seconds, annealing temperature 48° C. After cloning into the pMiniT vector, provided with the PCR cloning kit, the fragment was sequenced and they and 100% matched the reference sequence WP_042974741. Subsequently this insert was excised by BamHI and EcoRI restriction enzymes and inserted into expression vectors pGnMM37 and pGnMM38 (FIGS. 19A-19B; SEQ ID NOs: 6-7) generating pGnMM56 and pGnMM64, respectively (FIG. 20).

SEQ ID NO: 6
GGGCCCTATCGCCGGAAACTTAAAAAGGTCAAGGAAGAAACGAATTATTT
ATAGAAAGTCTAGCACTCCAAAACAAAGCTAGACTTTTTATTTAATATTT
TATTGAAAGCGCTTTCATTTATAACAAAAGAGTGCTACTATATAGACATA
GACAATaggaggAAAACAAAGGATCC
SEQ ID NO: 7
GGGCCCTGCATTAATTTTCTTTTTTGGTTAAAATAAGCGAAAGAACCTGG
GCAACAAAACTGTCAAAAATTTCATAGAAAAACGCTAAAAACCCTTGTCT
GACAAGGGTTTTTCTGTTATTATATTCAAGTACGCCTTTTGGGGATGCCG
ATGCAAACCCGCTAAAAAGACTGGTGTATCAACGTTTAGCGATGATGATT
AAGGTTGCGACACGCGCGGCCGCGTTGCCATGGGCGCGCAAACACAACAT
GTGTTGTCGGTTTTTGATCGGAGTTAGCTGATTTACAAAATCAACGagga
ggCAGAAAGGATCC

Transformation of Lactobacillus kunkeei and Fructobacillus fructosus

All cultures were grown static. Both F. fructosus and L. kunkeei were stocked at −80° C. in 200 μl 15% glycerol end-use aliquots and pre-cultured in 50 ml of MRS, fructose 1% overnight at 30° C. before transformation. The entire 50 ml pre-cultures were diluted in 250 ml of MRS, fructose 1%, 2 moles l⁻¹ glycine and grown at 30° C. for 4 hours. Cells were harvested and further washed 3 times with 50 ml of ice-cold distilled water and one time with 20 ml of ice-cold glycerol 10%, sucrose 0.5 moles l⁻¹. Cells were resuspended for electroporation in 0.5-0.8 ml of ice-cold glycerol 10%, sucrose 0.5 moles l⁻¹, and 70 μl aliquots were moved into 1.5 ml pre-chilled tubes, and 1-5 μg of plasmid DNA were added. After 5 min. incubation, bacterial cells were electroporated with a BTX ECM630 in 1 mm gap at 1.7 Kv, 125 Ohm, 50 μF. Cells were rested for 5 min. on ice, resuspended in 1 ml of MRS, 1% fructose, 0.5 moles l⁻¹ sucrose and recovered 3 hours at 30° C. Cells were then plated onto MRS Petri dishes containing 1% fructose containing 5 μg ml⁻¹ erythromycin and grown at 30° C. for 2-3 days, until colonies were clearly visible.

### Detection of Oxalate Degradation.

Depletion of oxalate in solid medium was detected as in (Kost et al. 2014) with minor modifications. A MRS base agar was enriched with fructose 1% and 5 mmoles l⁻¹ of MnCl₂. Bacteria were appropriately diluted and spread with a sterile loop. Petri dishes were briefly dried under laminar flow. A second layer, which contained 0.7% calcium oxalate and 0.7% agarose was freshly prepared, melted in a microwave and cooled to 47° C. in a water bath. The top agar was then quickly overlaid. Plates were incubated for at least five days before the halos became visible. Plates were then photographed using natural light and a black background. For the detection of oxalate in liquid medium, potassium oxalate was used due to its higher solubility. Bacteria were inoculated at 107 CFUs/ml in 6 ml of MRS+fructose+Mn⁺⁺ in a 14 ml tube, and incubated static at 34° C. After 7 days growth, oxalate in the supernatants was titrated with an Oxalate Assay Kit (Sigma-Aldrich).

### Results

### Construction of New Expression Vectors for Fructophilic Bacteria.

With the aim of increasing the molecular toolbox for fructophilic bacteria, the annotated F. fructosus genome (Joint Genome Institute) was analyzed. Two non-coding regions upstream the operons were arbitrarily selected hypothesizing these might act as robust promoters. The first region (FIG. 19A) is located upstream a bi-cistronic operon encoding for fructokinase and glucose-6-phosphate isomerase, whereas the second region (FIG. 19B) is located upstream a multi-cistronic operon encoding for 14 ribosomal proteins. Both putative promoters show a canonical Shine-Dalgarno (SD) sequence at the expected distance from the ATG initiation codon of the first open reading frame (ORF). Other regulatory elements, such as the −10 TATA box and the −35sequences were less evident. Both putative promoter regions were synthesized with ApaI and BamHI site at their 5′ and 3′ ends, respectively, and were subsequently cloned into pGnMM20 (Maddaloni et al. 2014), cut with ApaI and BamHI, and dephosphorylated with alkaline phosphatase, resulting in the generation of two expression vectors termed, pGnMM37 and pGnMM38 (FIGS. 1A and B). In subsequent experiments, pGnMM37 failed to accept any ORFs in the proper orientation. When pGnMM37 was used for bi-directional cloning, only the reverse orientation was accepted without rearrangements. When cloning was directionally forced, only rearranged clones were obtained and, in the case of oxalate decarboxylase, corrupted clones with a STOP codon were also retrieved. Besides oxalate decarboxylase (detailed in the next section), this situation was verified while cloning a synthetic GFP gene, as well as the Leloir operon and the β-galactosidase from Lactococcus lactis. It was concluded that the promoter borne on pGnMM37 is constitutively active in Escherichia coli, and it creates excessive disturbance to the metabolism of the cloning host. Also, experiments designed to adapt the nisin-inducible system components (Mierau and Kleerebezem 2005) from MSP3535H3 (Oddone et al. 2009) to the pIB184Term backbone generated a construct named pGnMM25 in which the oxalate decarboxylase was introduced generating a construct named pGnMM57. Despite being able to transform, pGnMM57 could not produce F. fructosus and L. kunkeei strains capable of degrading oxalate, presumably due to the toxicity of nisin and/or the lack of a nisin transporter for the induction (data not shown).

Cloning of B. subtilis Oxalate Decarboxylase.

Bacillus subtilis was isolated from a commercially available probiotic and reportedly containing 15 species of bacteria (Garden of Life, Palm Beach Gardens, USA). Dehydrated bacteria were resuspended in phosphate-saline buffer (PBS) and heated at 90° C. for 15 minutes. The heated bacterial suspension was serially diluted and plated on LB medium and incubated at 37° C. As expected, the heat treatment killed all bacteria except B. subtilis spores. In fact, the colonies retrieved on LB looked morphologically homogeneous displaying the typical swarming habit of B. subtilis. One colony was chosen and confirmed as B. subtilis, by ribosomal DNA genotypying. B subtilis genomic DNA was purified and used as template for PCR amplification of oxalate decarboxylase using the primers Fwd LAB258 and Rev LAB259. The amplified fragment migrated at the expected MW and it was cloned into pMiniT (NEB). The sequence was confirmed to be 100% identical to the original, including the Shine-Dalgarno (SD) sequence. This strategy generates expression vectors with two Shine-Dalgarno sequences upstream the ATG initiation codon. The BamHI-EcoRI insert was excised, purified, and cloned into pGNMM15 (Maddaloni et al. 2014) a construct based on pIB184Term devoid of GFP. The resulting construct, named pGnMM56 (FIG. 20), overexpresses the B. subtilis oxalate decarboxylase under the control of the constitutive p23 promoter. In parallel the same BamHIEcoRI containing the B. subtilis oxalate decarboxylase gene was cloned into pGnMM37 and pGnMM38. Cloning of the BamHI-EcoRI oxalate decarboxylase DNA fragment into pGnMM37 consistently yielded an extremely low efficiency of transformation and the majority of the clones tested were noticeably re-arranged. Two colonies appeared positive at the restriction analysis, but DNA sequencing revealed a CAG to TAG substitution which resulted in a Gln codon mutated to a stop codon early in the ORF. Cloning of the same preparation of the BamHI-EcoRI oxalate decarboxylase DNA fragment into pGnMM38 generated pGnMM64, which tested true in restriction analysis and DNA sequencing (FIG. 20).

### Degradation of Oxalates.

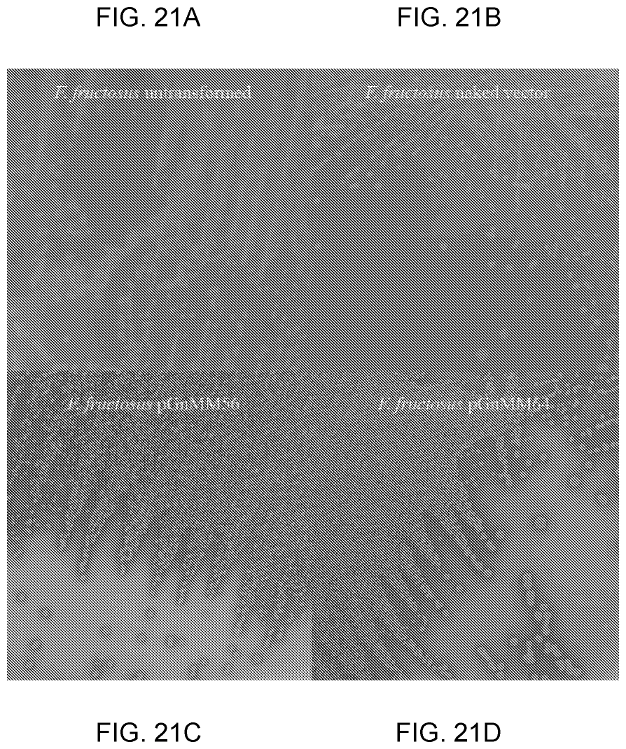

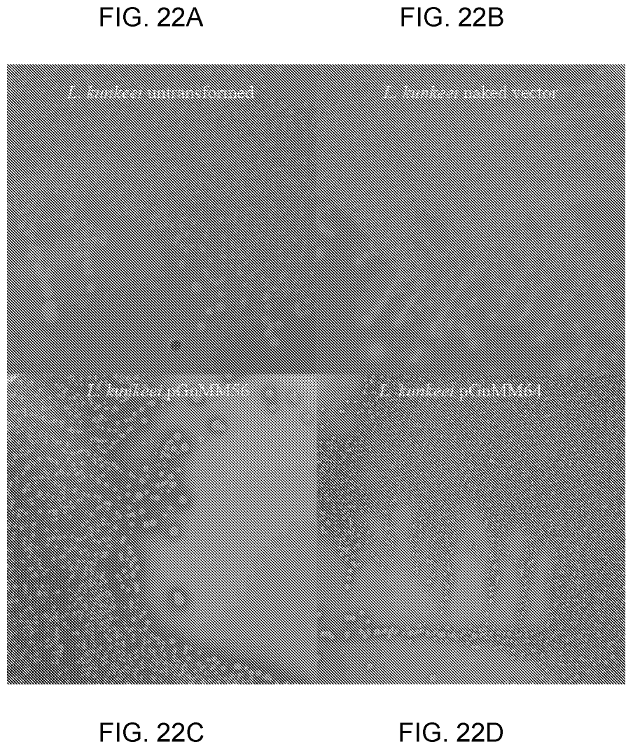

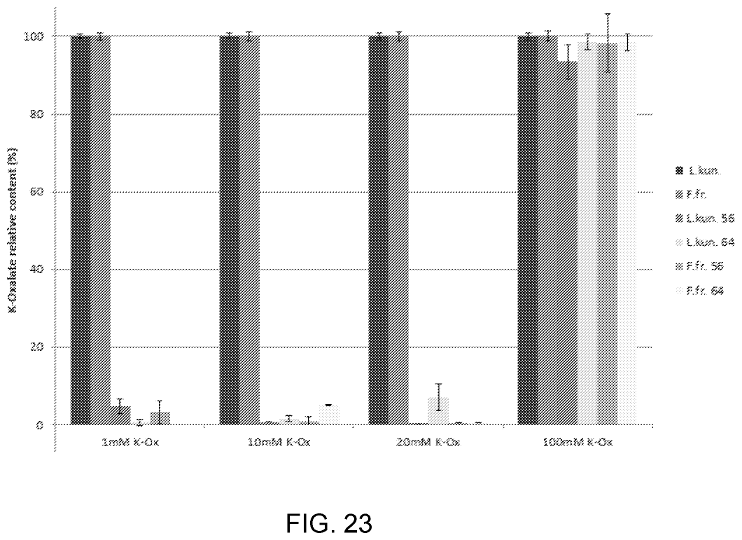

The constructs pGnMM56 and pGnMM64 were introduced into both F. fructosus and L. kunkeei with similar efficiency of transformation. Expression was evaluated both by calcium oxalate depletion halos in Petri dishes (Allison et al 1985, Kost et al 2014) and by quantitative potassium oxalate bioremediation in culture supernatant. Wild type F. fructosus and L. kunkeei, cannot degrade calcium oxalate in Petri dishes, whereas their engineered counterparts produced clear halos. FIGS. 214A-21D show that F. fructosus, native or transformed with the naked vector cannot produce a depletion halo (negative controls, top left FIG. 21A and top right FIG. 21B, respectively). Conversely, F. fructosus transformed with pGnMM56 (bottom left, FIG. 21C) or pGnMM64 (bottom right, FIG. 21D) produce clear halos of depletion of calcium oxalate. Likewise, FIGS. 22A-22D show that L. kunkeei, native or transformed with the naked vector cannot produce a depletion halo (negative controls, top left FIG. 22A and top right FIG. 22B, respectively), whereas L. kunkeei transformed with pGnMM56 (bottom left, FIG. 22C) or pGnMM64 (bottom right, FIG. 22D) produce clear halos of depletion of calcium oxalate. While only one clone per construct, either pGnMM56 or pGnMM64, is shown in each picture, four clones of F. fructosus and four clones of L. kunkeei per each construct were actually tested: all showed the halos of depletion of calcium oxalate. An interesting observation is that, when the colonies grew on the surface of the top agar, as opposed to the interface between the two layers, they did not produce halos. This behavior was consistently observed, and it can be explained by considering that these colonies become depleted of Mn⁺⁺, required for enzymatic activity (Tanner et al. 2001), which is added to the lower layer of medium, but it becomes chelated by the oxalate in the top agar. Over time, the halos of oxalate depletion grew in size and eventually became confluent. A quantitative test performed by growing the same bacteria in liquid medium confirmed the capability of the engineered strains to degrade oxalate. In this case potassium oxalate, instead of calcium oxalate, was used because of its substantially higher solubility. Increasing amounts of potassium oxalate, 1, 10, 20, 100 mmoles were added to the medium. These concentrations were purposely chosen on the ground of consideration which will be better analyzed in the Discussion section. Bacteria were inoculated at 106 CFUs/ml and grown in MRS, fructose for 7 days. Subsequently, the supernatants were titrated with an oxalate kit assay (Sigma-Aldrich) which yields linear results in the range 20 μmoles l⁻¹ up to 1.5 mmoles l⁻¹. Results are reported in FIG. 23 where the titers of the supernatants from non-recombinant controls, grown at each concentration, were plotted as 100%. Each bar is derived from two independent titrations and the whole experiment was repeated twice with virtually identical results. All recombinant strains can abate the potassium oxalate content by 95-100% up to the 20 mmoles l⁻¹ test, which is a concentration approximately 13-fold higher than the linear range of the oxalate detection kit. At potassium oxalate concentration of 100 mmoles l⁻¹ no abatement of potassium oxalate can be observed, however, at that concentration bacterial growth is severely inhibited.

### Discussion

With the rapid and hotly debated global decline of honey bee colonies, it is becoming increasingly evident that translational research, aimed at providing beekeepers with tools to improve colony health, needs to become a priority. There is a substantial hiatus between researchers and beekeepers with the latter at times, being ahead of sanctioned science, a statement that can be verified by matching the existing scientific literature and the beekeepers' chat in online fora all around the world. As an example, studies on optimization of cattle, horse and poultry nutrition initiated well over a hundred years ago. Conversely, honey bees have been, and still are, the only livestock that is kept for extended periods of times on an artificial diet composed of 100% sucrose. The practice of feeding sucrose is adopted to help bees during dearth and to build new comb. It also allows beekeepers to skim more honey. Only recently has honeybees' nutrition come under scrutiny of science and, according with what beekeepers discovered decades ago, poor diets correlated to diminished immune competence and increased overall hive weakness (Alaux et al. 2010; DeGrandi-Hoffman et al. 2010; DiPasquale et al. 2013; Morais et al. 2013). More poignant to the present investigation is the research on oxalic acid which received an inexplicable low attention by the scientific community despite the paramount relevance that this compound currently has in managing honey bee health.

The hematophagous Varroa destructor (varroa) mites are commonly believed to be a major factor driving colony losses by feeding on hemolymph, thus weakening the bees, and ferrying pathogens (reviewed in Rosenkranz et al. 2009). Despite the current relevance of oxalic acid in the fight against varroa, and the fact that first scientific reports on its use date back to the mid 90's (Radetzki 1994; Nanetti et al. 1995; reviewed in Gregorc and Planinc 2002) the recent scientific literature shows only a handful of investigations on this topic. Oxalic acid as a miticide is not uniformly regulated. However, it is safe to say that, legally or brevi manu, it is being used worldwide on a regular basis. While oxalic acid is acutely toxic to varroa, it is also toxic to honey bees (Aliano et al. 2006; Aliano et al. 2009) and it is recommended that no more than one treatment per generation of honeybees, averaging 4-6 weeks during the active season, is applied. Due to their extended lifespan, honeybee queens receive multiple treatments, a factor that may contribute to their overall decreased overall longevity. In this scenario, the capability of fine tuning the fate of oxalic acid in the hive may lead to interesting applications. Oxalic acid is a highly oxidized organic compound that is toxic to almost all organisms and cells. Oxalate anions are major chelators of metal cations, and calcium oxalate has received a great deal of attention in the medical field because it is one of the leading causes in the formation of kidney stones (Cochat and Rumsby 2013). The ability of microflora to metabolize and scavenge oxalates form the body is believed to be relevant in preventing the formation of kidney stones. In this respect, the idea of harnessing the commensal microflora, be it naturally capable of degrading oxalate or engineered, has received much attention over the last 30 years (Allison et al. 1985; Lung et al. 1991; Duong et al. 2010; Anbazhagan et al. 2013). Recently, an aggressive treatment with two weeks of daily doses of 5×1010 Lactobacillus plantarum cells, engineered with oxalate decarboxylase, proved effective in the reduction of oxalate in the kidney tissues in a rat model (Sasikumar 2014).

As a tool for fine-tuning the persistence of oxalic acid in the control of the varroa mites, oxalate decarboxylases (EC 4.1.1.2) are uniquely posed for the task, because it oxidizes both oxalates and oxalic acid generating formate and CO₂ (reviewed in Makela et al. 2010) which are both volatile and do not leave residues in the comb (Calderone 2010). In the process, energy is stored because formate can be potentially further oxidized with concomitant reduction of NAD to NADH, which is a substrate for anabolic reactions, thus decreasing the metabolic burden to engineered bacteria. Here we engineered two honey bee commensals, F. fructosus and L. kunkeei, to express a gene encoding for an oxalate decarboxylase cloned from a strain of B. subtilis that we isolated from a commercially available probiotic, advertised to be effective against kidney stones. Because oxalate decarboxylase can catabolize both oxalic acid and oxalates, calcium oxalate (in qualitative Petri dish test) and potassium oxalate (in a quantitative liquid culture test) were used in lieu of oxalic acid, under the constraint to minimize the impact of low pH on the bacterial growth. This approach, besides determining the bioremediation capability of the bacteria, is also functional in assessing the potential need to provide an oxalate transporter (Anantharam et al 1989; lyalomhe et al. 2014). As it can be seen from FIGS. 22A-22D, FIGS. 22A-22D, and FIG. 23, the engineered strains are capable of remarkable degradation of calcium oxalate. Interestingly the halo of degradation in Petri dishes did not increase in a linear manner over time, but it accelerated toward the end of the growth when the medium is naturally acidified by the production of lactic acid. This observation is in agreement with data showing that acidic pH improves enzymatic activity (Lee et. al 2014). The current configuration of the present molecular device, where the oxalic acid degrading capability is borne on an episomal genetic element, can be improved because of concerns that this feature can eventually be horizontally transmitted to other microorganisms.

Paratransgenesis, a “Trojan horse” approach originally designed to control insect-borne diseases, is being re-thought in broader terms to improve the health of beneficial arthropods, particularly honey bees (Rangberg et al. 2012, Maddaloni et al. 2014, Rangberg et al. 2015). Extensive applications of this technology can be envisioned. For example the expression of enzymes, such as some carboxylesterases capable of degrading extremely toxic insecticides, is expected to mitigate their negative effects. The present example focuses on oxalate, a molecule which, while having a moderate toxicity to honey bees, is used as a medication against the devastating varroa mites. The data presented here show that engineered honey bee commensals have the potential to remove organic molecules, oxalates in this case, in solutions at concentrations in the mg/ml range. Such a range of concentrations exceeds by several orders of magnitude the insecticide LD₅₀ that a single honey bee can absorb even from highly contaminated food. This can be projected by combining any of the honey bee LD₅₀ (ng- to μg/bee) (Sanchez-Bayo and Goka 2014) for a wide panel of insecticides, the full capacity of the honey stomach of individual honey bees (40 μl/bee) (Nicolson et al. 2013) and the expected number of bacteria contained in the whole honey bee intestine, including the honey stomach, which varies greatly depending on the study (Olofsson and Vasquez 2008, Martinson et al 2012, Corby-Harris et al. 2014). In line with this expectation the expression of a Lucilia cuprina carboxylesterase (Jackson et al 2014) in honey bee commensals can confer honey bees protection against a 40 LD₅₀ of cypermethrin (Maddaloni et al. in preparation). In conclusion the present example provides for the bioremediation of problematic organic compounds in the honey bee hive.

### REFERENCES

Alaux C, Ducloz F, Crauser D, LeConte Y. Diet effects on honeybee immunocompetence. Biol Lett 2010; 6:562-565.
Aliano N P, Ellis M D, Siegfried, B. D. (2006) Acute contact toxicity of oxalic acid to Varroa destructor (Acari: Varroidae) and their Apis mellifera (Hymenoptera: Apidae) hosts in laboratory bioassays. J Econ Entomol 2006; 99:1579-82.
Aliano N P, Ellis M D. Oxalic acid: a prospective tool for reducing Varroa mite populations in package bees. Exp Appl Acarol 2009; 48:303-9.
Allison M J, Dawson K A, Mayberry W R, Foss J G. Oxalobacter formigenes gen. nov., sp. nov.: oxalate-degrading anaerobes that inhabit the gastrointestinal tract. Arch Microbiol 1985; 141:1-7.
Anantharam V, Allison M J, Maloney P C. Oxalate:formate exchange. The basis for energy coupling in Oxalobacter. J Biol Chem 1989; 264:7244-50.
Anbazhagan K, Sasikumar P, Gomathi S, Priya H P, Selvam G S. In vitro degradation of oxalate by recombinant Lactobacillus plantarum expressing heterologous oxalate decarboxylase. J Appl Microbiol 2013; 115:880-7.
Biswas I, Jha J K, Fromm N. Shuttle expression plasmids for genetic studies in Streptococcus mutans. Microbiology 2008; 154:2275-82.
Calderone, N W. Evaluation of Mite-Away-II for fall control of Varroa destructor (Acari: Varroidae) in colonies of the honey bee Apis mellifera (Hymenoptera: Apidae) in the northeastern USA. Exp Appl Acarol 2010; 50:123-32. Cochat P, Rumsby G, Primary hyperoxaluria. N Engl J Med 2013; 369, 649-58.
Corby-Harris V, Maes P, Anderson K E. The bacterial communities associated with honey bee (Apis mellifera) foragers. PLoS One. 2014 Apr. 16; 9(4):e95056.
Damiani N, Fernandez N J, Porrini M P, Gende L B, Álvarez E, Buffa F, Brasesco C, Maggi M. D, Marcangeli J A, Eguaras M J. Laurel leaf extracts for honeybee pest and disease management: antimicrobial, microsporicidal, and acaricidal activity. Parasitol Res 2014; 113: 701-9.
DeGrandi-Hoffman G, Chen Y, Huang E, Huang M H. The effect of diet on protein concentration, hypopharyngeal gland development and virus load in worker honey bees (Apis mellifera L.). J Insect Physiol 2010; 56: 1184-91.
Degrandi-Hoffman G, Ahumada F, Probasco G, Schantz L. The effects of beta acids from hops (Humulus lupulus) on mortality of Varroa destructor (Acari: Varroidae). Exp Appl Acarol 2012; 58:407-21.
De Guzman L I, Rinderer T E, Bigalk M, Tubbs H, Bernard S J. Russian honey bee (Hymenoptera: Apidae) colonies: Acarapis woodi (Acari: Tarsonemidae) infestations and overwintering survival. J Econ Entomol 2005; 98:1796-801.
Diaz T, Del-Val E, Ayala R, Larsen J. Alterations in honey bee gut microorganisms caused by Nosema spp. and pest control methods. Pest Manag Sci. 2019; 75(3):835-843.
Di Pasquale G, Salignon M, Le Conte Y, Belzunces L P, Decourtye A, Kretzschmar A, Suchail S, Brune, J L, Alaux C. (2013) Influence of pollen nutrition on honey bee health: do pollen quality and diversity matter? PLoS One 2013; 8:e72016. doi: 10.1371/journal.pone.0072016.
Duong T, Miller M J, Barrangou R, Azcarate-Peril M A., Klaenhammer T R. Construction of vectors for inducible and constitutive gene expression in Lactobacillus. Microb Biotechnol 2011; 4:357-67.
European Parliament. On honeybee health and the challenges of the beekeeping sector. http://www.europarl.europa.eu/sides/getDoc.do?type=REPORT&reference=A7-2011-0359&language=EN. (22 Feb. 2019, last date accessed)
Gioenazzo P, Dubreuil P. Evaluation of spring organic treatments against Varroa destructor (Acari: Varroidae) in honey bee Apis mellifera (Hymenoptera: Apidae) colonies in eastern Canada. Exp Appl Acarol 2011; 55:65-76.
Gregorc A, Planinc I. The control of Varroa destructor using oxalic acid. Vet J 2002; 163:306-10.
Goulson D, Nicholls E, Botías C, Rotheray E L. Bee declines driven by combined stress from parasites, pesticides, and lack of flowers. Science 2015; 347:1255957. doi: 10.1126/science.1255957 lyalomhe O, Khantwal C M, Kang D C. The Structure and Function of OxIT, the Oxalate Transporter of Oxalobacter formigenes. J Membr Biol 2015; 248(4):641-50.
Jackson C J, Liu J W, Carr P D, Younus F, Coppin C, Meirelles T, Lethier M, Pandey G, Ollis D L, Russell R J, Weik M, Oakeshott J G. Structure and function of an insect α-carboxylesterase (αEsterase7) associated with insecticide resistance. Proc Natl Acad Sci USA. 2013 Jun. 18; 110(25):10177-82.
Kanga L H, Adamczyk J, Marshall K, Cox R J. Monitoring for resistance to organophosphorus and pyrethroid insecticides in Varroa mite populations. Econ Entomol 2010; 103:1797-802.
Klepsch A, Maul V, Koeniger N, Wachendörfer G. Einsatz von Milchsäure im Sprühverfahren zur Bekämpfung der Varroatose. Die Biene 1984; 120:199-202 & 261-262.
Kost T, Stopnisek N, Agnoli K, Eberl L, Weisskopf L. Oxalotrophy, a widespread trait of plantassociated Burkholderia species, is involved in successful root colonization of lupin and maize by Burkholderia phytofirmans. Front Microbiol 2014 4:421. doi: 10.3389/fmicb.2013.00421.
Kwong W K, Mancenido A L, Moran N A. Genome Sequences of Lactobacillus sp. Strains wkB8 and wkB10, Members of the Firm-5 Clade, from Honey Bee Guts. Genome Announc 2014; 13, 2. doi: 10.1128/genomeA.01176-14.
Lachica R V. Same-day identification scheme for colonies of Listeria monocytogenes. Appl Environ Microbiol 1990; 56:1166-1168.
Lattorff H M, Buchholz J, Fries I, Moritz R F. A selective sweep in a Varroa destructor resistant honeybee (Apis mellifera) population. Infect Genet Evol 2015; 31:169-176.
Lee E, Jeong B C, Park Y H, Kim H H. Expression of the gene encoding oxalate decarboxylase from Bacillus subtilis and characterization of the recombinant enzyme. BMC Res Notes 2014; 7:598. doi: 10.1186/1756-0500-7-598.
Lung H Y, Cornelius J G, Peck A B. Cloning and expression of the oxalyl-CoA decarboxylase gene from the bacterium, Oxalobacter formigenes: prospects for gene therapy to control Caoxalate kidney stone formation. Am J Kidney Dis 1991; 17:381-5.
Maddaloni M, Hoffman C, Pascual D W. Paratransgenesis feasibility in the honeybee (Apis mellifera) using Fructobacillus fructosus commensal. J Appl Microbiol 2014; 117:1572-84.
Maddaloni M, Pascual D W. Isolation of oxalotrophic bacteria associated with Varroa destructor mites. Lett Appl Microbiol 2015; 61(5):411-7.
Mäkelä MR, Hildén K, Lundell T K. Oxalate decarboxylase: biotechnological update and prevalence of the enzyme in filamentous fungi. Appl Microbiol Biotechnol 2010; 87:801-14.
Martinson V G, Moy J, Moran N A. Establishment of characteristic gut bacteria during development of the honeybee worker. Appl Environ Microbiol. 2012 April; 78(8):2830-40.
Mierau I, Kleerebezem M. 10 years of the nisin-controlled gene expression system (NICE) in Lactococcus lactis. Appl Microbiol Biotechnol 2005; 68:705-17.
Morais M M, Turcatto A P, Pereira R A, Franco T M, Guidugli-Lazzarini K R, Gonçalves L S, de Almeida J M, Ellis J D, De Jong D. Protein levels and colony development of Africanized and European honey bees fed natural and artificial diets. Genet Mol Res 2013; 12:6915-22.
Moritz R F. Selection for varroatosis resistance in honeybees. Parasitol Today 2014; 10:236-8.
Nanetti A, Massi A, Mutinelli F, Cremasco S. L'acido ossalico nel controllo della varroasi: note preliminari. Apitalia 1995; 22, 29-32.
Nicolson S W, de Veer L, Köhler A, Pirk C W. Honeybees prefer warmer nectar and less viscous nectar, regardless of sugar concentration. Proc Biol Sci. 2013 Jul. 31; 280(1767):20131597
Oddone G M, Mills D A. Block D E. Incorporation of nisl-mediated nisin immunity improves vector-based nisin-controlled gene expression in lactic acid bacteria. Plasmid 2009; 61:151-8.
Olofsson T C, Vásquez A. Detection and identification of a novel lactic acid bacterial flora within the honey stomach of the honeybee Apis mellifera. Curr Microbiol. 2008 October; 57(4):356-63
Radetzki T. OxalsaÈure eine weitere organische SaÈure zur VarroabekaÈmpfung. Allgemeine Deutsche Imkerzeitung 1994; 12:11-15.
Rangberg A, Diep D B, Rudi K, Amdam G V. Paratransgenesis: an approach to improve colony health and molecular insight in honey bees (Apis mellifera)? Integr Comp Biol 2012; 52:89-99.
Rangberg A, Mathiesen G, Amdam G V, Diep D B. The paratransgenic potential of Lactobacillus kunkeei in the honey bee Apis mellifera. Benef Microbes 2015; 21:1-11.
Rinderer T E, Danka R G, Johnson S, Bourgeois A L, Frake A M, Villa J D, De Guzman L I, Harris J W. Functionality of Varroa-resistant honey bees (Hymenoptera: Apidae) when used for western U.S. honey production and almond pollination. J Econ Entomol 2014; 107:523-30.
Robijn S, Hoppe B, Vervaet B A, D'Haese P C, Verhulst A. Hyperoxaluria: a gut-kidney axis? Kidney Int 2011; 80:1146-58.
Rosenkranz P, Aumeier P, Ziegelmann B. Biology and control of Varroa destructor. J Invertebr Pathol 2010; 103 Suppl 1:S96-119.
Sabahi Q, Gashout H, Kelly P G, Guzman-Novoa E. Continuous release of oregano oil effectively and safely controls Varroa destructor infestations in honey bee colonies in a northern climate. Exp Appl Acarol 2017; 72(3):263-275.
Sanchez-Bayo F, Goka K. Pesticide residues and bees—a risk assessment. PLoS One. 2014 Apr. 9; 9(4):e94482.
Sasikumar P, Gomathi S, Anbazhagan K, Abhishek A, Paul E, Vasudevan V, Sasikumar S, Selvam G S. Recombinant Lactobacillus plantarum expressing and secreting heterologousoxalate decarboxylase prevents renal calcium oxalate stone deposition in experimental rats. J Biomed Sci 2014; 30:21:86. doi: 10.1186/s12929-014-0086-y
Tanner A, Bowater L, Fairhurst S A, Bornemann S. Oxalate decarboxylase requires manganese and dioxygen for activity. Overexpression and characterization of Bacillus subtilis YvrK and YoaN. J Biol Chem 2001; 276:43627-34.
The Bee Informed Partnership. Honey Bee Colony Losses in the U.S. Winter 2014-2015. https://articles.extension.org/pages/58013/honey-bee-winter-loss-survey. (22 Feb. 2019, last date accessed).
Umpiérrez M L, Santos E, Mendoza Y, Altesor P, Rossini C. Essential oil from Eupatorium buniifolium leaves as potential varroacide. Parasitol Res 2013; 112:3389-400.
USDA. ARS Honey Bee Health and C.C.D. https://www.ars.usda.gov/oc/br/ccd/index. (22 Feb. 2019, last date accessed).

### Example 8

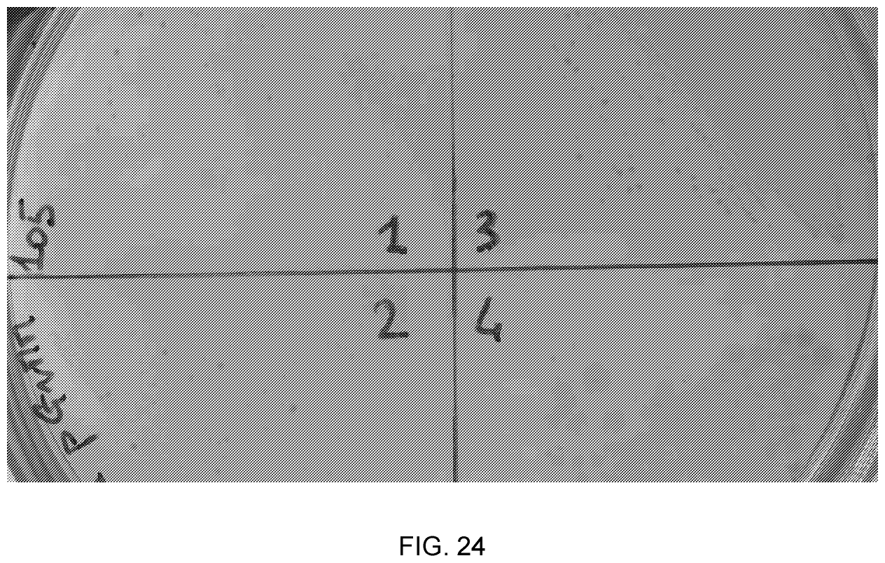

FIG. 24 Pseudomonas putida KT2440 engineered with a construct bearing the cassette borne on pGnMM83 (FIG. 17), which confers the capability to use 6-chloronicotinic acid as a sole carbon source, provided that a nicotinic acid degradation pathway (FIG. 14) is functional. Four clones are shown.

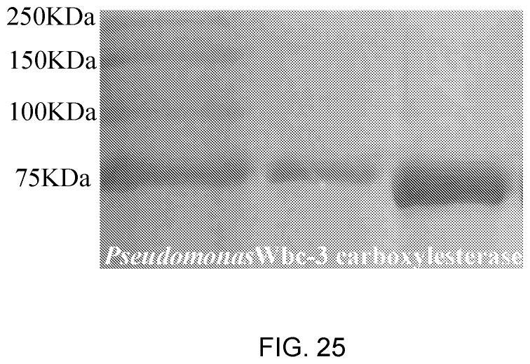

FIG. 25 shows expression of Pseudomonas Wbc-3 carboxylesterase.

### Example 9

Additional examples of oxalate decarboxylases, with sequences, are described below:

SEQ ID NO 8: Burkholderia pseudomallei Oxalate decarboxylase protein
MTNLSRRKMLAGTAGALAAAGIAVSAKAASFGNPDSPAEGAVNA
RNPQSLTDPGPQNPALMKEFPSFQSPPATDINGMPIFWASFNNAHKRIQNGGWAREVT
QEDFAISETISGVNMRLARGGIREMHWHQQAEWAFMLDGRCRITVLDEEGRPSVQDVK
TGDLVVYFPPGLPHSLQGLGVDGAEFLLAFDNGRASEFNTLLVTDWIAHTPPDVLALNF
GVPADAFRRIPLDNLWIFQGDDPGPLAAAQRASASSRGAPKHPFIFSMGDMKPNVKTR
GGEVRIVDSTNFAVSKTIAAALVTVKPGGMRELHWHPNADEWQYYIRGDARMTVFDTG
PKAQTADFRAGDVGYVKKSLGHYVQNTGTTDLVFLEIFKADRYAEVSLSDWLAHTPPQ
LVEAHLHIAPDVIARFPRNRPDVVPA
SEQ ID NO 9: Burkholderia pseudomallei Oxalate decarboxylase nucleic acid
atgacgaatctgtcccgacgcaagatgctagccggcacggccggcgcgctcgccgccgccggcatcgccgtttccgcgaa
ggccgcttcgttcggcaatccggacagcccggccgagggcgcggtgaacgcgcgcaatccgcaaagcctgaccgatccgg
ggccgcaaaatccggcgctgatgaaggagtttccttcgtttcagagtccgccggccaccgacattaacggcatgccgatt
ttctgggcgtcgttcaataatgcgcacaagcgcattcagaacggcggctgggcgcgcgaagtcacgcaggaggatttcgcgatt
tccgaaacgatttccggcgtcaacatgcggctcgcgcgcggcggcattcgcgagatgcactggcaccagcaggccgaatggg
cgttcatgctcgacggccgctgccggatcaccgtgctcgatgaagaggggcggccgtccgtgcaggacgtgaagacgggcga
cctctggtatttcccgccggggctgccgcattcgctgcaggggctcggcgtcgacggcgccgaattcctgctcgcgttcgac
aacggccgcgcgtccgaattcaacacgctgctcgtgacggactggatcgcgcacacgccgcccgacgtgctcgcgctgaa
cttcggcgtgcccgccgatgcattccgccgcattccgctcgacaatctgtggatcttccagggcgacgatcccgggccgc
tcgccgccgcgcagcgcgcgtcggcgtcgtcgcgcggcgcgccgaagcatccgttcatcttctcgatgggcgacatgaag
ccgaacgtgaagacgcgcggcggcgaagtgcggatcgtcgacagcacgaacttcgctgtgtcgaagacgatcgcggccgc
gctcgtcacggtgaagccgggcggcatgcgcgagctgcactggcacccgaacgcggacgagtggcagtactacatccgggg
cgacgcgcgcatgacggtgttcgacaccggcccgaaggcgcagacggccgatttccgcgcgggcgacgtcggctacgtg
aagaagagcctcggccactacgtgcagaacacgggcacgaccgatctcgtgttcctcgagatcttcaaggcggaccgcta
cgcggaggtttcgctgtccgactggctcgcgcacacgccgccgcagctcgtcgaggcgcatctgcatatcgcgcccgacg
tgatcgcgcgctttccgcgcaaccggccggacgtcgtgccggcgtaa
SEQ ID NO 10: Pantoea allii oxalate decarboxylase protein
MSKLTRRSFMAFAAGGTVALAAGQVHAHDSIQSFPTGGRDPGPH
DPIREAENPDIVNPPSTDSGTLPNLRFSFSDAHVRKSSGGWTRQVTKRELAISTTIAG
VDMRLNAGGIRELHWHKEGEWAYMTYGNARVTAFDTDGSWFIDDIGPGDLWYFPPGIP
HSIQGIGPDGCEFLLAFDDGGFDEDSTFLLSDWFKHIPPDILAKNFNVPVSTFDHLPS
PADEYIFAAKVPGSLESVAVKGATKAKRTFTHHMLAQEPIKAPGGTVRITDSSTFKVS
KTIAAALVEIAPGGLRELHWHPNNDEWQYYLEGEGRMGVFASSGQARTFDYRAGDVGY
VPFAMGHYVENTGKGPLRFLELFKSDYYADISLNQWLAGTPRELVKAHLQLDDAFMDA
LSLQKNPVVK
SEQ ID NO 11: Pantoea allii oxalate decarboxylase nucleic acid
atgagtaaattaacccgaagaagttttatggcgtttgccgcaggcgggacggtggcgctggcggctggacaggttcatgc
gcatgacagcattcagtcctttccgaccggtggtcgcgatcccgggccgcacgatcccatccgcgaagcagaaaatccgg
acattgttaatccaccttcgaccgacagcgggacgttaccgaacctgcgtttttcattcagtgatgcgcacgtgcgcaaa
agcagcggcggatggacgcgccaggtcaccaagcgtgaattagccatttcgaccaccattgccggtgtggacatgcgcct
gaatgccggcggcatccgcgaattacactggcataaagagggcgaatgggcctacatgacctatggcaacgcccgcgtca
ccgcgtttgataccgatggcagttggtttatcgatgatattggcccgggagacttatggtatttcccgccgggcatcccg
cactcaattcagggcatcgggcctgatggatgcgaatttctgctggccttcgacgatggcggatttgatgaggacagcac
gtttctgttatccgactggttcaaacatatcccgccggacattctggcgaaaaacttcaacgtcccggtatccaccttcg
accacctgccttcgccagcggatgaatatatttttgccgcgaaggtgcccggcagcctggagtcagtggcggtgaaaggc
gcaaccaaagccaaacgcacctttacccaccacatgctggcacaggaacccataaaggcaccgggcggcacggtcagaat
caccgactcctcaacctttaaggtgtccaaaactatcgcggcagccctggttgaaatcgcccccggcggattacgcgagt
tgcactggcacccgaataacgatgaatggcaatactacctggaaggggaaggacgcatgggcgtttttgcctcatcgggt
caggcaagaacatttgattatcgggcaggggatgtcggctatgtgcccttcgccatgggccactatgttgaaaacaccgg
caaggggccgctgcgtttcctggagttattcaaaagtgactattacgccgatatctcactcaatcagtggctggccggta
cgccgcgtgagctggtaaaagcgcatctccagttagatgacgcatttatggatgcgctgtcgctgcagaaaaatcctgtg
gtgaaataa
Example 10:
SEQ ID NO 12: Cassette expressing transporter and cch2 (6-CNA Chlorohydrolase; including
HindIII and BamHI cloning ends) (nucleic acid)
AAGCTTaggaggtctagaaatatgatgcgacgtgtacttgtccttggtatctttatgattactcaatctgtagttgcagaagctgca
gatgaaatcaaaattggtgaaatcaactcttattctacagcagctagttttactgaaccatatcgtaaaggttggcaacttgcagtag
atgaaatcaatgctaaaggaggtgttcttggcaagaaacttatcgttattgcaaaggacgatgctggaaaacctgctgatgcgatt
actgctgcgaatgatcttatggcaaacgaaggtgtcgttatgttagctggtacatttctttctcatgtagctctcgcagtatcagatttcg
ccaaacagaaaaagatcgtgtttatggctggtgaacctttgacagatgcacttacgtggtcacaaggtaataagtacacattccgt
cttcgcccaaatacctatatgcaagctgcaatgttggctgaagaggcagcgaaactgccagcaaaacgttgggctaccattgctc
cgaactatgagtttggacaatctgcagttgcagctttcaagaaacttttgtctcaaaaacgtccagatgttgaatgggtaggagaac
aatggccacctctcggcaagattgatgctggaccagtagttcaagcacttgctgcaactaaaccagatgcaattatgaatgcagt
atttggagttgatctcgccaaatttgtacgtgaaggtaatgcacgtgctctgttcaaagatagatcagttgtaagtttcttgacaggtga
accagagtttctcgatcctctgaaagacgattcacctgttggctggattgtgacaggttatccttggtatggaatcaaaactccagaa
catgatgcgtttctcaaagcatatcaagcaaaacataaagattatcctcgtcttggttctgtagttggctatcaacccattatggctgc
agccgctatgcttaacaaagcaggatcaactgatactgataaacttatcgcagcgggtgaagatttgaagatggaatcaccattt
ggaccaatcgtactccgtaaaattgatcatcaagctacattaggagattatatcggtaaaatcgctattaaggatggtatgggagtg
atggtagacttcaaatactttgatggagctcaatatctacctacagatgcagaaatcggaaaaatgcgtccaaaggattaaatac
gttcactaaggagggtctctagaatgagtcttattgcaatcaaaaatgcagattggattcttactatggatggagctcgtcgagttatt
gcagatggatctatcttgattgacaaagatcgtattcgagcaattggtaaatctgctgtgattgaaatccctccagaaacaactaag
gttattgatggtcgtggaaaagttgtattaccaggattgattgattctcatacacaccattcacttcatttaggtcgtggacttgcagatg
aatgtgacattcaaacgtttctgtatcgacgtttgtatcccattgaagcatcacttaatgatgaagatgcttacattagtgcattgttatgt
caacttgaaatgattaagtctggtactacatcgattatcgatgctggaaactactttcctgaagcaacgcttcgtgcatttggaacaa
ctggtatgcgtggagttgtagcacgaagtacgtttgatattccaacatcttcacttggatcattgccagcacaagtgtttgctgaagag
accaatgttgcattgaaacgtgctgaagagtttgtagaacgaaacagtggtgcttgtgatggtcgtgtgcaagcatggttacaattg
cgtgttcttccgaattgttctgatgaactttgccgtggcttaaaaagtattgctgatcgacttggagttcgttatgaagcacatgcagcct
tcactaaagaagtctatgaagcctctaaactgcagtttggtaaatctgaagttcgtcgcttggatgacctcggtatcctcggtgaagg
attattgctcgcgcatatgggatggttaacgccacgtgacatcttgttactcattagttctaaaactaatgttgttctttgtccaacatcta
gtgtacatcaagcgatgggtagcattgcatttggacatgtccctgagttgttagaaatgggagttaacgttgctcttggaacggatgg
cggtcctcatggaactaatgatcttgttcgccaaatctttgtagctgcaggaggctataaggaagttcgtcttgatgcaacaatcatg
ccacctgagacagtacttgaaatggcaaccgttaacggtgctcgtgcaatgggaatgtcagatcaagtaggatctattgaacctg
gtaagaaagcagacattacaatctttgattctcgtcgacctgaatggcgtccattgcacaatccagtcgcgaatctcgtatattgcgc
aaacggaaattctgctgataccgttattgtagatggacgtatcttgatggagaatcgtaaaattctcaccttcaacgaagatgacgt
aattactgaagctcaacgtcgatcagttgagattggtgctcgtgcaggattgttagaatatggacgaccacgctggccagtccattg
aagagatataaaaagccagattattaatccggcttttttattatttGGATCC
SEQ ID NO 13: Cassette expressing transporter and cch2 (6-CNA Chlorohydrolase)
(protein)
MMRRVLVLGIFMITQSVVAEAADEIKIGEINSYSTAASFTEPYRKGWQLAVDEINAKGGV
LGKKLIVIAKDDAGKPADAITAANDLMANEGVVMLAGTFLSHVALAVSDFAKQKKIVFMA
GEPLTDALTWSQGNKYTFRLRPNTYMQAAMLAEEAAKLPAKRWATIAPNYEFGQSAVAAF
KKLLSQKRPDVEWVGEQWPPLGKIDAGPVVQALAATKPDAIMNAVFGVDLAKFVREGNAR
ALFKDRSVVSFLTGEPEFLDPLKDDSPVGWIVTGYPVVYGIKTPEHDAFLKAYQAKHKDYP
RLGSVVGYQPIMAAAAMLNKAGSTDTDKLIAAGEDLKMESPFGPIVLRKIDHQATLGDYI
GKIAIKDGMGVMVDFKYFDGAQYLPTDAEIGKMRPKD
MSLIAIKNADWILTMDGARRVIADGSILIDKDRIRAIGKSAVIEIPPETTKVIDGRGKVV
LPGLIDSHTHHSLHLGRGLADECDIQTFLYRRLYPIEASLNDEDAYISALLCQLEMIKSG
TTSIIDAGNYFPEATLRAFGTTGMRGVVARSTFDIPTSSLGSLPAQVFAEETNVALKRAE
EFVERNSGACDGRVQAWLQLRVLPNCSDELCRGLKSIADRLGVRYEAHAAFTKEVYEASK
LQFGKSEVRRLDDLGILGEGKTNVVLCPTSSVHQAMGSIAFGHVPELLEMGVNVALGTDG
GPHGTNDLVRQIFVAAGGYKEVRLDATIMPPETVLEMATVNGARAMGMSDQVGSIEPGKK
ADITIFDSRRPEWRPLHNPVANLVYCANGNSADTVIVDGRILMENRKILTFNEDDVITEA
QRRSVEIGARAGLLEYGRPRWPVH

It is noted that the above cassette is optimized for expression in Lactococci with adaptability to E. coli and Pseudomonas putida.

### Example 11

The cch2 cassette described above in the examples, and examples of sequences of which are shown in Example 10, can be expressed in a host with an endogenous 6-HNA degradation operon, for example that of P. putida KT2440. Such expression of the cch2 cassette in an organism with an operable 6-HNA degradation operon allows for the utilization of 6-CNA as a carbon source, as neonicotinoids can be degraded into 6-CNA, and the resultant 6-CNA funneled into the 6-HNA degration operon pathway (FIG. 14), allowing for the degradation of 6-CNA into fumaric acid which can then enter the krebs cycle (FIG. 24).

Further information regarding the P. putida 6-HNA degradation operon is described in Jimenez J I, Canales A, Jimenez-Barbero J, Ginalski K, Rychlewski L, Garcia J L, Diaz E. “Deciphering the genetic determinants for aerobic nicotinic acid degradation: the nic cluster from Pseudomonas putida KT2440. Proc Natl Acad Sci USA. 2008 Aug. 12; 105(32):11329-34. doi: 10.1073/pnas.0802273105. Epub 2008 Aug. 4, which is incorporated by reference as if fully set forth herein.

Briefly, an example of a fully-functional 6-HNA degradation operon (top of FIG. 16) lies in a 13 kb region of the Pseudomonas putida KT2440 chromosome, complete genome, from base 4441000 to 4454000 (NCBI reference sequence NC_002947.4) and comprises nucleotide sequences encoding the following peptide coding sequences (of which recombinant nucleotide sequences can be derived according to methods known in the art, although some of the nucleotide sequences may be nested and overlapping):

SEQ ID NO 14: porin-like protein, nicP (NCBI accession NP_746069.1)
MQGFTAPRFSSSARLAGGCIAAALCAEATAEEAGFIEGARATLQARNYFFSRDYADIKGAST
QSRTQEWAQGFILNASSGYTQGTLGVGVDVTGLLGFKLDSSPEHARSGLLPSLDSGKSAD
EYSRLGAAIKFKVSQTELKVGELMPNLPVLLFSDLRLLPPTYQGAMLESREFAGLTLSAGQF
RSTSLRDSSNSQKMYALVNDPINPARLARFTSDRFNYVGADYAFNDNRTSVGVWQAQLED
IYQQRFYSFKHAEPLGSWTLGVNAGYFDAREDGSKVAGDYDNHALFSLFSAKTGGHTFYV
GYQQIGGDDGFIQVGANTNPMGNTLPTYEFSAPGERSWQVRHDYNFVALGLPGLTSTLRY
VKGRDVETGLGFEGRDRERDLDLAYVVQSGPLAGLGIRVRNVMARSNYRTDIDENRLILSY
TIKVF
SEQ ID NO 15: metabolite transport protein, NicT (NCBI accession
NP_746070.1)
MPIANATTVHSDIDHGTKALYSKITWRLIPFLCFCYLAAYLDRINVGFAKLQMLEDLQFSTAA
YGLGAGLFFVGYIIFEVPSNLILQRVGAKLWIARIMITWGLLSACTMFVTSTTQFYILRFLLGA
AEAGFLPGVLYYLTMWYPTYRRGRIIALFMIGLPLSSVIGGPISGWIMGHFDQVQGLHGWQ
WLFLLEAIPSVLLGILTFWALPNHFQQAKWLSADDKAQLAADLAADDAEGKDSKHSFRDGF
FNLKVWMLGGIDFSILLSAYAMGFWMPTFIRDAGVSDTFHIGLLTAIPSLAALAGMLMIGASS
DRHRERRWHIIVPFIIGAIAMASSTLFSQNLVMTVVLFAIASAAIIGAVPVFFSLPATFLKGTAA
ATGFALACSVANIAGLVSNSLMGVVTDLTGTSHAALWVFAGCLILSCFLVIALPAKLVNR
SEQ ID NO 16: maleamate amidohydrolase, NicF (NCBI accession
NP_746071.1)
MSDAQSARDNYQGVWGQRIGFGRKPALLMIDFMQGYTTPGAPLYAPGVVAAVEQAAGLL
ALARDCGTLVVHTNIRYQPPHFADGGVWVRKAPVMKDMVEGNPLAAFCEAVAPQAGEVVL
SKQYASAFFATSLAPLLHAQGVDTVVLAGCSTSGCIRASAVDAMQHGFRTIVVRECVGDRH
SDPHEANLFDIDSKYGDVVTRQDAMQQLRHLAG
SEQ ID NO 17: maleate isomerase, NicE (NCBI accession NP_746072.1)
MTQLYRIGQIVPSSNTTMETEIPAMLNARQAIRPERFTFHSSRMRMKQVKKEELAAMDAES
DRCAVELSDAKVDVLGYACLVAIMAMGLGYHRQSEKRLQQATADNDALAPVITSAGALVEA
LHVMKAKRIAIVAPYMKPLTELVVNYIREEGFEVQDWRALEIPDNLAVARHDPANLPGIVAG
MDLEGVDVVVLSACVQMQSLPAVAKVEAQTGKPVVTAAIATTYAMLKALDLEPIVPGAGALL
SGAY
SEQ ID NO 18: N-formylmaleamate deformylase, NicD (NCBI accession
NP_746073.1)
MSTFVAGGNVSANGIRQHYLRYGGKGHALILVPGITSPAITWGFVAERLGHYFDTYVLDVR
GRGLSSSGPDLDYGTDACAADIPAFAAALGLDSYHLLGHSMGARFAIRAAAQGAPGLQRLV
LVDPPVSGPGRRAYPSKLPWYVDSIRQATVGMSGDDMRAFCATWSDEQLALRAEWLHTC
YEPAIVRAFDDFHEVDIHQYLPAVRQPALLMVAGRGGVIEPRDIAEMRELKPDIQVAYVDNA
GHMIPWDDLDGFFAAFGDFLDHPLV
SEQ ID NO 19: 6-hydroxynicotinate 3-monooxygenase, NicC (NCBI
accession NP_746074.1)
MRGRQKIAIVGAGLGGAAAATLLQQAGFDVEVFEQAPAFTRLGAGIHIGPNVMKIFRRMGLE
QKLELMGSHPDFWFSRDGNTGDYLSRIPLGEFARREYGAAYITIHRGDLHALQIEAIQPGTV
HFGKRLEKIVDEGDQVRLDFADGTHTVADIVIGADGIHSKIREELLGAEAPIYSGVVVAHRALI
RGVNLAQHADVFEPCVKWWSEDRHMMVYYTTGKRDEYYFVTGVPHEAWDFQGAFVDSS
QEEMRAAFEGYHPTVQKLIDATESITKWPLRNRNPLPLWSRGRLVLLGDACHPMKPHMAQ
GACMAIEDAAMLTRCLQETGLSDHRTAFALYEANRKERASQVQSVSNANTWLYSQEDPA
WVYGYDLYGQQLESGEAA
SEQ ID NO 20: 2,5-dihydroxypyridine 5,6-dioxygenase, NicX (NCBI
accession NP_746075.1)
MPVSNAQLTQMFEHVLKLSRVDETQSVAVLKSHYSDPRTVNAAMEAAQRLKAKVYAVELP
AFNHPTAMGNDMTAYCGDTALTGNLAAQRALEAADLVVDTMMLLHSPEQEQ1LKTGTRILL
AVEPPEVLARMLPTEDDKRRVLAAETLLKQARSLHVRSKAGSDFHAPLGQYPAVTEYGYAD
EPGRWDHWPSGFLFTWPNEDSAEGTLVLDVGDIILPFKNYCRERITLEIEKGFITGIHGGFEA
EYLRDYMKYFNDPEVYGISHIGWGLQPRAQWTAMGLHDRNDGMCMDARAFYGNFLFSTG
PNTEVGGKRKTPCHLDIPLRNCDIYLDDKAVVLAGDVVAPEESRAR
SEQ ID NO 21: HTH-type transcriptional repressor, NicR (NCBI
accession NP_746076.1)
MSKKTTPSSAPLDNTPYDVTEQVGHLLRKAYQRHTAIFQQQACDPQLTSIQFVTLCALRDH
GPSSQAELIKATAVDQATIRGIVERLKARELVQLSPDPGDRRKVIVELTESGAALLDAMIPCA
RQISELSMGSLNAGERVAILYLLRKMIDSDENAG
SEQ ID NO 22: nicotinate dehydrogenase subunit A, NicA (NCBI
accession NP_746077.1)
MQTTISLQVNGQPVEVSAMPDTPLLLILRNDLCLNGPKYGCGLGECGACTVIIDGVAARSCV
IPLAGAAGRNITTLEGLGSKAAPHPVQQAFIDEQAAQCGYCMNGMIMTAKALLDRIPEPSDE
QIRNELSANLCRCGTHVEILRAVRRAAETRRKP
SEQ ID NO 23: nicotinate dehydrogenase subunit B, NicB (NCBI
accession NP_746078.1)
MNHSQQVPSRDQLLAKTGVLLIVDQITPPSGPVAKGVTPTVKERELALFIAVSDDGMVYAFN
GHVDLGTGIRTSLAQIVAEELDLRMDQVHMVLGDTERAPNQGATIASATLQISAVPLRKAAA
TARRYLLQQAALRLGCPPEMLRIEDGTVIASNGSTLSFAELVQGKNHQLHIADDAPLKAIEDY
RLVGRSAPRVDIPGKATGELTYVHDMRLPNMLHGRVIRPPYAGHDSGDFVGNSLLAVDES
SIAHLPGVVAVVVIRDFVGVVAEREEQAIRAAHELKVSWKPFTGKLPDLSDVAQAIRDNPRV
QRTVLDQGDVDGGIANASQRLSRSYLWPYQLHASIGPSCALADFTAGQIRVWSGTQNPHL
LRADLAWLLACDEARIEIIRMEAAGCYGRNCADDVCADAVLLSRAVQRPVRVQLTREQEHV
WEPKGTAQLMEIDGGLNADGSVAAYDFQTSYPSNGAPTLALLLTGAVEPVPALFEMGDRT
SIPPYDYEHMRVTINDMTPLVRASWMRGVSAMPNSFAHESYIDELAFAAGVDPVEYRLKHL
SDPRAIDLVKATAERAQWQPHTRPMQTQAEGDVLRGRGFAYARYIHSKFPGFGAAWAAW
VADVAVDRRTGEVAVTRVVIGHDAGMMVNPEGVRHQIHGNVIQSTSRVLKEQVSFEESTV
ASKEWGGYPILTFPELPAIDVMMLPRQHEPPMGSGESASVPSAAAIANAIFDATGIRFRELPI
TAERVRAALGGEGQGPDAPAPAQPSTKRSKWWFGSLAGVFGAALGMLATALPWRAEIAP
VTPPGVGSWSAAMLERGRQVAAAGDCAVCHTVSGGKANAGGLAMDTPFGTLYSTNITPD
PETGIGRWSFAAFERAMREGISRDGRHLYPAFPYTSFRNINDADMQALYAYLMSQTPVRQ
EAPANQMRFPFNQRPLMAGWNARFLQRGEYQPDPQRSAQWNRGAYLVDGLGHCTACHS
PRNLMGAEKGGSSYLAGGMVDGWEAPALNALGKSSTPWSEDELFNYLSTGFSEKHGVAA
GPMGPVVSELATLPKSDVRAIAHYLSSLEGEPQALAANAAPQVDTHVSLSNGERVFKGAC
LGCHSDGLGPKLFGVSPSMAVNSNVHSDLPDNLLRVVLHGIPTPATRDLGYMPGFKDSLSD
RQVADLAAYLRHRFAADKPAWQGLASKAAQVRANPGSH
SEQ ID NO 24: HTH-type transcriptional repressor, NicS (NCBI
accession NP_NP_746079.1)
MQTRKTGVRAQQADRTRDNILKAAVKVFSKEGFTGGRIEQISTLAKSNDRMIYYYFGSKEKL
FISVLEHIYASFNQAEAKLRLDLADPEQALRELVAFIWDYYVRHPEFVTILATENLHQGLHAR
KSQNLRALSGEAVGVLRPIIEAGQAKGLFRDDICITHAYLMIASLCYFYNSNRHTLSSFLAVD
LADKQAKADWLTFISDLALRGLRR

If not expressed endogenously, as illustrated in FIGS. 15A and 16, cassettes comprising recombinant nucleotide sequences encoding coding sequences of the peptides above can be incorporated into expression vectors for exogenous expression in a host in the exact order and direction as shown, at least regarding coding sequences of NicC, NicX, NicD, NicF, and NicE.

It is noted that recombinant codon-optimized coding sequences of gene products as listed above can be derived from the 13 kb region of the Pseudomonas putida KT2440 chromosome, complete genome, from base 4441000 to 4454000 (NCBI reference sequence NC_002947.4) using the full peptide coding sequences above and cloned into expression backbones as fully functional cassettes or individual coding sequences for one or more of the gene products above.

Unless defined otherwise, all technical and scientific terms used have the same meaning as commonly understood by one of ordinary skill in the art to which this disclosure belongs. Although any methods and materials similar or equivalent to those described can also be used in the practice or testing of the present disclosure, the preferred methods and materials are now described.

Embodiments of the present disclosure will employ, unless otherwise indicated, techniques of separating, testing, and constructing materials, which are within the skill of the art. Such techniques are explained fully in the literature.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

1) An engineered cell, comprising:
a bacterium containing therein one or more stably-expressing expression vectors for exogenous expression of one or more recombinant carboxylesterase enzymes, thereby providing the engineered cell an exogenous pathway for hydrolyzing ester bonds,
one or more recombinant oxalate decarboxylase enzymes, thereby providing the engineered cell an exogenous pathway for removing carboxyl groups,
one or more recombinant 6-chloronicotinic acid degradation enzymes, thereby providing the engineered cell an exogenous pathway for degrading 6-chloronicotinic acid, or
a 6-HNA degradation operon, individually or in combination.

2) The engineered cell of claim 1, wherein the bacterium is a bacteria of the genus Escherichia, Lactobacillus, Fructobacillus, Gilliamella, Snodgrassella, Actinobacteria, Parasaccharibacter. Pseudomonas, Stenotrophomonas, Sphigobacterium, or Bacillus.

3) The engineered cell of claim 1, wherein the one or more stably-expressing vectors comprise one or more coding sequences for a recombinant carboxylesterase,
one or more coding sequences for a recombinant oxalate decarboxylase,
one or more coding sequences for a recombinant 6-chloronicotinic acid chlorohydrolase (cch2), or
one or more coding sequences encoding one or more recombinant gene products of a 6-HNA degradation operon, individually or in combination.

4) The engineered cell of claim 1, wherein the one or more stably-expressing vectors comprise one or more coding sequences with about 90% to about 100% sequence identify with SEQ ID NOs: 1-5, SEQ ID NOs: 6-11, SEQ ID NOs: 12-13, or SEQ ID NOs: 14-24.

5) The engineered cell of claim 3, wherein the recombinant carboxylesterase enzyme is Methyl Parathion Hydrolase (MPH), Pyrethroid Hydrolase (PytH), E3 carboxylesterase, E3^{Trp251Leu} carboxylesterase, Deinococcus radiodurans phosphotriesterase, or Sulpholobus acidocaldarius phosphotriesterase;
wherein the oxalate decarboxylase is a B. subtilis, Burkholderia pseudomallei, or Pantoea allii oxalate decarboxylase; and
wherein the recombinant 6-chloronicotinic acid chlorohydrolase is a Bradyrhizobiaceae bacterium cch2.

6) A modified bee bread, comprising:
bee bread; and
one or more purified recombinant carboxylesterases, one or more purified recombinant oxalate decarboxylases, one or more purified recombinant 6-chloronicotinic acid chlorohydrolases (cch2), one or more recombinant gene products of a 6-HNA degradation operon, one or more engineered cells, or a combination thereof.

7) The modified bee bread of claim 6, wherein the bee bread is wild-type bee bread.

8) The modified bee bread of claim 6, wherein the bee bread is synthetic bee bread.

9) The modified bee bread of claim 6, wherein the one or more purified recombinant carboxlyesterases are Methyl Parathion Hydrolase (MPH), Pyrethroid Hydrolase (PytH), E3 carboxylesterase, E3^{Trp251Leu} carboxylesterase, Deinococcus radiodurans phosphotriesterase, or Sulpholobus acidocaldarius phosphotriesterase;
wherein the one or more purified recombinant oxalate decarboxylases are a B. subtilis, Burkholderia pseudomallei, or Pantoea allii oxalate decarboxylase; and
wherein one or more purified recombinant 6-chloronicotinic acid chlorohydrolases (cch2) is a Bradyrhizobiaceae bacterium cch2.

10) The modified bee bread of claim 6, wherein the one or more engineered cells are one or more engineered cells of claim 1.

11) The modified bee bread of claim 6, wherein the one or more purified recombinant carboxylesterases one or more purified recombinant oxalate decarboxylases, one or more purified recombinant 6-chloronicotinic acid chlorohydrolases (cch2), or one or more recombinant gene products of a 6-HNA degradation operon, or combination thereof are present in an amount effective to reduce the amount of active ingredient or neonicotinoid metabolites of one or more insecticides from a first level to a second level, wherein the second level is less than the first.

12) The modified bee bread of claim 6, wherein the one or more engineered cells are present in an amount effective to reduce the amount of active ingredient of one or more insecticides or neonicotinoid metabolites from a first level to a second level, wherein the second level is less than the first.

13) The modified bee bread of claim 6, wherein the one or more insecticides are one or more insecticides from the pyrethroid, synthetic pyrethroid, organophosphate, or neonicotinoid class of insecticides.

14) A method of providing protection from insecticides, comprising:
providing a bee bread;
providing one or more purified recombinant carboxylesterases, one or more purified recombinant oxalate decarboxylases, one or more purified recombinant 6-chloronicotinic acid chlorohydrolases (cch2), one or more recombinant gene products of a 6-HNA degradation operon, one or more engineered cells, or a combination thereof; and
introducing the one or more purified recombinant carboxylesterases, one or more purified recombinant oxalate decarboxylases, one or more purified recombinant 6-chloronicotinic acid chlorohydrolases (cch2), one or more recombinant gene products of a 6-HNA degradation operon, one or more engineered cells, or combination thereof into the bee bread.

15) The method of claim 14, wherein the bee bread is wild-type bee bread.

16) The method of claim 14, wherein the bee bread is synthetic bee bread.

17) The method of claim 14, wherein the one or more purified recombinant carboxlyesterases are Methyl Parathion Hydrolase (MPH), Pyrethroid Hydrolase (PytH), E3 carboxylesterase, E3^{Trp251Leu} carboxylesterase, Deinococcus radiodurans phosphotriesterase, or Sulpholobus acidocaldarius phosphotriesterase
wherein the one or more purified recombinant oxalate decarboxylases are a B. subtilis, Burkholderia pseudomallei, or Pantoea allii oxalate decarboxylase; and
wherein one or more purified recombinant 6-chloronicotinic acid chlorohydrolases (cch2) is a Bradyrhizobiaceae bacterium cch2.

18) The method of claim 14, wherein the one or more engineered cells are one or more engineered cells of claim 1.

19) The method of claim 14, wherein the one or more carboxylesterases one or more purified recombinant oxalate decarboxylases, one or more purified recombinant 6-chloronicotinic acid chlorohydrolases (cch2), or one or more recombinant gene products of a 6-HNA degradation operon are present in an amount effective to reduce the amount of active ingredient of one or more insecticides or neonicotinoid metabolites from a first level to a second level, wherein the second level is less than the first level.

20) The method of claim 14, wherein the one or more engineered cells are present in an amount effective to reduce the amount of active ingredient of one or more insecticides or neonicotinoid metabolites from a first level to a second level, wherein the second level is less than the first level.

21-83) (canceled)


SEQUENCE LISTING (24 sequences)

SEQ ID NO: 1 - DNA, 1035 bp, Artificial Sequence
  DQ677027.1 Stenotrophomonas sp. YC-1 methyl parathion hydrolase (mpd) gene
ctctagagattgaattcatatgcccctgaagaaccgcttgctggcccgcctgtcctgtgttgcggccgtggtggccgccacggccgccgttgcaccgttgacgctggtgtccaccgcccacgccgccgcaccgcaggtgcgcacctcggcccccggctactaccggatgctgctgggcgacttcgaaatcaccgcgctgtcggacggcacggtggcgctgccggtcgacaagcggctgaaccagccggccccgaagacgcagagcgcgctggccaagtccttccagaaagcgccgctcgaaacctcggtcaccggttacctcgtcaacaccggctccaagctggtgctggtggacaccggcgcggccggcctgttcggccccaccctgggccggctggcggccaacctcaaggccgcaggctatcagcccgagcaggtcgacgagatctacatcacccacatgcaccccgaccacgtgggcggcttgatggtgggtgagcaactggcgttcccgaacgcggtggtgcgtgcggaccagaaagaagccgatttctggctcagccagaccaacctcgacaaggccccggacgacgagagcaaaggcttcttcaaaggcgccatggcctcgctgaacccctatgtgaaggccggcaagttcaagcctttctcggggaacaccgacctggtgcccggcatcaaagcgctggccagccacggccacaccccgggccacaccacctacgtggtcgaaagccaggggcaaaagctcgccctgctcggcgacctgatactcgtcgccgcggtgcagttcgacgaccccagcgtcacgacccagctcgacagcgacagcaagtccgtcgcggtggagcgcaagaaggccttcgcggatgccgccaagggcggctacctgatcgcggcgtcccacctgtcgttccccggcatcggccacatccgcgccgaaggcaagggctaccgtttcgtgccggtgaactactcggtcgtcaaccccaagtgactcgagaatcaatcgtcgac

SEQ ID NO: 2 - DNA, 1560 bp, Artificial Sequence
  FJ688006.1 Sphingobium wenxiniae strain JZ-1 pyrethoid hydrolase (pytH) gene
tgcggccttatatcggcggcggcctgaactggacgatcatcttcaagaccgaagacggcgtgctgaaggatttccgcaccagcaataccgtcggtcctgcgatcacgggcggcatcgatgtgccgctcaacgagcggatcggtgtgttcggcgcggccagcaaggtctggaccagcaccaacgcccgcttccggctcggcacgccgaacggcttcgtgccgggtacggcgcgcgtccagctcaatcccctcatcgcgcaggtcggcctgcaatatcgcttctgatccccccggctcgaacaggaagacatgtcatgaccgtcaccgatatcatcctgatccacggcgccttgaaccgcggcgcctgctatgacgcggtcgtcccgcttctcgaagcgcgcggctaccgcgtccatgcgcccgacctgaccggccatacgcccggcgatggcggccatttgtcggtcgtcgacatggagcattatacccgcccagtcgctgacatcctggcacgggccgaggggcagtcgatccttctggggcacagcttgggcggtgcatccatctcgtggctggcgcagcaccatcccgacaaggtggccgggctgatctacctgaccgcggtcctcaccgcgcccggtataacgccggaaaccttcgtcctgcccggcgagcccaaccggggcacgccgcacgcgctggacctgatccagccggtcgacgagggacgtgggctacaggcggatttctcgcgactggaacggctccgcgaagttttcatgggcgattatcccggcgagggaatgccgcctgccgaacagttcatccagacccagtcgaccgtgccctttggcacgcccaatccgatggaggggcgcgcgctggaaatcccacgcctctatatcgaggcgctggacgatgtcgtgattccgatcgccgtgcagcgtcagatgcagaaggagttccccggtccggtcgcggtcgtgtcgctgccggccagccatgcgccctattactcgatgcccgaacggctggccgaggcgatcgccgatttcgccgatgccccggccgagtatcgccagacggcgacgaaggctgggcctgatcgaccagctggagcggacgggggtcgagccgaccgagctgatctaccgtgacgggctgtccggtgatcgcctggcggcccatgcggtgcgcaagggcggtgcctatcgcgatcggttcggcgcgccctatctcggcatccaccgtgccgatctgcagacgacgctgggccgcgcggtcggggcggagcatattcatctggcgcaccggctggagcggatcgagacggtgggcgaacgggtggcgctccacttcgtgggacgcgatcccgtcgaggccgatatcgtcgtgggtgccgacggtgttcgctccatcacccggcaatgggtgggcgggcagccgctgcgttattccgggacgagcgcgtttcgcggcatcgtgccggtcgagcggctggacgctttgcccgatccccaggcgattcagttctggatcggccaggacgcccatctgctgc

SEQ ID NO: 3 - protein, 570 aa, Artificial Sequence
  AAB67728.1 E3 [Lucilia cuprina]
MNFNVSLMEKLKWKIKCIENKFLNYRLTTNETVVAETEYGKVKGVKRLTVYDDSYYSFEGIPYAQPPVGELRFKAPQRPTPWDGVRDCCNHKDKSVQVDFITGKVCGSEDCLYLSVYTNNLNPETKRPVLVYIHGGGFIIGENHRDMYGPDYFIKKDVVLINIQYRLGALGFLSLNSEDLNVPGNAGLKDQVMALRWIKNNCANFGGNPDNITVFGESAGAASTHYMMLTEQTRGLFHRGILMSGNAICPWANTQCQHRAFTLAKLAGYKGEDNDKDVLEFLMKAKPQDLIKLEEKVLTLEERTNKVMFPFGPTVEPYQTADCVLPKHPREMVKTAWGNSIPTMMGNTSYEGLFFTSILKQMPMLVKELETCVNFVPSELADAERTAPETLEMGAKIKKAHVTGETPTADNFMDLCSHIYFWFPMHRLLQLRFNHTSGTPVYLYRFDFDSEDLINPYRIMRSGRGVKGVSHADELTYFFWNQLAKRMPKESREYKTIERMTGIWIQFATTGNPYSNEIEGMENVSWDPIKKSDEVYKCLNISDELKMIDVPEMDKIKQWESMFEKHRDLF

SEQ ID NO: 4 - protein, 337 aa, Artificial Sequence
  Deinococcus radiodurans phosphotriesterase, Genbank accession# 3GTF_A
  misc_feature (157)..(157): Xaa can be any naturally occurring amino acid
HHHHHHGDAPGGAHMTAQTVTGAVAAAQLGATLPHEHVIFGYPGYAGDVTLGPFDHAAALASCTETARALLARGIQTVVDATPNGCGRNPAFLREVSEATGLQILCATGFYYEGGGATTYFKFRASLGDAESEIYEMMRTEVTEGIAGTGIRAGVIXLASSRDAITPYEQLFFRAAARVQRETGVPIITHTQEGQQGPQQAELLTSLGADPARIMIGHMDGNTDPAYHRETLRHGVSIAFDRIGLQGMLGTPTDAERLSVLTTLLGEGYADRLLLSHDSIWHWLGRPPAIPEAALPAVKDWHPLHISDDILPDLRRRGITEEQVGQMTVGNPARLFG

SEQ ID NO: 5 - protein, 315 aa, Artificial Sequence
  Sulpholobus acidocaldarius phosphotriesterase, Genbank accession# WP_011278935
MTKIPLVGKGEISPGEMGFTLIHEHLRVFSEPVRYQWPHLYNEDEELKNAVNEVKTIMSYGVKTIVDPTVMGLGRDIRFSEKVVKETGINVIAATGLYTYTDLPFFFNGRSLEEIAELLIHDIKKGIQGTNNRAGFIKVAADEPGITRDVERAIRAAAIAQKETNVPIITHSNAHNGTGLEQQRILMEEGVDPGRVLIGHLGDTDNVDYIKKIADKGSFVGLDRYGLDLFLPIDKRNEVLLKLIKDGYLDRIMVSQDYCCTIDWGIAKPEYKPKLAPKWSMSLIFTDVIPSIKRAGVTDEQLHVIFVKNPARLFS

SEQ ID NO: 6 - DNA, 176 bp, Artificial Sequence
  Fructobacillus fructosus putative promoter
gggccctatcgccggaaacttaaaaaggtcaaggaagaaacgaattatttatagaaagtctagcactccaaaacaaagctagactttttatttaatattttattgaaagcgctttcatttataacaaaagagtgctactatatagacatagacaataggaggaaaacaaaggatcc

SEQ ID NO: 7 - DNA, 314 bp, Artificial Sequence
  Fructobacillus fructosus putative promoter
gggccctgcattaattttcttttttggttaaaataagcgaaagaacctgggcaacaaaactgtcaaaaatttcatagaaaaacgctaaaaacccttgtctgacaagggtttttctgttattatattcaagtacgccttttggggatgccgatgcaaacccgctaaaaagactggtgtatcaacgtttagcgatgatgattaaggttgcgacacgcgcggccgcgttgccatgggcgcgcaaacacaacatgtgttgtcggtttttgatcggagttagctgatttacaaaatcaacgaggaggcagaaaggatcc

SEQ ID NO: 8 - protein, 418 aa, Artificial Sequence
  Burkholderia pseudomallei Oxalate decarboxylase protein
MTNLSRRKMLAGTAGALAAAGIAVSAKAASFGNPDSPAEGAVNARNPQSLTDPGPQNPALMKEFPSFQSPPATDINGMPIFWASFNNAHKRIQNGGWAREVTQEDFAISETISGVNMRLARGGIREMHWHQQAEWAFMLDGRCRITVLDEEGRPSVQDVKTGDLWYFPPGLPHSLQGLGVDGAEFLLAFDNGRASEFNTLLVTDWIAHTPPDVLALNFGVPADAFRRIPLDNLWIFQGDDPGPLAAAQRASASSRGAPKHPFIFSMGDMKPNVKTRGGEVRIVDSTNFAVSKTIAAALVTVKPGGMRELHWHPNADEWQYYIRGDARMTVFDTGPKAQTADFRAGDVGYVKKSLGHYVQNTGTTDLVFLEIFKADRYAEVSLSDWLAHTPPQLVEAHLHIAPDVIARFPRNRPDVVPA

SEQ ID NO: 9 - DNA, 1257 bp, Artificial Sequence
  Burkholderia pseudomallei Oxalate decarboxylase nucleic acid
atgacgaatctgtcccgacgcaagatgctagccggcacggccggcgcgctcgccgccgccggcatcgccgtttccgcgaaggccgcttcgttcggcaatccggacagcccggccgagggcgcggtgaacgcgcgcaatccgcaaagcctgaccgatccggggccgcaaaatccggcgctgatgaaggagtttccttcgtttcagagtccgccggccaccgacattaacggcatgccgattttctgggcgtcgttcaataatgcgcacaagcgcattcagaacggcggctgggcgcgcgaagtcacgcaggaggatttcgcgatttccgaaacgatttccggcgtcaacatgcggctcgcgcgcggcggcattcgcgagatgcactggcaccagcaggccgaatgggcgttcatgctcgacggccgctgccggatcaccgtgctcgatgaagaggggcggccgtccgtgcaggacgtgaagacgggcgacctctggtatttcccgccggggctgccgcattcgctgcaggggctcggcgtcgacggcgccgaattcctgctcgcgttcgacaacggccgcgcgtccgaattcaacacgctgctcgtgacggactggatcgcgcacacgccgcccgacgtgctcgcgctgaacttcggcgtgcccgccgatgcattccgccgcattccgctcgacaatctgtggatcttccagggcgacgatcccgggccgctcgccgccgcgcagcgcgcgtcggcgtcgtcgcgcggcgcgccgaagcatccgttcatcttctcgatgggcgacatgaagccgaacgtgaagacgcgcggcggcgaagtgcggatcgtcgacagcacgaacttcgctgtgtcgaagacgatcgcggccgcgctcgtcacggtgaagccgggcggcatgcgcgagctgcactggcacccgaacgcggacgagtggcagtactacatccggggcgacgcgcgcatgacggtgttcgacaccggcccgaaggcgcagacggccgatttccgcgcgggcgacgtcggctacgtgaagaagagcctcggccactacgtgcagaacacgggcacgaccgatctcgtgttcctcgagatcttcaaggcggaccgctacgcggaggtttcgctgtccgactggctcgcgcacacgccgccgcagctcgtcgaggcgcatctgcatatcgcgcccgacgtgatcgcgcgctttccgcgcaaccggccggacgtcgtgccggcgtaa

SEQ ID NO: 10 - protein, 402 aa, Artificial Sequence
  Pantoea allii oxalate decarboxylase protein
MSKLTRRSFMAFAAGGTVALAAGQVHAHDSIQSFPTGGRDPGPHDPIREAENPDIVNPPSTDSGTLPNLRFSFSDAHVRKSSGGWTRQVTKRELAISTTIAGVDMRLNAGGIRELHWHKEGEWAYMTYGNARVTAFDTDGSWFIDDIGPGDLWYFPPGIPHSIQGIGPDGCEFLLAFDDGGFDEDSTFLLSDWFKHIPPDILAKNFNVPVSTFDHLPSPADEYIFAAKVPGSLESVAVKGATKAKRTFTHHMLAQEPIKAPGGTVRITDSSTFKVSKTIAAALVEIAPGGLRELHWHPNNDEWQYYLEGEGRMGVFASSGQARTFDYRAGDVGYVPFAMGHYVENTGKGPLRFLELFKSDYYADISLNQWLAGTPRELVKAHLQLDDAFMDALSLQKNPVVK

SEQ ID NO: 11 - DNA, 1209 bp, Artificial Sequence
  Pantoea allii oxalate decarboxylase nucleic acid
atgagtaaattaacccgaagaagttttatggcgtttgccgcaggcgggacggtggcgctggcggctggacaggttcatgcgcatgacagcattcagtcctttccgaccggtggtcgcgatcccgggccgcacgatcccatccgcgaagcagaaaatccggacattgttaatccaccttcgaccgacagcgggacgttaccgaacctgcgtttttcattcagtgatgcgcacgtgcgcaaaagcagcggcggatggacgcgccaggtcaccaagcgtgaattagccatttcgaccaccattgccggtgtggacatgcgcctgaatgccggcggcatccgcgaattacactggcataaagagggcgaatgggcctacatgacctatggcaacgcccgcgtcaccgcgtttgataccgatggcagttggtttatcgatgatattggcccgggagacttatggtatttcccgccgggcatcccgcactcaattcagggcatcgggcctgatggatgcgaatttctgctggccttcgacgatggcggatttgatgaggacagcacgtttctgttatccgactggttcaaacatatcccgccggacattctggcgaaaaacttcaacgtcccggtatccaccttcgaccacctgccttcgccagcggatgaatatatttttgccgcgaaggtgcccggcagcctggagtcagtggcggtgaaaggcgcaaccaaagccaaacgcacctttacccaccacatgctggcacaggaacccataaaggcaccgggcggcacggtcagaatcaccgactcctcaacctttaaggtgtccaaaactatcgcggcagccctggttgaaatcgcccccggcggattacgcgagttgcactggcacccgaataacgatgaatggcaatactacctggaaggggaaggacgcatgggcgtttttgcctcatcgggtcaggcaagaacatttgattatcgggcaggggatgtcggctatgtgcccttcgccatgggccactatgttgaaaacaccggcaaggggccgctgcgtttcctggagttattcaaaagtgactattacgccgatatctcactcaatcagtggctggccggtacgccgcgtgagctggtaaaagcgcatctccagttagatgacgcatttatggatgcgctgtcgctgcagaaaaatcctgtggtgaaataa

SEQ ID NO: 12 - DNA, 2688 bp, Artificial Sequence
  Cassette expressing transporter and cch2 (6-CNA Chlorohydrolase; including HindIII and BamHI cloning ends) (nucleic acid)
aagcttaggaggtctagaaatatgatgcgacgtgtacttgtccttggtatctttatgattactcaatctgtagttgcagaagctgcagatgaaatcaaaattggtgaaatcaactcttattctacagcagctagttttactgaaccatatcgtaaaggttggcaacttgcagtagatgaaatcaatgctaaaggaggtgttcttggcaagaaacttatcgttattgcaaaggacgatgctggaaaacctgctgatgcgattactgctgcgaatgatcttatggcaaacgaaggtgtcgttatgttagctggtacatttctttctcatgtagctctcgcagtatcagatttcgccaaacagaaaaagatcgtgtttatggctggtgaacctttgacagatgcacttacgtggtcacaaggtaataagtacacattccgtcttcgcccaaatacctatatgcaagctgcaatgttggctgaagaggcagcgaaactgccagcaaaacgttgggctaccattgctccgaactatgagtttggacaatctgcagttgcagctttcaagaaacttttgtctcaaaaacgtccagatgttgaatgggtaggagaacaatggccacctctcggcaagattgatgctggaccagtagttcaagcacttgctgcaactaaaccagatgcaattatgaatgcagtatttggagttgatctcgccaaatttgtacgtgaaggtaatgcacgtgctctgttcaaagatagatcagttgtaagtttcttgacaggtgaaccagagtttctcgatcctctgaaagacgattcacctgttggctggattgtgacaggttatccttggtatggaatcaaaactccagaacatgatgcgtttctcaaagcatatcaagcaaaacataaagattatcctcgtcttggttctgtagttggctatcaacccattatggctgcagccgctatgcttaacaaagcaggatcaactgatactgataaacttatcgcagcgggtgaagatttgaagatggaatcaccatttggaccaatcgtactccgtaaaattgatcatcaagctacattaggagattatatcggtaaaatcgctattaaggatggtatgggagtgatggtagacttcaaatactttgatggagctcaatatctacctacagatgcagaaatcggaaaaatgcgtccaaaggattaaatacgttcactaaggagggtctctagaatgagtcttattgcaatcaaaaatgcagattggattcttactatggatggagctcgtcgagttattgcagatggatctatcttgattgacaaagatcgtattcgagcaattggtaaatctgctgtgattgaaatccctccagaaacaactaaggttattgatggtcgtggaaaagttgtattaccaggattgattgattctcatacacaccattcacttcatttaggtcgtggacttgcagatgaatgtgacattcaaacgtttctgtatcgacgtttgtatcccattgaagcatcacttaatgatgaagatgcttacattagtgcattgttatgtcaacttgaaatgattaagtctggtactacatcgattatcgatgctggaaactactttcctgaagcaacgcttcgtgcatttggaacaactggtatgcgtggagttgtagcacgaagtacgtttgatattccaacatcttcacttggatcattgccagcacaagtgtttgctgaagagaccaatgttgcattgaaacgtgctgaagagtttgtagaacgaaacagtggtgcttgtgatggtcgtgtgcaagcatggttacaattgcgtgttcttccgaattgttctgatgaactttgccgtggcttaaaaagtattgctgatcgacttggagttcgttatgaagcacatgcagccttcactaaagaagtctatgaagcctctaaactgcagtttggtaaatctgaagttcgtcgcttggatgacctcggtatcctcggtgaaggattattgctcgcgcatatgggatggttaacgccacgtgacatcttgttactcattagttctaaaactaatgttgttctttgtccaacatctagtgtacatcaagcgatgggtagcattgcatttggacatgtccctgagttgttagaaatgggagttaacgttgctcttggaacggatggcggtcctcatggaactaatgatcttgttcgccaaatctttgtagctgcaggaggctataaggaagttcgtcttgatgcaacaatcatgccacctgagacagtacttgaaatggcaaccgttaacggtgctcgtgcaatgggaatgtcagatcaagtaggatctattgaacctggtaagaaagcagacattacaatctttgattctcgtcgacctgaatggcgtccattgcacaatccagtcgcgaatctcgtatattgcgcaaacggaaattctgctgataccgttattgtagatggacgtatcttgatggagaatcgtaaaattctcaccttcaacgaagatgacgtaattactgaagctcaacgtcgatcagttgagattggtgctcgtgcaggattgttagaatatggacgaccacgctggccagtccattgaagagatataaaaagccagattattaatccggcttttttattatttggatcc

SEQ ID NO: 13 - protein, 841 aa, Artificial Sequence
  Cassette expressing transporter and cch2 (6-CNA Chlorohydrolase) (protein)
MMRRVLVLGIFMITQSVVAEAADEIKIGEINSYSTAASFTEPYRKGWQLAVDEINAKGGVLGKKLIVIAKDDAGKPADAITAANDLMANEGVVMLAGTFLSHVALAVSDFAKQKKIVFMAGEPLTDALTWSQGNKYTFRLRPNTYMQAAMLAEEAAKLPAKRWATIAPNYEFGQSAVAAFKKLLSQKRPDVEWVGEQWPPLGKIDAGPVVQALAATKPDAIMNAVFGVDLAKFVREGNARALFKDRSVVSFLTGEPEFLDPLKDDSPVGWIVTGYPWYGIKTPEHDAFLKAYQAKHKDYPRLGSVVGYQPIMAAAAMLNKAGSTDTDKLIAAGEDLKMESPFGPIVLRKIDHQATLGDYIGKIAIKDGMGVMVDFKYFDGAQYLPTDAEIGKMRPKDMSLIAIKNADWILTMDGARRVIADGSILIDKDRIRAIGKSAVIEIPPETTKVIDGRGKVVLPGLIDSHTHHSLHLGRGLADECDIQTFLYRRLYPIEASLNDEDAYISALLCQLEMIKSGTTSIIDAGNYFPEATLRAFGTTGMRGVVARSTFDIPTSSLGSLPAQVFAEETNVALKRAEEFVERNSGACDGRVQAWLQLRVLPNCSDELCRGLKSIADRLGVRYEAHAAFTKEVYEASKLQFGKSEVRRLDDLGILGEGKTNVVLCPTSSVHQAMGSIAFGHVPELLEMGVNVALGTDGGPHGTNDLVRQIFVAAGGYKEVRLDATIMPPETVLEMATVNGARAMGMSDQVGSIEPGKKADITIFDSRRPEWRPLHNPVANLVYCANGNSADTVIVDGRILMENRKILTFNEDDVITEAQRRSVEIGARAGLLEYGRPRWPVH

SEQ ID NO: 14 - protein, 430 aa, Artificial Sequence
  porin-like protein, nicP (NCBI accession NP_746069.1)
MQGFTAPRFSSSARLAGGCIAAALCAEATAEEAGFIEGARATLQARNYFFSRDYADIKGASTQSRTQEWAQGFILNASSGYTQGTLGVGVDVTGLLGFKLDSSPEHARSGLLPSLDSGKSADEYSRLGAAIKFKVSQTELKVGELMPNLPVLLFSDLRLLPPTYQGAMLESREFAGLTLSAGQFRSTSLRDSSNSQKMYALVNDPINPARLARFTSDRFNYVGADYAFNDNRTSVGVWQAQLEDIYQQRFYSFKHAEPLGSWTLGVNAGYFDAREDGSKVAGDYDNHALFSLFSAKTGGHTFYVGYQQIGGDDGFIQVGANTNPMGNTLPTYEFSAPGERSWQVRHDYNFVALGLPGLTSTLRYVKGRDVETGLGFEGRDRERDLDLAYVVQSGPLAGLGIRVRNVMARSNYRTDIDENRLILSYTIKVF

SEQ ID NO: 15 - protein, 437 aa, Artificial Sequence
  metabolite transport protein, NicT (NCBI accession NP_746070.1)
MPIANATTVHSDIDHGTKALYSKITWRLIPFLCFCYLAAYLDRINVGFAKLQMLEDLQFSTAAYGLGAGLFFVGYIIFEVPSNLILQRVGAKLWIARIMITWGLLSACTMFVTSTTQFYILRFLLGAAEAGFLPGVLYYLTMWYPTYRRGRIIALFMIGLPLSSVIGGPISGWIMGHFDQVQGLHGWQWLFLLEAIPSVLLGILTFWALPNHFQQAKWLSADDKAQLAADLAADDAEGKDSKHSFRDGFFNLKVWMLGGIDFSILLSAYAMGFWMPTFIRDAGVSDTFHIGLLTAIPSLAALAGMLMIGASSDRHRERRWHIIVPFIIGAIAMASSTLFSQNLVMTVVLFAIASAAIIGAVPVFFSLPATFLKGTAAATGFALACSVANIAGLVSNSLMGVVTDLTGTSHAALWVFAGCLILSCFLVIALPAKLVNR

SEQ ID NO: 16 - protein, 213 aa, Artificial Sequence
  maleamate amidohydrolase, NicF (NCBI accession NP_746071.1)
MSDAQSARDNYQGVWGQRIGFGRKPALLMIDFMQGYTTPGAPLYAPGVVAAVEQAAGLLALARDCGTLVVHTNIRYQPPHFADGGVWVRKAPVMKDMVEGNPLAAFCEAVAPQAGEVVLSKQYASAFFATSLAPLLHAQGVDTVVLAGCSTSGCIRASAVDAMQHGFRTIVVRECVGDRHSDPHEANLFDIDSKYGDVVTRQDAMQQLRHLAG

SEQ ID NO: 17 - protein, 250 aa, Artificial Sequence
  maleate isomerase, NicE (NCBI accession NP_746072.1)
MTQLYRIGQIVPSSNTTMETEIPAMLNARQAIRPERFTFHSSRMRMKQVKKEELAAMDAESDRCAVELSDAKVDVLGYACLVAIMAMGLGYHRQSEKRLQQATADNDALAPVITSAGALVEALHVMKAKRIAIVAPYMKPLTELVVNYIREEGFEVQDWRALEIPDNLAVARHDPANLPGIVAGMDLEGVDVVVLSACVQMQSLPAVAKVEAQTGKPVVTAAIATTYAMLKALDLEPIVPGAGALLSGAY

SEQ ID NO: 18 - protein, 268 aa, Artificial Sequence
  N-formylmaleamate deformylase, NicD (NCBI accession NP_746073.1)
MSTFVAGGNVSANGIRQHYLRYGGKGHALILVPGITSPAITWGFVAERLGHYFDTYVLDVRGRGLSSSGPDLDYGTDACAADIPAFAAALGLDSYHLLGHSMGARFAIRAAAQGAPGLQRLVLVDPPVSGPGRRAYPSKLPWYVDSIRQATVGMSGDDMRAFCATWSDEQLALRAEWLHTCYEPAIVRAFDDFHEVDIHQYLPAVRQPALLMVAGRGGVIEPRDIAEMRELKPDIQVAYVDNAGHMIPWDDLDGFFAAFGDFLDHPLV

SEQ ID NO: 19 - protein, 382 aa, Artificial Sequence
  6-hydroxynicotinate 3-monooxygenase, NicC (NCBI accession NP_746074.1)
MRGRQKIAIVGAGLGGAAAATLLQQAGFDVEVFEQAPAFTRLGAGIHIGPNVMKIFRRMGLEQKLELMGSHPDFWFSRDGNTGDYLSRIPLGEFARREYGAAYITIHRGDLHALQIEAIQPGTVHFGKRLEKIVDEGDQVRLDFADGTHTVADIVIGADGIHSKIREELLGAEAPIYSGWVAHRALIRGVNLAQHADVFEPCVKWWSEDRHMMVYYTTGKRDEYYFVTGVPHEAWDFQGAFVDSSQEEMRAAFEGYHPTVQKLIDATESITKWPLRNRNPLPLWSRGRLVLLGDACHPMKPHMAQGACMAIEDAAMLTRCLQETGLSDHRTAFALYEANRKERASQVQSVSNANTWLYSQEDPAWVYGYDLYGQQLESGEAA

SEQ ID NO: 20 - protein, 350 aa, Artificial Sequence
  2,5-dihydroxypyridine 5,6-dioxygenase, NicX (NCBI accession NP_746075.1)
MPVSNAQLTQMFEHVLKLSRVDETQSVAVLKSHYSDPRTVNAAMEAAQRLKAKVYAVELPAFNHPTAMGNDMTAYCGDTALTGNLAAQRALEAADLVVDTMMLLHSPEQEQILKTGTRILLAVEPPEVLARMLPTEDDKRRVLAAETLLKQARSLHVRSKAGSDFHAPLGQYPAVTEYGYADEPGRWDHWPSGFLFTWPNEDSAEGTLVLDVGDIILPFKNYCRERITLEIEKGFITGIHGGFEAEYLRDYMKYFNDPEVYGISHIGWGLQPRAQWTAMGLHDRNDGMCMDARAFYGNFLFSTGPNTEVGGKRKTPCHLDIPLRNCDIYLDDKAVVLAGDVVAPEESRAR

SEQ ID NO: 21 - protein, 158 aa, Artificial Sequence
  HTH-type transcriptional repressor, NicR (NCBI accession NP_746076.1)
MSKKTTPSSAPLDNTPYDVTEQVGHLLRKAYQRHTAIFQQQACDPQLTSIQFVTLCALRDHGPSSQAELIKATAVDQATIRGIVERLKARELVQLSPDPGDRRKVIVELTESGAALLDAMIPCARQISELSMGSLNAGERVAILYLLRKMIDSDENAG

SEQ ID NO: 22 - protein, 157 aa, Artificial Sequence
  nicotinate dehydrogenase subunit A, NicA (NCBI accession NP_746077.1)
MQTTISLQVNGQPVEVSAMPDTPLLLILRNDLCLNGPKYGCGLGECGACTVIIDGVAARSCVIPLAGAAGRNITTLEGLGSKAAPHPVQQAFIDEQAAQCGYCMNGMIMTAKALLDRIPEPSDEQIRNELSANLCRCGTHVEILRAVRRAAETRRKP

SEQ ID NO: 23 - protein, 1187 aa, Artificial Sequence
  nicotinate dehydrogenase subunit B, NicB (NCBI accession NP_746078.1)
MNHSQQVPSRDQLLAKTGVLLIVDQITPPSGPVAKGVTPTVKERELALFIAVSDDGMVYAFNGHVDLGTGIRTSLAQIVAEELDLRMDQVHMVLGDTERAPNQGATIASATLQISAVPLRKAAATARRYLLQQAALRLGCPPEMLRIEDGTVIASNGSTLSFAELVQGKNHQLHIADDAPLKAIEDYRLVGRSAPRVDIPGKATGELTYVHDMRLPNMLHGRVIRPPYAGHDSGDFVGNSLLAVDESSIAHLPGVVAVVVIRDFVGVVAEREEQAIRAAHELKVSWKPFTGKLPDLSDVAQAIRDNPRVQRTVLDQGDVDGGIANASQRLSRSYLWPYQLHASIGPSCALADFTAGQIRVWSGTQNPHLLRADLAWLLACDEARIEIIRMEAAGCYGRNCADDVCADAVLLSRAVQRPVRVQLTREQEHVWEPKGTAQLMEIDGGLNADGSVAAYDFQTSYPSNGAPTLALLLTGAVEPVPALFEMGDRTSIPPYDYEHMRVTINDMTPLVRASWMRGVSAMPNSFAHESYIDELAFAAGVDPVEYRLKHLSDPRAIDLVKATAERAQWQPHTRPMQTQAEGDVLRGRGFAYARYIHSKFPGFGAAWAAWVADVAVDRRTGEVAVTRVVIGHDAGMMVNPEGVRHQIHGNVIQSTSRVLKEQVSFEESTVASKEWGGYPILTFPELPAIDVMMLPRQHEPPMGSGESASVPSAAAIANAIFDATGIRFRELPITAERVRAALGGEGQGPDAPAPAQPSTKRSKWWFGSLAGVFGAALGMLATALPWRAEIAPVTPPGVGSWSAAMLERGRQVAAAGDCAVCHTVSGGKANAGGLAMDTPFGTLYSTNITPDPETGIGRWSFAAFERAMREGISRDGRHLYPAFPYTSFRNINDADMQALYAYLMSQTPVRQEAPANQMRFPFNQRPLMAGWNARFLQRGEYQPDPQRSAQWNRGAYLVDGLGHCTACHSPRNLMGAEKGGSSYLAGGMVDGWEAPALNALGKSSTPWSEDELFNYLSTGFSEKHGVAAGPMGPVVSELATLPKSDVRAIAHYLSSLEGEPQALAANAAPQVDTHVSLSNGERVFKGACLGCHSDGLGPKLFGVSPSMAVNSNVHSDLPDNLLRVVLHGIPTPATRDLGYMPGFKDSLSDRQVADLAAYLRHRFAADKPAWQGLASKAAQVRANPGSH

SEQ ID NO: 24 - protein, 212 aa, Artificial Sequence
  HTH-type transcriptional repressor, NicS (NCBI accession NP_NP_746079.1)
MQTRKTGVRAQQADRTRDNILKAAVKVFSKEGFTGGRIEQISTLAKSNDRMIYYYFGSKEKLFISVLEHIYASFNQAEAKLRLDLADPEQALRELVAFIWDYYVRHPEFVTILATENLHQGLHARKSQNLRALSGEAVGVLRPIIEAGQAKGLFRDDICITHAYLMIASLCYFYNSNRHTLSSFLAVDLADKQAKADWLTFISDLALRGLRR